(19) United States
(12) Patent Application Publication
(10) Pub. No.: US 2026/0199252 A1
(43) Pub. Date: Jul. 16, 2026

(54) NUCLEIC ACID-LIPID PARTICLES

(71) Applicants: UNIVERSITEIT UTRECHT HOLDING B. V., Utrecht (NL); STICHTING AMSTERDAM UMC, Amsterdam (NL)

(72) Inventors: Lucas CZENTNER COLOMO, Utrecht (NL); Johanna Martina Maria DEN HAAN, Amsterdam (NL); Maarten Klaas NIJEN TWILHAAR, Amsterdam (NL); Gerrit STORM, Utrecht (NL); Cornelus Franciscus VAN NOSTRUM, Utrecht (NL)

(73) Assignees: UNIVERSITEIT UTRECHT HOLDING B. V., Utrecht (NL); STICHTING AMSTERDAM UMC, Amsterdam (NL)

(21) Appl. No.: 19/137,327

(22) PCT Filed: Dec. 20, 2023
(86) PCT No.: PCT/EP2023/086994

§ 371 Date: Jun. 10, 2025

(30) Foreign Application Priority Data
Dec. 20, 2022 (EP) 22214821.5

(51) Int. Cl. A61K 9/48; A61K 9/00; A61K 31/7105; A61K 31/711; A61K 39/00; A61K 39/39; A61K 45/06; A61P 35/00

(52) U.S. Cl. CPC A61K 9/4858; A61K 9/0019; A61K 9/4833; A61K 31/7105; A61K 31/711; A61K 39/0011; A61K 39/39; A61K 45/06; A61P 35/00; A61K 2039/53; A61K 2039/54; A61K 2039/55555

(57) ABSTRACT

The present invention pertains to a composition comprising nucleic acid-lipid particles, wherein the nucleic acid-lipid particles are characterized by encapsulation of nucleic acids in the lipid bilayer. The present invention furthermore pertains to said composition, for use in preventing and/or treating a disease, in particular for use in preventing and/or treating cancer. The present invention furthermore pertains to a method for producing a composition comprising said nucleic acid-lipids particles.

### TECHNICAL FIELD

The present invention relates to nucleic acid-lipid particles, particularly for use in therapy such as in treating or preventing cancer, infectious disease or autoimmune disease.

### BACKGROUND OF THE INVENTION

Nucleic acid adjuvants have gathered attention because they can induce potent cellular immune responses against antigens, which is critical for example in cancer therapy. However, their physicochemical properties, pharmacokinetics and toxicology profiles have limited their application in cancer immunotherapy. This is in particular because nucleic acids are hydrophilic highly negatively charged molecules with poor cell entry capability. When administered intravenously, they have a short half-life and are rapidly degraded by blood nucleases. These unfavourable aspects often force the use of high doses that can induce non-specific and even toxic effects, especially under systemic administration.

Current delivery of nucleic acids is based on using mostly cationic lipids, cationic polymers, or multivalent cations. These cationic molecules interact with the negatively charged phosphate groups of the nucleic acids, allowing their encapsulation in forming nano or microparticles. Cationic liposomes and cationic polymers have been widely used for in vitro transfection of cells. They are based on molecules that are positively charged at physiological pH (7.4). They successfully protect the nucleic acids from nuclease degradation and enhance cellular uptake.

However, some of these delivery systems are highly toxic in vivo and not physically stable in plasma, limiting their application for systemic administration of nucleic acids. Recently, novel lipid nanoparticles (LNPs) have been developed for the systemic administration of nucleic acids (U.S. Pat. No. 8,058,069B2). They are based on ionisable cationic lipids, which are positively charged at acidic pH (<6.5) but not at physiological pH (7.4). Including the ionisable cationic lipids allows efficient encapsulation and intracellular release of the nucleic acid. They are being successfully used in humans for siRNA delivery and in mRNA COVID-19 vaccines. Although LNPs are serum stable, the ionisable cationic lipid employed in the LNPs technology negatively impacts RNA stability (Packer et al, Nat Commun Vol 12, 2021, article number 6777) and can induce dose-limiting inflammatory responses as shown in preclinical and clinical studies (Ndeupen et al, iScience. 2021 Dec. 17;24 (12): 10347; Tahtinen et al, Nat Immunol Vol 23, 2022, pages 532-542). As an example, cationic lipids (as well as cationic polymers and multivalent cations) activate the intracellular inflammasomes, e.g NLRP3 promotes IL-1β expression and induces cell death. Such inflammatory responses can limit a broader application of the technology, especially outside of the vaccination field, when often systemic applications are required and nucleic acids are used for gene therapy and to produce therapeutic proteins. Additionally, the inflammatory profile of LNPs is not desired for nucleic acid adjuvants, which are inherently immunostimulatory and have already a limited therapeutic window due to the induction of non-specific effects which can be toxic. Furthermore, activation of inflammasomes and IL-1β mediated inflammation is not desired for cancer therapy, as it has been found to suppress anti-tumor immunity leading to the progression of human melanoma, gastric and breast cancer (Yu et al., Sig Transduct Target Ther Volume 6, 2021, article number 128).

Multivalent cations, like calcium and magnesium, have been used to complex nucleic acids with neutral and anionic liposomes (Kapoor et al., International Journal of Pharmaceutics, Volume 432, Issues 1-2, 2012, Pages 80-90). However, these multivalent cations can cause the liposomes to aggregate, which make them harder to produce, affects particle stability and their biological properties. Additionally, they can also make the nucleic acids less stable and less active (Dallas, A et al., Artificial nucleases, 2004, Pages 61-88). This method has been used mainly in vitro studies without clinical application. Furthermore, Michanek et al (Biochimica et Biophysica Acta 1798, 2010, Pages 829-838) have explored how tRNA associates with liposomes in the absence of divalent cations, when added to preformed nanoparticles, but found that the tRNA is merely adsorbed to the external surface of the liposomes to a low degree and in a weak manner. Accordingly, the lipid composition that are used are not suitable for pharmaceutical drug carrier applications.

It is an objective of the present invention to overcome one or more of the above-mentioned or other problems.

### SUMMARY OF THE INVENTION

The present inventors developed a method to produce stable anionic liposomes encapsulating nucleic acids, particularly in their lipid bilayer(s). Encapsulation of the nucleic acid by the respective nucleic-acid lipid particles, allows for:
higher stability of the nucleic acids;
stronger association of the nucleic acids with the particles which is important for realizing the therapeutic functionality e.g. all as compared to nucleic acids adsorbed to the particles (such as in Michanek et al (Biochimica et Biophysica Acta 1798, 2010, Pages 829-838); and
higher resistance of the nucleic acids to the action of nucleases when the particles are released in a patient's bloodstream; and
no substantial localization of RNA on the surface of the nanoparticles to avoid increasing permeability of vessels and potential immunotoxicity reactions (Fischer, et al. Blood, The Journal of the American Society of Hematology 110.7, 2007, Pages 2457-2465).

The liposomes obtainable by the method of the invention make them particularly attractive for the targeted delivery of nucleic acid adjuvants to the immune system. In particular, it was found that the liposomes encapsulating nucleic acids improves immune stimulation (e.g. stimulation by activation of toll-like-receptors) after enhanced adjuvant uptake by immune cells (e.g. dendritic cells, macrophages, lymphocytes). The inventors have shown that encapsulation in anionic liposomes protects nucleic acids and makes them better accessible to target immune cells to boosts their biological effects. The anionic liposomes containing nucleic acids in the bilayer(s) are bio-compatible and physically stable. Incorporating the nucleic acid(s) in the bilayer(s) better conserves nucleic acid content and biological activity during storage (shelf life of at least 5 months instead of weeks), in comparison to not having the nucleic acid(s) in the bilayer(s) as in the art. Since the method of the invention requires minimal or no cationic lipid, the problems associated with cationic liposomes (e.g. toxicity and low physical stability in vivo) can be overcome. In contrast to cationic nanoparticles, anionic liposomes do not activate inflammasomes (Li, T. et al., Nanomedicine Volume 14, 2018, Pages 279-288) and are naturally immunotolerant (Benne et al., Journal of Controlled Release, Volume 291, 2018, Pages 135-146).

The current invention pertains to a method for producing stable liposomes with a highly efficient encapsulation of nucleic acids in the lipid bilayer(s). Microfluidics technology was used to demonstrate encapsulation of poly (I:C), a model RNA-based nucleic acid adjuvant, within the bilayers of liposomes. As exciting observation, these RNA-liposomal formulations appear to require little or no cationic molecules to encapsulate RNA. This is an advantage, since the inclusion of cationic molecules increases the immunogenicity and toxicity of the formulation (Patil et al., AAPS J 6, 13-22 (2004)). Encapsulation of nucleic acid in the lipid bilayer(s) is preferably achieved by using an appropriate organic solvent with minimal or no cationic molecules. This organic solvent is preferably an ethanol solution with at least 80% ethanol. To achieve efficient encapsulation of nucleic acids in the lipid bilayer and in stable particles, the organic solvent is mixed with an aqueous solution comprising one or more nucleic acid forms preferably by providing a laminar flow, e.g. using a relatively low flow rate (e.g. 25-5000 μL/min). Preferably, the initial mixing of organic and aqueous solutions is followed by the mixing of one or more additional aqueous solution (e.g. RNase free aqueous medium) or organic solutions, e.g. using a relatively low flow rate (e.g. 10-20000 or 25-5000, preferably 25-500 L/min). This may increase formulation reproducibility and allows for better control of particle size.

Without being bound by theory, hydrophobic interactions may be the mechanism for the encapsulation of RNA by the liposomes, particularly in the lipid bilayer(s). Through mixing with one or more organic solutions, the nucleic acid molecules change their conformation, exposing hydrophobic moieties that can interact with lipid tails, in particular under laminar mixing.

The discovery of the present inventors is surprising, since encapsulation of nucleic acids in anionic or neutral liposomes is generally considered to be very inefficient and requires a previous condensation of the nucleic acids with cationic molecules (Bailey et al. Biochim Biophys Acta. 2000 Sep. 29; 1468 (1-2): 239-52, Patil et al., AAPS J 6, 13-22, 2004, Kapoor et al., International Journal of Pharmaceutics, Volume 432, Issues 1-2, 2012, Pages 80-90). However, the use of multivalent cationic ions such as Ca²⁺ to encapsulate nucleic acids in anionic liposomes lead to the formation of aggregates and significantly increase of the mean size of the liposomes to values significantly higher than about 220 nm (Kapoor et al. and Patil et al.). This is a disadvantage if the liposomes containing nucleic acids are administered as an injection, as it is then required to deploy a sterile filtration step in the manufacturing process. Sterile filtration is done by passing the formulation through a 0.22 μm rated sterilizing-grade filters. Any particles present that are greater than 220 nm can cause significant reduction in the filter throughput or eventually clog the filters, negatively affecting the manufacturability of the formulation. The current invention allows the efficient encapsulation of nucleic acids in nucleic acid-lipid particles, e.g. anionic liposomes, that have a mean size below 220 nm, making it suitable for a sterile filtration step. Additionally, in contrast to prior art thin film method, the present microfluidics method used in the invention allows the upscaling of the production. The present inventors also found that a surprisingly lower dosage of nucleic acid is required, in comparison to prior art methods, in order to achieve therapeutic effect.

Structural characterization of the liposomes obtained by the methods described herein show that the RNA-containing nanoparticles had the typical liposomal vesicle structure and, strikingly, the RNA cargo appeared to be mainly embedded within the hydrophobic region of the liposomal bilayers. The liposomes of the invention typically have an aqueous core and one or more lipid bilayers containing RNA. The organization of the lipids in bilayers (lamellar phase) is energetically the most favourable, providing a very stable structure. In comparison, nucleic acid-LNPs in the prior art have a solid core containing RNA, cationic lipids and cholesterol with low water content (27% water volume vs total core volume) and lipids organized in inverted hexagonal phases (H_{∥}) as compared to regular liposome particles (Arteta et al 2018, PNAS Vol. 115| No. 15). Complexation of nucleic acids and anionic liposomes using cationic ions (e.g., Ca²⁺) can reorganize the lipids from lamellar phase to a less physical stable inverted hexagonal phase (H_{∥}) (Barran-Berdon et al., Langmuir, 2014, vol. 30, no 39, pages 11704-11713). The RNA-containing liposomes of the present invention were physically stable during storage for 5 months at 5° C., and −20° C. The localization of the nucleic acids in the lipid bilayers devoid of water may explained the preservation of nucleic acid content and biological activity during storage. Experiments showed that anionic liposomes containing RNA remain intact, do not aggregate nor release their content, and protected the RNA from nuclease degradation when incubated with human plasma at 37° C. Immunostimulant activity was studied ex vivo using mouse splenocytes and in vivo in healthy and tumor-bearing mice. The poly (I:C) encapsulation enhances the activation of dendritic cells, T-cells and NK cells and the production of anti-viral cytokines without noticeable toxicity.

The liposomes obtainable by the method were stable in plasma, stable upon storage and can efficiently be taken up by immune cells. The liposomes with encapsulated nucleic acids, e.g. in the bilayer(s), are shown to have desirable physical characteristics for (clinical) application such as cancer therapy, including desirable size distribution, polydispersity, and charge. Furthermore, the inventors found that fine-tuning of the lipid composition of the liposomes can direct their immunological activity to achieve either predominantly immunostimulatory or immunosilent action. It was shown that incorporating poly (I:C) in anionic liposomes augments the vaccine effectiveness of liposomes containing a model ovalbumin peptide. It was shown that intravenous administration of encapsulated poly (I:C) in the liposomes according to the invention as a monotherapy or in combination with an immune checkpoint inhibitor antibody (ICI) in multiple mouse tumor models, but not with ICI as monotherapy or in combination with free poly (I:C), delayed tumor growth and improved overall mice survival. Overall, the characteristics of the particles/liposomes of the invention make them attractive for the targeted delivery (e.g. of nucleic acids adjuvants) to the immune system and/or specifically targeted delivery to the spleen of a subject. It is found that the addition of an anionic lipid in the particle improves the uptake by spleen dendritic cells. In one experiment, the present inventors assessed the uptake and activation by spleen conventional type 1 dendritic cells (cDC1s) ex vivo. This is a cell population that expresses TLR3 and, in the presence of poly (I:C), upregulates maturation markers. In this experiment, the inventors wanted to understand the impact of EPG concentration on particle uptake and maturation by cDC1. The inventors selected five formulations containing an increasing concentration of EPG (0, 4, 17, 33, 67% molar ratio), decreasing concentration of EPC and constant concentration of cholesterol (33% molar ratio). It was shown that an increasing molar ratio of EPG enhances nanoparticle uptake by cDC1s cells.

In an aspect, the present invention relates to a method for preparing a composition comprising nucleic acid-lipids particles, e.g. comprising at least 2, 10, 100, 1000, 10000, 100000 or more particles, the method comprising:
a) providing one or more organic solution comprising at least one anionic lipid and/or at least one neutral lipid;
b) providing one or more aqueous solution comprising one or more (type of) nucleic acid;
c) combining, preferably by laminar flow mixing, the one or more organic solution and the one or more aqueous solution, thereby producing the composition comprising nucleic acid-lipids particles.

In an aspect, the present invention relates to a composition comprising anionic nucleic acid-lipid particles, wherein the anionic nucleic acid-lipid particles comprise:
one or more (type of) nucleic acid;
one or more (anionic) lipid bilayer comprising (and/or formed by) at least one anionic lipid and/or at least one neutral lipid.

Preferably, at least part of the one or more nucleic acid is encapsulated by the anionic nucleic acid-lipid particles and/or less than 10 wt. % of the one or more nucleic acid is exposed on the outside of the anionic nucleic acid-lipid particles and/or less than 10, 5, or 1 wt. % of the one or more nucleic acid is covalently bound to a lipid comprised by the anionic nucleic acid-lipid particles.

Preferably, the nucleic acid-lipid particles do not comprise cationic lipid, cationic polymer or multivalent cationic ion (e.g. Ca2+) or comprise at most 20 mol % of cationic molecule, such as cationic lipid, cationic polymer or multivalent cationic ion (e.g. Ca2+), relative to total mol lipid of the nucleic acid-lipid particles. Cationic molecules/ions may lead to undesired aggregation of the particles and/or undesired degradation of the one or more nucleic acid.

In an aspect, the present invention relates to the composition according to the invention, for use in preventing and/or treating a disease.

### DETAILED DESCRIPTION OF THE INVENTION

### Composition of the Invention

The present invention relates to a composition comprising nucleic acid-lipid particles e.g. comprising at least 2, 10, 100, 1000, 104, 105, 106, 107, 108 or more particles, wherein the nucleic acid-lipid particles comprise:
one or more nucleic acid;
one or more lipid bilayer comprising (and/or formed by) at least one anionic lipid and/or at least one neutral lipid;

wherein the nucleic acid-lipid particles are anionic.

Preferably at least part of the one or more nucleic acid is encapsulated by the nucleic acid-lipid particles, more preferably at least 1, 2, 3, 4, 5, 6, 7, 8, 9, 10, 15, 20 wt. % of the one or more nucleic acid is encapsulated by (or localized within) the nucleic acid-lipid particles, such as within the outer boundary of the nucleic acid-lipid particles.

In addition or alternatively, at least part or at least 1, 2, 3, 4, 5, 6, 7, 8, 9, 10, 15, 20 wt. % or all of the one or more nucleic acid is comprised in the hydrophobic region of the one or more lipid bilayer, preferably in the form of nucleic acid-lipid complexes (e.g, wherein hydrophobic moieties of the one or more nucleic acid interact with lipid tails of the one or more lipid bilayer. Preferably, at least part or at least 1, 2, 3, 4, 5, 6, 7, 8, 9, 10, 15, 20 wt. % or all of the one or more nucleic acid is not in contact with aqueous medium (e.g. as comprised in the core or surrounding the nucleic acid-lipid particles.

In addition or alternatively, less than (or at most) 1, 2, 3, 4, 5, 6, 7, 8, 9, 10, 15, 20 wt. % of the one or more nucleic acid is exposed on (or adsorbed to) the outside of the nucleic acid-lipid particles.

In addition or alternatively, less than (or at most) 1, 2, 3, 4, 5, 6, 7, 8, 9, 10, 15, 20 wt. % of the one or more nucleic acid is comprised in the (aqueous) core of the nucleic acid-lipid particles.

The term “aqueous core” can refer to aqueous medium surrounded by the one or more lipid bilayer.

The term “encapsulation” can be understood as meaning that the at least part of the one or more nucleic acid (i.e. at least some (preferably all) molecules of the one or more nucleic acid) is enclosed (or trapped) within the outer boundary of the (anionic) nucleic-acid lipid particle. Preferably, part or all of the one or more nucleic acid (i.e. at least some (preferably all) molecules of the one or more nucleic acid) is not in (direct) contact with (aqueous) medium surrounding the (anionic) nucleic-acid lipid particle.

In particular, at least part of the one or more nucleic acid may be present in, or encompassed by, the one or more lipid bilayer. For example, provided is a composition comprising nucleic acid-lipid particles, wherein the nucleic acid-lipid particles comprise:
one or more nucleic acid;
one or more lipid bilayer comprising at least one anionic lipid and/or at least one neutral lipid;

wherein the nucleic acid-lipid particles are anionic, and wherein at least part of the one or more nucleic acid is present in the one or more lipid bilayer.

Preferably, at least 1, 2, 3, 4, 5, 6, 7, 8, 9, 10, 15, 20, 30, 40, 50, 60, 70, 80, 90 wt. % of the one or more nucleic acid in the nucleic acid-lipid particles is present in the one or more lipid bilayer, relative to total weight of the one or more nucleic acid of the nucleic acid-lipid particles. The composition preferably comprises between 1×10⁶-1×10¹²/ml of said particles.

The composition may be a liquid composition. Alternatively, the composition may be a solid composition e.g. is a powder composition (e.g. a composition comprising lyophilized nucleic acid-lipid particles). In addition or alternatively, the composition may be a frozen formulation.

The term “nucleic acid” as used herein means a polymer containing at least two nucleotides, preferably at least two deoxyribonucleotides and/or ribonucleotides, in either single-or double-stranded form. The term thus includes DNA, RNA and DNA/RNA hybrids. DNA may be in the form of e.g. antisense molecules, plasmid DNA, pre-condensed DNA, a PCR product, vectors (P1, PAC, BAC, YAC, artificial chromosomes), expression cassettes, chimeric sequences, chromosomal DNA, or derivatives and combinations of these groups. RNA may be in the form of siRNA, asymmetrical interfering RNA (aiRNA), microRNA (miRNA), lncRNA, gRNA, mRNA, tRNA, rRNA, tRNA, viral RNA (vRNA), and combinations thereof. The term “nucleic acid” includes nucleic acids containing nucleotide analogs or modified backbone residue(s) or linkage(s), which are synthetic, naturally occurring, or non-naturally occurring, and which may have similar or different binding properties as the reference nucleic acid not having said modifications or differences. Examples of such analogs include, without limitation, cyclic dinucleotides, phosphorothioates, phosphoramidates, methyl phosphonates, chiral-methyl phosphonates, 2′-O-methyl ribonucleotides, and peptide-nucleic acids (PNAs). Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)). “Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides. The “nucleic acid” of the current invention may be naturally occurring or not naturally occurring (i.e. a “nucleic acid analogue”). Accordingly, the term “nucleic acid” as used herein may be replaced by “nucleic acid and/or nucleic acid analogue”. The “nucleic acid analogue” can for example be a polymer of nucleotides (e.g. at least two nucleotides) having at least one alteration in one or more of the phosphate backbone, pentose sugar, and one of four nucleobases. The “nucleic acid analogue” can for example be synthetic, i.e. produced by chemical synthesis method. The “nucleic acid analogue” may be a molecule structurally similar to naturally occurring nucleic acid, but having an alteration in the backbone of the molecule, for example in the case of peptide nucleic acid (PNA), Morpholino and locked nucleic acid (LNA), as well as glycol nucleic acid (GNA), threose nucleic acid (TNA) and hexitol nucleic acids (HNA) and lipid nucleic acid. The term “RNA analogue” as used herein may for example refer to a nucleic acid analogue wherein the pentose sugar is ribose. The “RNA analogue” as used herein is preferably structurally similar to dsRNA. Where reference is made to “one or more” nucleic acid, this may refer to one or more different types or forms of nucleic acid, for example chosen from the above, or may refer to one or more nucleic acid with different constituent residues or different sequence. For example, one or more may refer to at least 1, 2, 3, 4, 5, 10 nucleic acid. Thus, the term “one nucleic acid” may refer to one (type of) nucleic acid which may be present as multiple (identical) nucleic acid molecules. The one or more nucleic acid may be non-coding nucleic acid.

Preferably, the one or more nucleic acid is comprised in the particles or in the one or more lipid bilayer thereof in the form of nucleic acid-lipid complexes, preferably stabilized by hydrophobic interaction(s) between lipid and nonpolar moieties of the nucleic acid. In addition or alternatively, the one or more nucleic acid is comprised/encapsulated in the particles or in the one or more lipid bilayer thereof, wherein the encapsulation is not or substantially not by electrostatic interaction(s).

The nucleic acid in the context of the current invention may have any length, for example at least 10, 100, 1000 bp (base pair), or at least 10, 1000 kb (kilobase), an/or no more than 1000, 10, 1 kb, but preferably has a length of 100 bp to 100 kb, preferably 0.1-20 kb.

In a preferred embodiment, the nucleic acid is an immunologic adjuvant. The term “immunologic adjuvant” in the context of the current invention means a substance or compound that modulates, preferably increases, the (innate or adaptive) immune response to another immunological agent, such as a vaccine and/or a cancer immunological agent. This typically means that the potency of the immunological agent is increased and/or less immunological agent is needed to achieve a similar efficacy of immune response, in comparison to when the immunologic adjuvant is not used. The “immunologic adjuvant” preferably mimics the activity of pathogen-associated molecular patterns (PAMPs), which include lipopolysaccharide, molecular cages for antigens, components of bacterial cell walls, and nucleic acids such as single-stranded RNA, double-stranded RNA, single-stranded DNA, unmethylated CpG dinucleotide-containing DNA or DNA-RNA hybrids. In addition or alternatively, the term “immunologic adjuvant” as used herein can mean a substance or compound that further enhances the immune response unspecifically or specifically through binding to pattern recognition receptors (PRRs) including but not limited to C-type lectin receptors (CLRs), RIG-like receptors (RLRs), nucleotide-binding oligomerization domain (NOD) proteins, stimulator of interferon genes (STING) and toll-like receptors (TLRs). Preferably, the present disclosure is not for use as SARS-Cov2 vaccine. Preferably, the nucleic-acid-lipid particles according to the present invention do not comprise a SARS-Cov2 antigen.

In a preferred embodiment, the immunologic adjuvant and/or the one or more nucleic acid is a TLR agonist, preferably an agonist for one or more of an endosomal TLR, more preferably TLR 3, 7, 8, and/or 9. The “toll-like-receptor” in the context of the current invention can be TLR1, TLR2, TLR3, TLR4, TLR5, TLR6, TLR7, TLR8, TLR9, TLR10, TLR11, TLR12, and TLR13, preferably TLR3. The immunological adjuvant and/or TLR agonist is preferably one or more CpG (cytosine followed by guanine residue), CpG motif, methylated CpG, CpG oligodeoxynucleotide (ODN, e.g. 5-25 bp)) (e.g. CpG 1018, TLR9 agonist), Poly (I:C) (i.e. polyinosinic: polycytidylic acid, TL3 agonist), poly (I:C12U) (TLR3 agonist), poly (G: C) (TL3 agonist), poly (A:U) (TLR3 agonist), poly (U) (TLR7/8 agonist). The CpG ODN may be a class A, class B or class C CpG ODN.

The term “CpG” as used herein means a nucleic acid (analogue) having a cytosine followed by a guanine linked by a phosphate bond in which the pyrimidine ring of the cytosine is unmethylated. The term “methylated CpG” as used herein means methylation of the cytosine on the pyrimidine ring, preferably occurring at the 5-position of the pyrimidine ring. The term “CpG motif” as used herein means a pattern of bases that includes an unmethylated central CpG surrounded by at least one base flanking (on the 3′ and the 5′ side of) the central CpG. The term “CpG ODN” as used herein means an oligodeoxynucleotide comprises one or more unmethylated CpG and preferably is least about ten nucleotides in length. The “CpG ODN” is preferably single-stranded. The entire CpG ODN can be unmethylated or portions may be unmethylated. In one embodiment, at least the C of the 5′ CG 3′ is unmethylated. The “CpG ODN” may be a class A, class B or class C CpG ODN. The skilled person is familiar with the difference classes of CpG ODNs and the different types of CpG ODN that may be used in context of the current invention (e.g. Zhang et al. Pharmaceutics. 2021 Dec. 28; 14 (1): 73.),

In a preferred embodiment, the “nucleic acid” in the context of the current invention is a TLR3 agonist. TLR3 interacts with robose-phosphate backbone of double stranded RNA and has no specific sequence requirement. Preferably the “nucleic acid” is one or more of poly (I:C) (i.e. polyinosinic: polycytidylic acid), and poly(I:C₁₂U), The “poly (I:C)” according to the present invention preferably comprises single-stranded polyinosinic acid (Poly I) and single-stranded polycytidylic acid (Poly C), that are preferably not associated by hydrogen bonding or covalent bonding at the time of administration as well as double-stranded or complexed Poly I/Poly C. In addition or alternatively, the “Poly (I:C)” is preferably a mismatched double-stranded RNA with one strand being a polymer of inosinic acid, the other a polymer of cytidylic acid. The “poly (I:C₁₂U)” is preferably a poly (I:C) with a U mismatch at every 12th base of the C strand. Poly (A:U) (Polyadenylic-polyuridylic acid) or poly (C:G) (Polycytidylic-guanylic acid could be used as an alternative analogue to poly (I:C) in the context of the current invention.

In an embodiment, the nuclei acid-lipid particle is preferably a liposome, more preferably an anionic liposome. The term “liposome” as used herein means an (aqueous) compartment enclosed within at least one phospholipid bilayer. The “liposome” is preferably a closed vesicle, formed by a lipid bilayer enclosing an aqueous compartment. Typically, water soluble compounds are entrapped within the aqueous phases/core of the liposomes opposed to lipophilic compounds, which are typically entrapped in the core/center of the lipid bilayer membranes. The term “liposome” as used herein encompasses unilamellar vesicles (UV) or small unilamellar vesicles (SUV) comprising a single lipid bilayer or multilamellar vesicles

(MLV) comprising two or more concentric bilayers each separated from the next by a layer of water. The nucleic-acid lipid particle or liposome as disclosed herein may have a substantially spherical form.

Preferably, the one or more lipid bilayer of the present particles is/are arranged at least for 30, 40, 50, 60, 70, 80, 90% in a lamellar phase structure. A person skilled in the art can characterize the assembly of the lipid phases and overall structure of the lipid particle using electron microscopy, nuclear magnetic resonance (NMR), small angle X-ray scattering (SAXS) and/or small angle neutron scattering (SANS).

The liposome and/or nucleic acid-lipid particles as disclosed herein preferably have a core-shell structure (with a lipid shell) and/or a core encapsulated by the one or more lipid bilayer, e.g. determined by small angle neutron scattering (SANS), and/or wherein the core comprises at least 30, 40, 50, 60, 70, 80, 90 vol. % aqueous medium/aqueous solvent, relative to total core volume. The aqueous medium/solvent content in the core compartment can be determined by small angle neutron scattering (SANS) solvent contrast variation technique, using the calculated neutron scattering length density (SLD) values of the components and their volume fraction (as explained in the Experimental section). In addition and/or alternatively, the aqueous compartment comprises at most 25, 20, 15, 10, 5, 4, 2, 1 wt. % nucleic acid, relative to total nucleic acid of the nucleic acid lipid particle.

In a preferred embodiment, the at least one anionic lipid and/or at least one neutral lipid as comprised in the one or more lipid bilayer, as disclosed herein is preferably a phospholipid or a sphingolipid.

The phospholipid content is preferably calculated following measurement of the phosphate content. The Rouser assay (Rouser et al. Lipids 5, 494-496 (1970)) is a preferred method of establishing the phosphate content. The Rouser assay is based on the hydrolysis of phospholipids into orthophosphate by incubation with concentrated perchloric acid during heating and subsequent spectrophotometric quantification of inorganic phosphate after reaction with (ammonium) molybdate and ascorbic acid in a heated water bath. A preferred protocol of conducting the Rouser assay is as follows:

Liposome sample dilutions and known amounts of a 0.5 mM KH₂PO₄ standard solution are transferred into clean glass tubes, and the solvent is completely evaporated using a heat block at 220° C. Subsequently, 0.3 mL of perchloric acid is added to each tube and placed in the heat block for 60 min or until the yellow color disappeared. When cooled down, 1 mL of water, 0.5 mL of molybdate solution and, subsequently, 0.5 mL of ascorbic acid solution are added and agitated on a vortex. The tubes are placed in a warm water bath for 5 min and then cooled down. Next, the absorbance of the samples and standard is measured at 797 nm, using a spectrophotometric microplate reader (BMG SPECTROstar Nano, De Meern, the Netherlands).

The term “lipid bilayer” as used herein can refer to a two-layered arrangement of lipid molecules. The two-layered arrangements may be at least partially enabled by amphiphilic lipids and accordingly contain a polar and an apolar region. The polar region typically consist of a phosphate group, an acidic group and/or tertiary or quaternary ammonium salts and can either have a net negative (anionic), neutral or positive (cationic) surface charge at physiological pH, depending on the composition of the lipid head groups. The apolar region typically consists of one or more fatty acid chains with at least 8 carbons and/or cholesterol. The lipids constituting the vesicular bilayer membranes may be organized such that the apolar hydrocarbon “tails” are oriented toward the center of the bilayer while the polar “heads” orient towards the in- and outside aqueous phase, respectively. If there is more than one lipid bilayer in the particle of the invention, each lipid bilayer is preferably composed of two lipid monolayers, each of which has a hydrophobic “tail” region and a hydrophilic polar “head” region. Where reference is made to lipid(s) herein, preferably lipid(s) capable of forming one or more lipid bilayers is meant. The term “one or more lipid bilayer” can refer to (at least) 1, 2, 3 lipid bilayer(s).

The term “anionic (lipid or particle)” as used herein means a compound (e.g. lipid or particle such as a liposome) with a net negative charge in aqueous medium at physiologically acceptable pH (preferably at pH 7.0-7.8, preferably at ~7.4), wherein a net negative charge preferably means a zeta potential of <−2 mV, preferably <−10 mV, more preferably <−50 mV e.g. as measured in the medium described below. In addition or alternatively, the term “anionic (particle)” as used herein can mean a particle (e.g. liposome) having at least 0.1 mol % (e.g. at least 1, 5, 10, 20, 30, 40, 50, or 60 mol %) of at least one anionic lipid relative to total mol lipid of the particle (e.g. liposome).

The present inventors found that an increase in anionic lipid proportion led to a more anionic surface. The inclusion of anionic lipid appears to play a role in enhancing colloid stability. Formulations without anionic lipid tend to aggregate after a few days of storage.

The term “anionic lipid” in the context of the current invention means a lipid with a negative net charge in aqueous solution at pH 3-9, preferably at pH 5-8, more preferably at pH 6.0-7.5, most preferably at pH 7.4. In the context of the current invention, the at least one anionic lipid can be one or more selected, but are not limited to, from the group consisting of: diacylglycerol phophatidic acid (1,2-distearoyl-sn-glycero-3-phosphate (DSPA); 1,2-dipalmitoyl-sn-glycero-3-phosphate (DPPA); 1,2-dimyristoyl-sn-glycero-3-phosphate (DMPA); 1,2-dilauroyl-sn-glycero-3-phosphate (DLPA); 1,2-dioleoyl-sn-glycero-3-phosphate (DOPA), diacylglycerol phosphoglycerol (1,2-distearoyl-sn-glycero-3-phospho-(1′-rac-glycerol) (DSPG); 1,2-dipalmitoyl-sn-glycero-3-phospho-(1′-rac-glycerol) (DPPG); 1,2-dimyristoyl-sn-glycero-3-phospho-(1′-rac-glycerol) (DMPG); 1,2-dilauroyl-sn-glycero-3-phospho-(1′-rac-glycerol) (DLPG); 1,2-dioleoyl-sn-glycero-3-phospho-(1′-rac-glycerol) (DOPG)), phosphatidylglycerol [e.g. egg hosphatidylglycerol (EPG)], cardiolipin, diacylphosphatidylinositol, diacylphosphatidylserine, N-succinyl phosphatidylethanolamine, N-glutarylphosphatidylethanolamine, lysylphosphatidylglycerol, gangliosides (e.g. GM1, GM2, GM3), sulphogycosphingolipids, fatty acids and anionic modifying groups joined to neutral lipids. Preferably the anionic lipid is EPG. Preferably, the nucleic acid-lipid particles according to the invention do not comprise DPPG, or at most 5, 4, 3, 2, 1, or 0.5 mol. %, relative to total mol lipid of the particles.

The term “neutral lipid” in the context of the current invention means a lipid which does not (substantially) carry a net charge in aqueous solution at pH 5-9, preferably at pH 6-8, more preferably at pH 6.5-7.5, most preferably at pH 7.4. In the context of the current invention, the neutral lipid can be one or more selected, but are not limited to, from the group consisting of:

phosphatidylcholine [e.g. egg phosphatidylcholine (EPC)], diacylglycerol phosphocholine (L-α-phosphatidylcholine, hydrogenated (Soy) (HSPC); diacylglycerol phosphocholine (L-α-phosphatidylcholine, (Soy) (Soy PC) 1,2-distearoyl-sn-glycero-3-phosphocholine (DSPC); 1,2-dipalmitoyl-sn-glycero-3-phosphocholine (DPPC); 1,2-dimyristoyl-sn-glycero-3-phosphocholine (DMPC); 1,2-dilauroyl-sn-glycero-3-phosphocholine (DLPC); 1,2-dioleoyl-sn-glycero-3-phosphocholine (DOPC), diacylglycerol phosphoethanolamine (1,2-distearoyl-sn-glycero-3-phosphoethanolamine (DSPE); 1,2-dipalmitoyl-sn-glycero-3-phosphoethanolamine (DPPE); 1,2-dimyristoyl-sn-glycero-3-phosphoethanolamine (DMPE); 1,2-dilauroyl-sn-glycero-3-phosphoethanolamine (DLPE); 1,2-dioleoyl-sn-glycero-3-phosphoethanolamine (DOPE), ceramide, sphingosines, sphingomyelin, cephalin, glycolipids like glycoglycerolipids (monogalactosyldiacylglycerol (MGD), digalactosyldiacylglycerol (DGD),

sulfoquinovosyldiacylglycerol (SQDG)) and glycosphingolipids (α-galactosylceramide, beta-mannoseceramide), cholesterol and other sterols and mono, di or triacylglycerols. Preferably the neutral lipid is EPC. In addition or alternatively, the at least one neutral lipid may involve a glycolipid which may aid immune system targeting.

The term “cationic lipid” in the context of the current invention means a lipid with a positive net charge in aqueous solution at pH 3-9, preferably at pH 5-8, more preferably at pH 6.0-7.5, most preferably at pH 7.4.

In the context of the current invention, the cationic lipid can be one or more selected, but not limited to, from the group consisting of:

N, N-dioleyl-N, N-dimethylammonium chloride (“DODAC”); N-(2,3-dioleyloxy) propyl)-N,N,N-trimethylammonium chloride (“DOTMA”); N-(2,3-dioleyloxy) propyl)-N, N-dimethylammonium chloride (“DODMA”); N,N-distearyl-N,N-dimethylammonium bromide (“DDAB”); N-(2,3-dioleoyloxy) propyl)-N,N, N-trimethylammonium chloride (“DOTAP”); N-(2,3-dioleoyloxy) propyl)-N, N-dimethylammonium chloride (“DODAP”); 3-(N-(N′,N′-dimethylaminoethane) carbamoyl) cholesterol (“DC-Chol”); N-(1,2-dimyristyloxyprop-3-yl)-N,N-dimethyl-N-hydroxyethyl ammonium bromide (“DMRIE”); 1.2-dilinoleyloxy-N, N-dimethyl-3-aminopropane (DLinDMA); 1,2-distearyloxy-N,N-dimethyl-3-aminopropane (DSDMA); 1.2-dilinolenyloxy-N, N-dimethyl-3-aminopropane (DLenDMA); 2-{4-[(3b)-cholest-5-en-3-yloxy]butoxy}-N,N-dimethyl-3-[(9Z,12Z)-octadeca-9, 12-dien-1-yloxy]propan-amine (CLinDMA).

In an embodiment, the (anionic or neutral) lipid as disclosed herein is a polysarcosine or polyoxazoline or a pegylated lipid (or PEGylated lipid or PEG lipid). A “pegylated lipid” as used herein means a lipid molecule comprising a lipid moiety with covalent and/or non-covalent attachment of one or more polyethylene glycol (i.e, PEG) polymer chains, wherein the PEG moiety has an average molecular weight of from about 2,000 to 20,000 daltons. The pegylated lipid may be one or more selected from the group consisting of:

1,2-distearoyl-sn-glycero-3-phosphoethanolamine-N-[methoxy (polyethylene glycol)-2000](mPEG-2000-DSPE); 1,2-dioctadecanoyl-sn-glycero-3-phosphoethanolamine-N-[methoxy (polyethylene glycol)-2000](mPEG-2000-DOPE); 1,2-dipalmitoyl-sn-glycero-3-phosphoethanolamine-N-[methoxy (polyethylene glycol)-2000](mPEG-2000-DPPE); 1,2-dimyristoyl-sn-glycero-3-phosphoethanolamine-N-[methoxy (polyethylene glycol)-2000](mPEG-2000-DMPE); 1,2-dilauroyl-sn-glycero-3-phosphoethanolamine-N-[methoxy (polyethylene glycol)-2000](mPEG-2000-DLPE); 1,2-distearoyl-sn-glycero-3-phosphoethanolamine-N-[methoxy (polyethylene glycol)-5000](mPEG-5000-DSPE); 1,2-dioctadecanoyl-sn-glycero-3-phosphoethanolamine-N-[methoxy (polyethylene glycol)-5000](mPEG-5000-DOPE); 1,2-dipalmitoyl-sn-glycero-3-phosphoethanolamine-N-[methoxy (polyethylene glycol)-5000](mPEG-5000-DPPE); 1,2-dimyristoyl-sn-glycero-3-phosphoethanolamine-N-[methoxy (polyethylene glycol)-5000](mPEG-5000-DMPE); 1,2-dilauroyl-sn-glycero-3-phosphoethanolamine-N-[methoxy (polyethylene glycol)-5000](mPEG-5000-DLPE). Preferably, the nucleic acid-lipid particles according to the invention do not comprise PEGylated lipid, or at most 5, 4, 3, 2, 1, or 0.5 mol. %, relative to total mol lipid of the particles.

Preferably, the at least one anionic/neutral lipid of the invention is not DOPG, DOPE or DSPG, to improve stability of the particles.

The nucleic acid-lipid particles as disclosed herein preferably comprises
at least 0.5 mol. %, preferably at least 5 mol. %, more preferably at least 15 mol. % of at least one anionic lipid, relative to total mol lipid of the particles; and/or
at least 1 mol. %, preferably at least 5 mol. %, more preferably at least 15 mol. % of at least one neutral lipid, relative to total mol lipid of the particles.

In an embodiment, the nucleic acid-lipid particles as disclosed herein comprise at least 10, 20, or 30, or 40, or 50, or 60, or 70, or 80, or 90, or 95, or 99 mol % or 100 mol % of at least one anionic lipid and/or neutral lipid, relative to total mol lipid of the particles.

In an embodiment, the one or more lipid bilayer of the nucleic acid-lipid particles as disclosed herein comprises at least 5, 10, 15, 20, 25, 30, 35, 40, 45, 50, 55, 60, 65, 70, 75, 80, 85, 90, 95 mol % of at least one anionic lipid and/or at least one neutral lipid relative to total mol lipid of the one or more lipid bilayer. In addition or alternatively, the one or more lipid bilayer of the nucleic acid-lipid particle as disclosed herein comprises no more than 95, 90, 85, 80, 75, 70, 65, 60, 55, 50, 45, 40, 35, 30, 25, 20, 15, 10, 5 mol % of at least one anionic lipid and/or at least one neutral lipid relative to total mol lipid of the one or more lipid bilayer.

In a preferred embodiment, the nucleic acid-lipid particle as disclosed herein comprise 0.5-80 mol %, preferably 10-40 mol %, more preferably 10-30 mol % of one or more anionic lipids relative to total mol lipid present in the particles.

In a preferred embodiment, the nucleic acid-lipid particle as disclosed herein comprise 1-99.5 mol %, preferably 20-90 mol %, more preferably 60-90 mol % of one or more neutral lipids, relative to total mol lipid present in the particles.

In an embodiment, the nucleic acid-lipid particles as disclosed herein comprise no more than 25, 20, 15, 10, 5, 4, 3, 2, 1, 0.5, 0 mol % of at least one cationic lipid, relative to total mol lipid of the particles.

Preferably, the nucleic acid-lipid particles as according the present disclosure:
do not contain cationic lipid and/or comprise at most 1, 2, 3, 4, 5 mol. % cationic lipid relative to total mol lipid of the particles;
do not contain cationic polymer and/or comprise at most 1, 2, 3, 4, 5 mol. % cationic polymer relative to total mol lipid of the particles; and/or
do not contain multivalent cation and/or comprise at most 1, 2, 3, 4, 5 mol. % multivalent cation relative to total mol lipid of the particles.

In an embodiment, at least 5, 10, 15, 20, 25, 30, 40, 50, 60, 70, 80, 90, 95, 99 wt. % (e.g. 100 wt. %) of the one or more nucleic acid in the nucleic acid-lipid particles is present in the one or more lipid bilayer, relative to the total weight of nucleic acid in the nucleic acid-lipid particles.

Presence of nucleic acid in the one or more lipid bilayer may be evident from absence of nucleic acid in the (aqueous) core. In addition or alternatively, presence of nucleic acid in the one or more lipid bilayer may change the neutron contrast of the liposome shell, the bilayer fluidity properties and/or the thermal properties of the nucleic acid. In the prior art it is not anticipated to have even one molecule of nucleic acid in the lipid bilayer of an anionic particle.

In an embodiment, the nucleic acid-lipid particles as disclosed herein comprise at least 5, 10, 15, 20, 25, 30, 35, 40, 45, 50, 55, 60, 65, 70, 75, 80, 85, 90, 95 mol % of at least one sterol relative to total mol lipid (including sterol) of the particles. In an embodiment, the nucleic acid-lipid particles as disclosed herein comprises no more than 95, 90, 85, 80, 75, 70, 65, 60, 55, 50, 45, 40, 35, 30, 25, 20, 15, 10, 5 mol % of at least one sterol relative to total of mol lipid (including sterol) of the particles.

In a preferred embodiment, the nucleic acid-lipid particles as disclosed herein comprise 5-50 mol %, preferably 10-40 mol %, more preferably 30-35 mol % sterol, relative to total of mol lipid (including sterol) of the particles.

The “sterol” as used herein preferably means a steroid which has a hydroxyl group at position C-3 and has a skeleton derived from cholestane. Herein, the term “a skeleton derived from cholestane” refers to a skeleton wherein an unsaturated bond is introduced into the cholestane skeleton. The sterol of the invention is preferably a cholesterol or a cholesterol derivative. Herein, “cholesterol derivative”, e.g. refers to at least one selected from the group consisting of cholesterol, sitosterol (e.g. p-sitosterol), ergosterol, stigmasterol, 4,22-stigmastadien-3-on, stigmasterol acetate, lanosterol, and cycloartenol, or any combination thereof. In addition or alternatively, preferred sterols in the context of the current invention can be one or more of ergosterol, ergocalciferolm, steroidal saponin, vitamin D, campesterol, desmosterol, beta.-cholestanol, and estradiol. The sterol can be derived from and/or naturally found in a plant and/or animal.

The present inventors found that enriching the nucleic acid lipid particles with cholesterol allows for lower polydispersity index (PDI) of the formulation and higher encapsulation efficiency. Without being bound by theory, cholesterol appears to play a role in stabilising lipid particles containing RNA. Cholesterol induces changes in the lipid packaging in the bilayers, promoting the formation of lipid domains. Nucleic acids induce the lipid segregation of anionic liposomes into neutral lipid-rich areas and anionic lipid-rich areas. Including cholesterol in the bilayer may stabilize lipid segregation, avoiding the electrostatic repulsion between RNA and anionic lipids in the bilayer.

Preferably, the particles according to the present disclosure comprise at most 1, 2, 3, 4, 5, 6, 7, 8, 9, 10, 11, 12, 13, 14, 15, 16, 17, 18, 19, 20 mol. % of cationic molecules, e.g. one or more of cationic lipid, cationic polymer, and multivalent cation, relative to total mol lipid of the particles.

In an embodiment, the nucleic acid-lipid particles in the context of the current invention are free or substantially free of cationic lipids. In an embodiment, the nucleic acid-lipid particles in the context of the current invention, or the one or more lipid bilayer thereof, comprise at most 1 mol %, preferably at most 0.1%, more preferably at most 0.01 mol %, even more preferably at most 0.001 mol %, most preferably 0 mol % of (at least one) cationic lipid, cationic polymer or multivalent cationic ion relative to the total mol lipid of the particles.

In an embodiment, the nucleic acid-lipid particles in the context of the current invention, or the one or more lipid bilayers thereof, are free of polymer (other than nucleic acid), preferably cationic polymer. In an embodiment, the nucleic acid-lipid particles in the context of the current invention, or the one or more lipid bilayer thereof, comprise at most 1 mol. %, preferably at most 0.1 mol. %, more preferably at most 0.01 mol. %, even more preferably at most 0.001 mol. %, most preferably at 0 mol. % polymer (other than nucleic acid), preferably cationic polymer, with respect to the total mol lipid of the particles.

The term “free of” as used herein in the context of a molecule, substance or compound in nucleic acid-lipid particles can mean that the nucleic acid-lipid particles are “essentially free of” said molecule, substance or compound, e.g. the amount is not measurable according to a standard analytical technique in the field. In addition or alternatively, “(essentially) free of” can mean an amount less than 0.001 wt. %, or less than 0.0001 wt. %, or less than 0.00001 wt. %, or less than 0.000001 wt. %.

The term “cationic polymer” as used herein means a polymer bearing a net positive charge, preferably in aqueous medium at pH 7.4 (or at pH 3-9, 5-8, 6.0-7.5). The “cationic polymer” is preferably one or more of poly-L-lysine, polyamidoamine, poly[2-(N, N-dimethylamino)ethylmethacrylate], chitosan, poly-L-ornithine, cyclodextrin, histone, Collagen, dextran, and polyethyleneimine (PEI).

The present inventors found that that fine-tuning of the lipid composition of the anionic liposomes can direct their immunological activity to achieve either predominantly immunostimulatory or immunosilent action.

In an embodiment, the particles and/or composition of the disclosure is immunostimulatory, meaning that it may enable the activation of nucleic acid sensing receptors (e.g. TLR, RLR, STING) and may induce the maturation of immune cells (e.g. macrophages, dendritic cells, T-cells).

In an embodiment, the particle and/or composition of the disclosure is immunosilent or immunotolerant, meaning that it may reduce the activation of nucleic acid sensing receptors (e.g. TLR, RLR, STING) and may avoid the maturation of immune cells (e.g. macrophages, dendritic cells, T-cells).

In a preferred embodiment, the nucleic acid-lipid particles in the composition as disclosed herein comprises 0.5-40 mol %, preferably 5-35 mol %, more preferably 10-30 mol %, of at least one anionic lipid, relative to total mol lipid of the particles. In this embodiment, the composition is preferably an immunostimulatory composition (leading to e.g. activation of dendritic cells, activation of T cells and/or secretion of IFN-alpha).

In a preferred embodiment, the nucleic acid-lipid particles in the composition as disclosed herein comprise 0.5-80 mol %, preferably 3-40 mol %, more preferably 5-30 mol %, of at least one anionic lipid, relative to total mol lipid of the particles. In this embodiment, the composition is preferably an immunosilent composition (does not result in immunostimulation like e.g. activation of dendritic cells, activation of T cells and/or secretion of IFN-alpha).

Preferably, the nucleic acid-lipid particles of the invention have a particle diameter or average particle diameter of 30-500 nm, preferably 50-300 nm, more preferably 50-250 nm, more preferably 75-200 nm, even more preferably 75-190, or 100-150 nm. In addition or alternatively, the nucleic acid-lipid particles of the invention have a particle diameter below 300, 290, 280, 270, 260, 250, 240, 230, 220, 210, 200, 199, 198, 197, 196, 195, 194, 193, 192, 191, 190, 180, 170 nm and/or above 20, 30, 40, 50, 60, 70, 80, 90, 100, 110, 120, 130, 140, 150, 160, 170, 180, 190 nm. In addition or alternatively, the nucleic acid-lipid particles are comprised in a composition comprising at least 2, 10, 100, 1000, 104, 105, 106, 107, 108 or more particles, such as per m1 (or per μl).

The “particle diameter” in the context of the current invention is preferably the hydrodynamic diameter, i.e. the diameter of a perfect solid sphere that would exhibit the same hydrodynamic friction as the particle. The (average) particle diameter can be measured by dynamic light scattering (DLS), e.g. using a Malvern Zetasizer Nano S (Malvern Instruments, Malvern, UK), such as equipped with a He—Ne 633 nm laser configured with a scattering angle of 173°. The “(average) hydrodynamic particle diameter” can refer to the diameter of a single particle or the average particle diameter as determined for a collection of particles as present e.g. in a composition.

The “polydispersity” (or polydispersity index, PDI) as used herein is a measure of the heterogeneity of a sample based on particle diameter. A preferred method of determining the hydrodynamic diameter and the polydispersity (PDI) is by measuring dynamic light scattering (DLS) e.g. using a Malvern Zetasizer Nano S (Malvern Instruments, Malvern, UK), such as equipped with a He—Ne 633 nm laser configured with a scattering angle of 173°. A preferred DLS protocol is as follows:

samples are diluted in PBS. DLS is performed using a Malvern Zetasizer Nano S (Malvern Instruments, Malvern, UK) equipped with a He—Ne 633 nm laser configured with a scattering angle of 173°. The measurements are performed using Malvern's Zetasizer v7.13 software (Malvern Instruments, Malvern, UK). A minimum of 3 measurements are performed per sample, and the measurement duration is automatically set by the software depending on the samples' characteristics. A viscosity of 0.8872 centipoises (cP) and refractive index (RI) of 1.330 for the dispersant and an RI of 1.590 and absorption of 0.010 for the material in suspension are set in the software of the instrument. The samples are diluted in PBS, and measured at 25° C. A PDI<0.3 is considered acceptable.

Preferably, the nucleic acid-lipid particles of the disclosure have a polydispersity index (i.e. PDI) in particle diameter of no more than 0.6, 0.5, 0.4, 0.35, 0.30, 0.25, 0.2, 0.15. In an embodiment, the nucleic acid-lipid particles of the disclosure have a polydispersity index (i.e. PDI) of 0.01-0.5, preferably 0.01-0.5, more preferably 0.01-0.4, more preferably 0.01-0.25.

In an embodiment, the nucleic acid-lipid particles of the disclosure have a zeta potential of between −100 and −10 mV, preferably between −200 and −5 mV, or between −150 and −10 mV, or between −100 and −15 mV, or between −50 and −20 mV. Exemplary measurement conditions are described below.

The term “zeta potential” as used herein is a measure of the overall charge that a particle or plurality of particles acquire(s) in a particular medium. The “zeta potential” can refer to the charge of a single particle or the (average) overall charge of a collection of particles as determined for a collection of particles. Generally, larger zeta potentials predict a more stable dispersion, which means that all the particles in suspension will tend to repel each other thus preventing aggregation. The zeta potential is preferably expressed in mV and can be determined by electrophoretic light scattering (ELS). A preferred protocol of measuring zeta potential by ELS is as follows:

samples are diluted in 0.1× PBS (1 volume of PBS and 9 volumes of purified water). The zeta potential is determined using Zetasizer Nano Z (Malvern Instruments, Malvern, UK) and Malvern's Zetasizer v7.13 software. A minimum of 3 measurements are performed per sample, and the measurement duration is automatically set by the software depending on the samples' characteristics. The samples are measured at 25° C. A viscosity of 0.8872 cP, RI of 1.330 and dielectric constant of 78.5 for the dispersant and an RI of 1.590 and absorption of 0.010 for the material in suspension are set in the software of the instrument.

In a preferred embodiment, the nucleic acid-lipid particles have
an average diameter of 50-250 nm;
a PDI of 0.01-0.3; and/or
a zeta potential between −100 and −10 mV.

In a preferred embodiment, the nucleic-acid lipid particles are non-inflammatory and/or non-immunogenic, which means that they do not (substantially) result in activation of dendritic cells, activation of T cells and/or secretion of IFN-alpha.

In certain embodiments, for example in case the one or more nucleic acid is one or more mRNA, the nucleic-acid lipid particles comprise endosomal escape peptides, preferably present at, or extending from, the surface of the nucleic-acid lipid particles.

In an embodiment, the composition is (administered) in combination with one or more selected from the group consisting of an antigen, a pathogen derived antigen, a tumor associated (or specific) antigen, a vaccine, an immunologic adjuvant, and an anti-neoplastic agent.

The anti-neoplastic agent may be one or more of:
a cell-based anti-neoplastic agent;
a lymphocyte-based anti-neoplastic agent, preferably chosen from B cells, αβT cells, γδ T cells, NK cells, NKT cells, autologous tumor-infiltrating lymphocytes (TILs), autologous NK cells, CAR-T cells, CAR-B cells, CAR-NK cells, CAR-NKT cells;
myeloid-based anti-neoplastic agent, preferably chosen from dendritic cell-based anti-neoplastic agent, macrophage-based anti-neoplastic agent or a neutrophil based anti-neoplastic agent.
an antibody, or an immune checkpoint inhibitor, wherein the immune checkpoint inhibitor is preferably an antibody, more preferably anti-CTLA4 antibody, anti-PD1 antibody and/or anti-PD-L1 antibody;
a small molecule drug preferably chosen from alkylating agent, antibiotic, anti-metabolite, hormonal antagonist, photosensitizer, protein kinase inhibitor, poly (ADP-ribose) polymerase inhibitor, taxane and/or topoisomerase inhibitor.
radiation therapy;
a cytokine preferably chosen from IL-2, IL-12, IL-15 and IL-21;
growth factor preferably chosen from CSF family, and FIt3L; and/or
a steroidal or a non-steroidal anti-inflammatory drug.

The term “in combination with” as used herein does not exclude the possibly that the individual components in combination are not in one physical entity (and in a single formulation), meaning that the individual components in combination can for example be administered simultaneously, sequentially or separately. However, the combination may be co-encapsulated in the same particle as disclosed herein. In certain embodiments of the current invention, a joint effect may justify the components in combination (for example the nucleic acid lipid particle and the anti-neoplastic agent) in a single formulation. In other embodiments, a joint effect may justify the components in combination in separate formulations. In yet other embodiments, a joint effect may justify the components in combination with a different timing and/or a different route of administration. The components in combination may be presented side-by-side (e.g. to a human or animal subject) so that they can be applied simultaneously, separately or at intervals to said subject. In addition or alternatively, the components in combination may be separately packed in the form of a “kit-of-parts”. In addition or alternatively, the optimal administration scheme of the components in combination may be determined on a case-to-case basis, for example depending on the subject to be treated, depending on the condition or disease to be treated, depending on the route of administration, depending on the formulation, and dose.

The term “vaccine” in the context of the current invention particularly means a preparation that contains one or more antigens, e.g. used for the purpose of the prevention or treatment of infections, cancer, auto-immune disease or allergy.

The “anti-neoplastic agent” may refer to radiotherapeutic agent, chemotherapeutic agent, an antibody, T-cell receptor or immune cell, such as a CAR T-cell, or may refer to for example an immune checkpoint inhibitor, such as an anti-CTLA-4, an anti-PD1 antibody and/or anti-PD-L1 antibody. The immune cell may be an immune cell administered as part of immunotherapy. An “immune cell” according to the present disclosure may be any cell belonging to the immune system, preferably chosen from a lymphocyte, granulocyte, myeloid cell, T cell (e.g. T helper cell, T helper 17 cell, follicular helper T cell, cytotoxic T cell, gamma delta T cell), monocyte, macrophage, NK cell, basophil, dendritic cell (e.g. myeloid dendritic cell, plasmacytoid dendritic cell), neutrophil, eosinophil, basophil, mast cell, B cell, or plasma cell, among others. An “immune cell” according to the present disclosure may also be an engineered immune cell as taught herein. In a preferred embodiment, the immune cell according to the present disclosure is an engineered cytotoxic T cell or NK cell such as a chimeric antigen receptor (CAR) T or CAR NK cell.

### Use of the Invention

In a preferred embodiment, the nucleic acid-lipid particle and/or the composition as disclosed herein is for use in preventing and/or treating a disease. In other words, the present invention also involves a method for preventing or treating a disease, comprising administering the composition as disclosed herein to a subject (an animal, preferably a human) in need thereof. Administration is preferably by oral, topical (including dermal, buccal and sublingual), rectal,, parenteral (intradermal, intramuscular, subcutaneous, or intravenous), nasal and pulmonary, more preferably by parenteral administration, most preferably by intravenous administration . . .

The term “preventing” or “prevention” in the context of the current invention means to reduce the chance that a subject develops a condition or disease. Preventing also encompasses a situation wherein a condition or disease is delayed, reduced in severity and/or reduced in incidence, even when the condition or disease is not entirely circumvented. In the context of the current invention, the terms “preventing” or “prevention” encompasses the situation wherein a subject previously has experienced a condition or disease but an intervention keeps the condition from recurring. The terms “preventing” or “prevention” may have a therapeutic and/or a non-therapeutic effect, but preferably has a therapeutic effect. If the “preventing” or “prevention” is therapeutic in nature, it may be also directed at a symptom of a disease or condition and/or an underlying pathology thereof. The terms “preventing”, or “prevention” can be defined by any delay, change in severity, and/or change in incidence, such as of at least 5, 10, 20, 30, 40, 50, 60, 70, 80, 90, 100%, as compared to a control or reference as measured by any standard technique.

The term “treating” or “treatment” in the context of the current invention means that an intervention reduces and/or cures a condition or disease once the condition or disease is already existing. The “treating” or “treatment” may have a therapeutic and/or a non-therapeutic effect, but preferably has a therapeutic effect. If the “treating” or “treatment” is therapeutic in nature, it may be directed at a symptom of a disease or condition and/or an underlying pathology thereof. The treatment can for example be any reduction in severity, incidence, and/or frequency of the condition or disease, such as of at least 5%, 10%, 20%, 40%, 50%, 60%, 80%, 90%, 95%, or completely (100%), as compared to a control or reference as measured by any standard technique.

The disease may be one or more of cancer, infection, chronic inflammation, genetic disease and autoimmune disease. In an embodiment, the disease is cancer, for example biliary tract cancer, brain cancer (e.g. including glioblastomas and medulloblastomas), breast cancer (e.g. including inflammatory breast cancer), cervical cancer, choriocarcinoma, colon cancer, endometrial cancer, esophageal cancer, gastric cancer, hematological neoplasms (e.g. including acute lymphocytic and myelogenous leukemia), multiple myeloma, AIDS associated leukemias and adult T-cell leukemia lymphoma, intraepithelial neoplasms (e.g. including Bowen's disease and Paget's disease), liver cancer (hepatocarcinoma), lung cancer, lymphomas (e.g. including Hodgkin's disease and lymphocytic lymphomas), neuroblastomas, oral cancer (e.g. including squamous cell carcinoma), ovarian cancer (e.g. including those arising from epithelial cells, stromal cells, germ cells and mesenchymal cells), pancreas cancer, prostate cancer, rectal cancer, sarcomas (e.g. including leiomyosarcoma, rhabdomyosarcoma, liposarcoma, fibrosarcoma and osteosarcoma), skin cancer (e.g. including melanoma, Kaposi's sarcoma, basocellular cancer and squamous cell cancer), testicular cancer (e.g. including germinal tumours, seminoma, non-seminoma [teratomas, choriocarcinomas]), stromal tumours and germ cell tumours, thyroid cancer (e.g. including thyroid adenocarcinoma and medullar carcinoma), and renal cancer (e.g. including adenocarcinoma and Wilms tumour). In a preferred embodiment, the composition is administered to a patient with a prior diagnosis of cancer which has been resected. In many cases, like in melanoma, part of the tumor tissue may have been removed.

In an embodiment, the disease is a hyperproliferative or differentiative disorder, such as fibrosis or hyperplasia, such as pulmonary fibrosis or hyperplasia (e.g. benign prostatic hyperplasia), cardiac fibrosis, or liver fibrosis.

In an embodiment, the disease is an infection, such as a bacterial, viral or fungal infection, such as but not limited to human immunodeficiency virus (HIV) infection, hepatitis C virus (HCV) infection, hepatitis B infections (HBV), or cytomegalovirus (CMV) infection. The use of the composition in preventing or treating an infection encompasses the use of the composition as monotherapy and/or in combination with a pathogen-derived antigen or vaccine. For example, the composition may be administered sequentially to or simultaneously with a vaccine in the prevention or treatment of an infection.

In an embodiment, the disease is (chronic) inflammation, such as prostatitis, vernal keratoconjunctivitis, atherosclerosis, or idiopathic pulmonary pneumonia.

In an embodiment, the disease is an autoimmune disease or (chronic) inflammatory condition, such as systemic lupus erythematosus (SLE), multiple sclerosis (MS), pemphigus vulgaris (PV), or myasthenia gravis.

In a preferred embodiment, the use of the nucleic acid-lipid particle and/or the composition as disclosed (e.g. in cancer) is in combination with an immunotherapy, preferably wherein the immunotherapy involves administration of an antibody such as an immune checkpoint inhibitor or an immune cell based immunotherapy such as DC-cell therapy, CAR T-cell-therapy or NK-cell therapy.

The immune checkpoint inhibitor may be an inhibitor of one or more of CTLA-4, LAG-3, Tim3 VISTA, CD137, 0X40, ID01 PD-1, and PD-L1, preferably PD-1, and PD-L1.

In addition or alternatively, the immune checkpoint inhibitor may be one or more selected from the group consisting of ipilimumab, pembrolizumab, nivolumab, cemiplimab, pidilizumab, atezolizumab, avelumab, durvalumab, BMS 936559, JNJ 61610588, urelumab, 9B12, PF-04518600, BMS-986016, TSR-022, MBG453, MEDI6469, MEDI6383, epacadostat, anti-PD1, and anti-PDL1, preferably anti-PD1 and/or anti-PDL1.

### Method of the Invention

The present invention also pertains to a method for preparing a composition comprising nucleic acid-lipid particles as disclosed herein, the method comprising:
a) providing one or more organic solution comprising one or more lipid, preferably at least one anionic lipid and/or at least one neutral lipid;
b) providing one or more aqueous solution comprising one or more nucleic acid;
c) combining the one or more organic solution and the one or more aqueous solution, thereby producing the composition comprising nucleic acid-lipid particles as disclosed herein.

Preferably, the combining in step c) refers to mixing the one or more organic solution and the one or more aqueous solution (substantially) in laminar flow, preferably in a microfluidics device. The combining step may be possible with other methods such as but not limited to solvent injection, pulsed jet flow method, depressurization of an expanded liquid organic solution into aqueous suspension (DELOS) method, supercritical anti-solvent (SAS) method, or film-hydration method.

In a preferred embodiment, one or more of steps a)-c) of the method are performed by a microfluidic device. The term “microfluidic device” as used herein means a device comprising a combination of two or more (micro-) channels, preferably etched and/or molded into a material (e.g. glass, silicon or polymer), wherein at least two (micro-) channels are connected together in order to achieve a desired feature (e.g. mixing, pumping, sorting), preferably to achieve a laminar flow and mixing of fluids. Preferably, the method for preparing a composition comprising nucleic acid-lipid particles according to the disclosure does not apply purification using dialysis.

In an embodiment, the combining or mixing in step c) is in a (substantially) laminar flow and/or the mixing of the aqueous and organic solutions in step c) of the method is laminar mixing. The mixing is preferably effected by passive diffusion of molecules from a high concentration to a lower concentration domain in laminar flow. As used herein, the term “laminar flow” means that the fluids follow smooth paths in layers, with each layer moving smoothly past the adjacent layers. The “laminar flow” is preferably defined by a Reynolds number of 2000 or less (e.g. 1900, 1800, 1700, 1600, 1500, 1400, 1300, 1200, 1100, 1000 or less), more preferably in conjunction with a smooth flow (e.g. not inducing chaotic advection, chaotic mixing, and/or lateral mixing). The Reynolds is preferably determined by considering the flow as a flow through a pipe, wherein the Reynolds number is defined as:

DH is the hydraulic diameter of the channel (m), Q is the volumetric flow rate (m3/s), A is the pipe's cross-sectional area (m²), u is the mean speed of the fluid (SI units: m/s), μ is the dynamic viscosity of the fluid (Pa·s=N·s/m2=kg/(m·s)), v is the kinematic viscosity of the fluid (v=μ/ρ (m²/s)), p is the density of the fluid (kg/m³).

The present inventors found that better encapsulation efficiency and better PDI could be achieved with laminar mixing, as compared to turbulent of chaotic mixing. Higher encapsulation efficiency and lower PDI is particularly achieved in laminar mixing and having the combination of at least one organic solution and two sequential aqueous solutions.

In an embodiment, the present disclosure does not exclude that step c) employs chaotic flow mixing and/or the mixing of the aqueous and organic solutions in step c) of the method under chaotic flow. The term “chaotic flow” as used herein means a flow that is not a laminar flow, and is neither constant in time nor presenting any regular periodicity. A turbulent flow can be an example of a chaotic flow. “Chaotic mixing” as used herein means that mixing occurs under chaotic flow.

In an embodiment, the present disclosure does not exclude that the flow in step c) is a turbulent flow and/or the mixing of the aqueous and organic solutions in step c) of the method is turbulent mixing. “Turbulent mixing” as used herein means that mixing occurs under a turbulent flow. The term “turbulent flow” as used herein means a fluid motion characterized by fluid flow not in parallel layers and/or with disruption between those layers. A “turbulent flow” is preferably dominated by inertial forces, which typically produce chaotic eddies, vortices and other flow instabilities. A “turbulent flow” preferably means that the flow is characterized by chaotic changes in pressure and flow velocity. In an embodiment, the term “turbulent flow” encompasses any type of flow that is not a laminar flow. In an embodiment, the turbulent flow is defined by a Reynolds number larger than 4000, preferably larger than 5000, more preferably larger than 6000. A turbulent flow can be an example of a chaotic flow.

Typically, a laminar flow is interspersed with turbulent flow until at a Reynolds number of 2000-4000, or 2500-3500. A flow of between 2000-4000, or 2500-3500 may herein be defined as a laminar and/or turbulent flow.

In an embodiment, chaotic and/or turbulent mixing (or chaotic and/or turbulent flow) according to the invention is achieved by providing a mixer device, preferably a micromixer chip on a microfluidic device. The mixer device may achieve a chaotic and/or turbulent flow by mixing of a laminar flow.

In an embodiment, the solvent selected for the dissolution of the lipids to produce the particles, e.g. liposomes containing the one or more nucleic acid is one or more selected preferentially but not limited from the group of aqueous miscible solvents, such as methanol, ethanol, isopropanol, butanol, acetonitrile, acetone, dimethyl sulfoxide. More preferably, the organic solvent is an aqueous miscible solvent classified as a class 3 solvent. Class 3 includes no solvent known as a human health hazard at levels normally accepted in pharmaceuticals (permissible dose accepted of 50 mg or more per day) (Q3C (R6): Impurities: guideline for residual solvents EMA/CHMP/ICH/82260/2006). Examples of class 3 solvents are ethanol, acetone, dimethyl sulfoxide, isopropanol, ethyl ether, methyl acetate, 1-pentanol, 1-propanol. Most preferably, the organic solvent(s) is/are ethanol, isopropanol, or a mixture of them. The ethanol solution may comprise at least 10, 20, 30, 40, 50, 60, 70, 80, 90, 95, or 99% (v/v) ethanol. The ethanol solution may be pure ethanol (100% ethanol). In addition or alternatively, the ethanol solution may comprise no more than 99, 95, 90, 80, 70, 60, or 50% (v/v) ethanol. The isopropanol solution may comprise at least 10, 20, 30, 40, 50, 60, 70, 80, 90, 95, or 99% (v/v) isopropanol. The isopropanol solution may be pure isopropanol (100% isopropanol). In addition or alternatively, the isopropanol solution may comprise no more than 99, 95, 90, 80, 70, 60, or 50% (v/v) isopropanol.

The present inventors found that it is beneficial to use ethanol as organic solution. Furthermore, the present inventors found that an increase in ethanol concentration may led to larger and more monodisperse particles. Without being bound by theory, hydrophobic interactions may determine the mechanism for the encapsulation of nucleic acids in the nucleic acid lipid particles. In the presence of ethanol, nucleic acids change its conformation, exposing hydrophobic nucleotide bases that can interact with lipid tails, allowing the formation of liposomes containing the nucleic acids in the bilayer(s).

In an embodiment, the aqueous solution in the context of the current invention is water. In an embodiment, the aqueous solution in the context of the current invention contains an osmolar agent such as NaCl or sucrose and/or a pH buffer group, such as one or more selected but no limited from the group consisting of phosphate, histidine, HEPES, Tris, acetate, carbonate, and citrate. In addition or alternatively, the aqueous solution as disclosed herein is RNase and DNase free.

In a preferred embodiment, the aqueous solution as disclosed herein has a pH of between 5.0-9.5-preferably 5.5-9.0, more preferably 6.0-8.5, even more preferably 6.0-8.0.

In a preferred embodiment, the flow rate ratio (A: B) between the one or more aqueous solutions (A) and the one or more organic solutions (B) is between 48:1 and 1:10, preferably 24:1 and 1:8, more preferably 18:1 and 1:3, even more preferably 10:1 and 1:1.

In a preferred embodiment, the at least one anionic and/or at least one neutral lipid is provided in the organic solution disclosed herein in a concentration of 0.01-500 mM, preferably 0.1-50 mM, more 1-20 mM.

In a preferred embodiment, the one or more nucleic acid is provided in the aqueous solution in a concentration of 5-50000 μg/ml, preferably 10-50000 μg/ml, more preferably 50-5000 μg/ml, more preferably 100-2000 μg/ml, even more preferably 200-1000 μg/ml.

In a preferred embodiment, the organic solution in step a) comprises at least 10 mol %, preferably at least 20 mol %, more preferably at least 30 mol %, even more preferably at least 40 mol %, most preferably at least 50 mol % (e.g. at least 60, 70, 80, 90, 95, or 99 mol %) of at least one anionic and/or at least one neutral lipid, relative to total mol lipid in the organic solution.

In an embodiment, the organic solution comprises 5-50 mol %, preferably 10-40 mol %, more preferably 20-30 mol % anionic lipid and/or neutral lipid, relative to total mol lipid in the organic solution.

In a preferred embodiment, the organic solution in step a) comprises at most 40 mol %, preferably at most 30 mol %, more preferably at most 20 mol %, even more preferably at most 10 mol %, most preferably at most 5 mol % (e.g. at most 4, 3, 2, 1, 0.5, or 0.1 mol %) of (at least one) cationic lipid, relative to total mol lipid in the organic solution.

In a preferred embodiment, the organic solution in step a) comprises at least 1 mol %, preferably at least 5 mol %, more preferably at least 10 mol %, even more preferably at least 20 mol %, most preferably at least 30 mol % of at least one sterol, relative to the total mol lipid (including sterol) in the organic solution. In an embodiment, the organic solution comprises 5-66 mol %, preferably 10-40 mol %, more preferably 20-35 mol % sterol, relative to total mol lipid (including sterol) in the organic solution.

In a preferred embodiment, the organic solution in step a) is free of cationic lipids and/or comprises at most 1 mol % of (at least one) cationic lipid relative to the total mol lipid in the organic solution.

In a preferred embodiment, the organic solution in step a) is free of cationic polymer, and/or comprises at most 1 mol. %, preferably at most 0.1 mol. %, more preferably at most 0.01 mol. % of at least one (cationic) polymer, calculated on the total mol lipid of the organic solution.

In a preferred embodiment, the method and/or particles of the invention do not involve or comprise multivalent cation (such as Ca²⁺, Mn²⁺, Mg²⁺) cationic lipid and/or a (cationic) polymer or at most 10, 5, 1 mol. % relative to total mol lipid of the particles.

In an embodiment, the method of the invention provides (or achieves) a nucleic acid encapsulation efficiency in the nucleic acid-lipid particles of at least 40%, preferably at least 60%, more preferably at least 80%, even more preferably at least 90%, most preferably at least 95%. In addition or alternatively, the method of the invention preferably provides (or achieves) a nucleic acid encapsulation efficiency in the nucleic acid-lipid particles of 60-95%, preferably 70-90%, more preferably 75-85%. The “encapsulation efficiency (of nucleic acid)” as used herein means the percentage of nucleic acid that is incorporated into the nucleic acid-lipid particle, when compared with the total amount of nucleic acid present in the formulation, and is calculated as follows:

$$Encapsulation efficiency ( \% ) = ( \frac{A ctual nucleic acid concentration ( \frac{μ g}{m L} )}{\frac{Intial nucleic acid mass ( μg )}{F inal formulation volume ( mL )}} ) \times 100 \%$$

### General Definitions

Reference to an element by the indefinite article ‘a’ or ‘an’ does not exclude the possibility that more than one of the elements is present, unless the context clearly requires that there be one and only one of the elements. The indefinite article ‘a’ or ‘an’ thus usually means ‘at least one’.

The terms ‘comprising’ or ‘to comprise’ and their conjugations, as used herein, refer to a situation wherein said terms are used in their non-limiting sense to mean that items following the word are included, but items not specifically mentioned are not excluded. It also encompasses the more limiting verb ‘to consist essentially of’ and ‘to consist of’.

### BRIEF DESCRIPTION OF FIGURES

FIG. 1: Set up of microfluidics chips employed for RNA (poly (I:C)) encapsulation in anionic liposomes. (A and B). In Design A, lipids dissolved in ethanol (yellow) flow in the central channel of a microfluidic chip while the RNA dissolved in RNase-free purified water (light blue) flows in the external channels. A co-flowing mixing induces the formation of RNA liposomes. In Design B, two chips are combined for a two-step encapsulation process. In Chip N° 1, RNA (poly (I:C)) dissolved in RNase-free purified water (light blue) flows in the central side of the first mixing channel while lipids dissolved in ethanol (yellow) flow in the external side of the first mixing channel. A co-flowing mixing occurs, and the resulting mixture enters Chip N°2. The flow rate of the additional water flows (dark blue) influences liposome characteristics. The five layers of flowing particles are mixed in a laminar flow, inducing the formation of RNA liposomes. Later, samples are collected from an output tube and diluted with RNase-free purified water to an ethanol concentration at least <20%. Ethanol and free RNA is removed using a tangential or centrifugal filtration technique.

FIG. 2: Application of design of experiment (DoE) approach for the development of a microfluidic process for the encapsulation of RNA in anionic liposomes using Design A or B. (I) Flow rates, lipid and RNA concentration in the solvents were the variables selected in the DoE's. (II) mean particle size, polydispersity (Pdl), and RNA encapsulation efficiency of the RNA-anionic liposomes were studied in this DoE. (III) A central composite design was selected as the experimental design for building a response surface model (RSM). (IV) The DoE was designed using appropriate software (Design-Expert™). (V) The DoE was executed for design A (n=30), Design B (n=53) and the data obtained was analysed, allowing the building of RSMs. (VI) Response surface models were used to predict the optimal process parameters. (VII) 5 independent batch were produced for the model validation. The grey area is an indication of the predicted interval by the RSMs of mean size, PDI and encapsulation efficiency. (Mean of three analytical replicates±SEM).

FIG. 3: Impact of mixing geometry and flow rate for mean size (A), polydispersity (B) and encapsulation efficiency (C). 500 μg/mL of RNA dissolved in RNase free water was mixed with an organic solution containing lipids (5 mM) [EPC: EPG: Cholesterol (3:1:2 molar ratio)] at flow rate ratio 1:1 and 0.25, 0.75 or 1.5 m¹/min total flow rate employing laminar and chaotic mixing microfluidics chips following the Design B scheme. Errors bars represent the standard deviation calculated from analytical triplicates from one independent experiment.

FIG. 4: Physical characterization of encapsulated RNA and size stability of anionic liposomes [EPC: EPG: Cholesterol (3:1:2)]containing RNA under incubation with human plasma. Encapsulated poly (I:C) was extracted from anionic liposomes by precipitation with isopropanol 100%. The clean pellet of RNA was resuspended in PBS and injected in a size exclusion chromatography (SEC) column (TSKgel® G-DNA-PW) running in PBS (flow rate=0.5 mL/min, 25° C.). Additionally, free poly (I:C) and standard DNA ruler (Gene Ruler 1 kb plus, Thermoscientific) samples were treated similarly and injected into the SEC column. A UV detector at λ=260 nm was used for RNA detection (A) and a multi-angle light scattering detector for molecular weight determination (B). Molecular weight analysis confirms that the poly (I:C) employed is a polymer with wide molecular weight distribution. Encapsulated poly (I:C) has a similar retention time (A) and molecular weight as compared to free poly (I:C), indicating that microfluidics technology can encapsulate nucleic acids without changing the original molecular weight distribution. The stability of empty and RNA-containing anionic liposomes in human plasma was studied using asymmetrical flow field-flow fractionation (AF4). Liposomes (2 mM of lipids) were incubated with PBS or 20% human plasma at 37° C. for 2 hours and then analysed by AF4. The radius of gyration (Rg) and light scattering intensity of the particles were determined using MALS (C), and protein corona formation was determined using a fluorescence detector (λₑₘ=280 nm, λₑₓ=340 nm) (D). Empty and RNA containing anionic liposomes do not showed particle aggregation in human plasma, as required for IV injection. Anionic liposomes protect encapsulated poly (I:C) from RNA degradation. Free and encapsulated poly (I:C) were incubated with or without RNase in reaction buffer or reaction buffer supplemented with human plasma (20% v/v) at 37° C. for 2 hours. Later, samples were run on a 1.0% agarose gel (E).

FIG. 5: TEM images of empty (A) and RNA containing (B) anionic liposomes [EPC: EPG: Cholesterol (3:1:2)] show the conservation of lipid bilayers after incorporating nucleic acids. However, the contrast of the nanoparticles changed after nucleic acids encapsulation. Uranyl salts stain phosphate groups of phospholipids and RNA molecules, and was used to increase particle contrast. 10 μL of diluted particle suspension (2 mM phospholipids) were placed in a previous glow discharged Formvar/Carbon coated copper grid and contrasted with uranyl oxalate (pH 7), and then contrasted-embedded in a mixture of 2% methyl cellulose/4% uranyl acetate (pH 4). Later, images were taken using a Tecnai 12 TEM microscope,

FIG. 6: Empty and poly(I:C)-containing anionic liposomes (Empty AL and pIC-AL) share a shell-core sphere structure (A, B). The shell is composed of lipids, while the core is aqueous. Poly (I:C) containing anionic liposomes have a higher scattering neutron density (SLD) in the shell than empty liposomes. Additionally, the scattering profile of the RNA containing liposomes does not change at 68% D2O, the contrast match for soluble RNA. This suggests that the RNA is embedded in the liposome bilayers rather than soluble in the aqueous core. Furthermore, the encapsulation of poly (I:C) changes the fluidity properties of the bilayers at temperatures higher than 40° C., an indication of the presence of RNA within the hydrophobic bilayer (C). Additionally, RNA containing anionic liposomes, but not empty liposomes have an exothermic event at 74° C., suggesting that poly (I:C) crystallizes when it is encapsulated in the liposomal bilayers (D). (A) SANS data (symbols) for empty (open grey rectangles) and poly (I:C) (open black circles) containing liposomes resuspended in 27%, 50%, 68% and 100% D20 water. The solid lines correspond to the best fit using the sphere core-shell model.

(B) Scattering length density (SLD) profiles as a function of the distance to the centre of the liposomes corresponding to the fits of the data in A. (C) General polarization measurements in a temperature interval from 10 to 80° C. (0,1° C. change per measurement) of empty anionic liposomes, anionic liposomes incubated with free poly (I:C) and poly(I:C) containing anionic liposomes. (D) Differential scanning calorimetry measurements in a temperature interval from 10 to 100° C. (0,5° C. change per minute) of free poly (I:C), empty anionic liposomes, empty anionic liposomes incubated with free poly (I:C) and poly(I:C) containing anionic liposomes.

FIG. 7: Stability studies of empty and poly(I:C) containing anionic liposomes [EPC: EPG: Cholesterol (3:1:2)]. Anionic liposomes in 10% sucrose 20 mM citrate buffer (pH=6.5) were stored for 5 months at −20° C. and 5° C. Particle mean size (A), polydispersity (B), zeta potential (C), poly (I:C) content (D) and % of poly (I:C) released from liposomes (E) and poly (I:C) in vitro activity (F) was determined at times 0 (shared characterization for both storage conditions) and at month 1 and month 5. (Mean of three analytical replicates±SEM).

FIG. 8: Poly (I:C) encapsulation in anionic liposomes [EPC: EPG: Cholesterol (3:1:2)] induces superior spleen conventional type 1 dendritic cell (cDC1) maturation ex vivo compared to soluble poly (I:C). A single cell suspension of splenocytes was incubated with 0.5 μg of poly (I:C) encapsulated in liposomes or an increasing amount of soluble poly (I:C) (1, 10 and 100 μg) (100 μL per well). After incubation for 45 minutes at 37° C., splenocytes were extensively washed, and cells were incubated for 15 h at 37° C., after which the cells were stained. The expression of CD80, CD86 and MHC II were determined by flow cytometry analysis. Indicated is the geometric mean fluorescent intensity (GMFI) averaged±SEM (n=5) for CD80 (A), CD86 (B) and MHC-II (C) expression by conventional type 1 dendritic cells. * p<0.05, ** p<0.01, **** p<0.0001

FIG. 9: Intravenous administration of poly (I:C)-containing anionic liposomes (pIC-AL) into mice boosts poly (I:C) immunostimulant effects in vivo without inducing acute hepatic toxicity. 10 or 50 μg of soluble or anionic liposomal poly (I:C) [EPC: EPG: Cholesterol (3:1:2)] were administered IV into mice. Blood samples were taken 3 hours of after IV injection for cytokines measurements. After 24 hours, mice were sacrificed, and blood was collected for cytokines determination and hepatic enzymatic (AST, ALT) activity determination. Additionally, the spleens were collected, and a single cell suspension was used for flow cytometry staining and analysis. Indicated are representative geometric mean fluorescence intensity (GMFI) mean±SEM of maturation markers on cDC1 (A, B, C) respectively [n=4]. Indicated are serum anti-viral cytokine fold enhancement (logarithmic transformed) and concentration after 3 and 24 hours of IV injection (D, E). Indicated are mean enzymatic activity (UI/L)±SEM in serum of hepatic enzymes ALT (F) and AST (G). * p<0.05, ** p<0.01, *** p<0.005 and **** p<0.0001.

FIG. 10: Intravenous administration of poly (I:C)-containing anionic liposomes (pIC-AL) but not of free poly (I:C) significantly augments vaccine effectiveness against the OVA₂₄₇₋₂₇₉ model antigen. Anionic liposomes [EPC: EPG: Cholesterol (3:1:2)] containing OVA₂₄₇₋₂₇₉ long peptide was administered IV into mice (22.5 nmol of phospholipid, 1,25 μg of peptide) alone or supplemented with soluble poly (I:C) (10 or 100 μg) or encapsulated poly (I:C) (10 ug) in anionic liposomes [EPC: EPG: Cholesterol (3:1:2)] (n=4 per group). On Day 7, H-2K^{b}/SIINFEKL tetramer binding T cells were identified (A). Splenocytes were restimulated ex vivo with SIINFEKL peptide for 5 h or 25 h, for CD8+ or CD4+ T-cell responses, respectively. Subsequently, IFNγ-producing CD8+ (B) or CD4+ (C) T cells were detected with intracellular flow cytometry staining. Indicated is the GMFI±SEM (n=4). ns: not significant, * p<0.05, ** p<0.01, *** p<0.005 and **** p<0.0001, ns: no significance

FIG. 11: Intravenous administration of poly (I:C)-containing liposomes strongly activates CD8⁺ T cells to produce interferon-y in an antigen-independent manner. Anionic liposomes [EPC: EPG: Cholesterol (3:1:2)] containing poly (I:C) were administered IV to mice (10 μg) alone or in combination with low or high doses of liposomes containing OTI/III model ovalbumin peptide (low dose is 22.5 nmol of phospholipid and 1,25 μg of peptide; high dose is 200 nmol of phospholipid and 11.1 μg of peptide) (n=4 per group except for naïve control which was n=3). On Day 7, H-2K^{b}/SIINFEKL tetramer binding T cells were identified (A). Splenocytes were restimulated ex vivo with SIINFEKL peptide for 5 h or 25 h, for CD8+ or CD4+ T-cell responses, respectively. Subsequently, IFNγ-producing CD8+ (B) or CD4+ (C) T cells were detected with intracellular flow cytometry staining. Indicated is the GMFI±SEM (n=4). ns: no significant, * p<0.05, ** p<0.01, *** p<0.005 and **** p<0.0001

FIG. 12: Effects of anionic phospholipid EPG relative molar amount concentration in poly (I:C) liposomes on the uptake and maturation by cDC1 cells. A single cell suspension of splenocytes was incubated with 0.5 μg of poly (I:C) encapsulated in liposomes. The EPG molar ratio in the liposomes was varied between samples (0, 4, 17, 33 and 67%), replacing EPC while cholesterol was kept constant (33% molar ratio). After incubation for 45 minutes at 37° C., splenocytes were extensively washed, and cells were incubated for 15 h at 37° C., after which the cells were stained. The uptake of liposomes (DiD) and expression of CD80 and CD86 by cDC1 cells were determined by flow cytometry analysis. Indicated is the geometric mean fluorescent intensity (GMFI) mean±SEM (n=3) for DiD (A), CD80 (B) and CD86 (C) by cDC1 cells.

FIG. 13: Uptake of anionic liposomes containing poly (I:C) by spleen cDC1 cells (A) and cDC2 cells (B) and red pulp macrophages (C). Mice were IV administered with 10 or 50 μg of soluble or anionic liposomal poly (I:C) [EPC: EPG: Cholesterol (3:1:2)]. After 24 hours, spleens were collected, and a single cell suspension was used for flow cytometry staining and analysis. Indicated are representative % of positive cells for DiD mean±SEM, as an indication of liposome of uptake by cDC1, cDC2 and red pulp macrophages ([n=4]. * p<0.05, ** p<0.01, *** p<0.005 and **** p<0.0001

FIG. 14: Enhanced therapeutic efficacy as a result of combined therapy of a-PD-L1 antibody and anionic liposomes containing poly (I:C). (A) Schematic experiment design of mice bearing B16F10 syngeneic tumors engrafted in the right flank after subcutaneous inoculation of 5×10⁵ B16F10 cells. After 7 days of inoculation, when tumor sizes were on average 100 mm³, mice were randomized into different groups and treatment was started. Mice were treated intravenously with free poly (I:C) (10 μg or 50 μg) or anionic liposomes containing poly (I:C) (10 μg) [EPC: EPG: Cholesterol (3:1:2)] or PBS (control) at days 0,3,6,9 and 12 after the start of treatment and treated intraperitoneally with a-PD-L1 (100 μg) or rat IgG antibody (100 μg) (control) at days 1,4,7,10 and 13 after the start of treatment. Indicated

(B) average tumor growth curves, (C) tumor growth inhibition of a-PD-L1 monotherapy and combinational poly (I:C) groups versus IgG control group and (C) normalized weight curves from start of treatment to day 5th, when the first mice reached a tumor size>1500 mm³ (humane endpoint HEP)). (D) Survival curves of treated mice that reach tumor size humane endpoint (HEP) (tumor size>1500 mm³) over time from start of treatment to one day after the end of treatment (day 14th). Animals that reached another HEP (e.g. tumor ulceration) but not tumor size HEP were not considered in the survival analysis. Black arrows indicated poly (I:C) and grey arrows indicated a-PD-L1 injection. Indicated is the Mean±SEM (n=10, except IgG group which n=8 and a-PD-L1/poly (I:C) 10 ug which n=9). * p<0.05, ** p<0.01, *** p<0.005.

FIG. 15: Enhanced therapeutic efficacy by combining a-PD-L1 antibody with anionic liposomes containing poly (I:C). Individual tumor volumes (length×with²)/2 over time after start of treatment. Indicated is the Mean±SEM (n=10, except IgG group which n=8 and α-PD-L1/poly (I:C) 10 ug which n=9). * p<0.05, ** p<0.01, *** p<0.005.

FIG. 16: Effect of the lipid composition on the characteristics of anionic liposomes containing RNA.

RNA-anionic liposome formulations with different molar ratios of EPC, EPG and Cholesterol were prepared in a design mixture experiment (n=17) (F). Particle mean size (A), PDI (B), zeta potential (C), RNA encapsulation efficiency (D) and TLR3 activation (%) vs free poly (I:C) by HEK 293T TLR3⁺ cells (E) were analysed for each formulation. RSMs were built for each parameter. Indicated is mean±SEM of analytical replicates (n=3).

FIG. 17: Effect of design A microfluidics process parameters on the particle characteristics of anionic liposomes containing poly (I:C).

RNA-anionic liposomes formulations were prepared with different aqueous RNA phase flow rate, ethanolic lipid phase flow rate, lipid concentration and RNA concentration in a response surface designed experiment (n=30) (D). Particle mean size (A), PDI (B) and RNA encapsulation efficiency (C) were analysed for each formulation. RSMs were built for each parameter. Indicated is mean±SEM of analytical replicates (n=3). N.D.=non detectable. N.M.=not measurable.

FIG. 18: Effect of design B microfluidics process parameters on the particle characteristics of anionic liposomes containing poly (I:C).

RNA-anionic liposomes formulations were prepared with different aqueous RNA phase flow rate, ethanolic lipid phase flow rate, water phase flow rate, lipid concentration and RNA concentration in a response surface designed experiment (n=53) (D). Particle mean size (A), PDI (B) and RNA encapsulation efficiency (C) were analysed for each formulation. RSMs were built for each parameter. Indicated is mean±SEM of analytical replicates (n=3).

FIG. 19: Increasing poly (I:C) (pIC)/lipid weight ratio decreases the scattering intensity of anionic liposomes (AL) in 100% D₂O (A) and increases the scattering neutron density in the shell (B). The scattering patterns of pIC encapsulated in nanoparticles and pIC externally added to preformed empty nanoparticles are different (C). When the pIC is externally added to and incubated with preformed empty anionic liposomes, there is an increase of scattering at low Q (0.1 to 1 Å⁻¹) which indicates an increase in the background. The increase in background can be attributed to more hydrogen in the D₂O solvent following the addition of pIC, suggesting that pIC is localized in the solvent rather than in the nanoparticles. No appreciable increase in the background is observed in case of nanoparticles with encapsulated pIC, suggesting that pIC is localized in the nanoparticles rather than in the solvent. Comparison of the scattering patterns of anionic liposomes (AL) and LNP before (empty nanoparticles) and after the encapsulation of pIC (D). The encapsulation of pIC in LNP but not in anionic liposomes modifies the scattering profile, suggesting a rearrangement of the internal LNP structure. The rearrangement observed in LNP may be explained by the electrostatic interactions between the ionizable cationic lipid and the anionic pIC, which do not occur in the pIC-AL. Additionally, pIC-AL have a distinctive structure compared to pIC-LNP. Symbols represent SANS data and solid lines correspond to the best fit using the sphere core-shell model (A), (B) and (D).

FIG. 20: The strength of the interaction between RNA incubated with preformed empty DMPC vesicles and RNA encapsulated in DMPC vesicles was studied using asymmetrical flow field-flow fractionation (AF4). Encapsulation of RNA enables a strong interaction of RNA with DMPC vesicles while the interaction of RNA incubated with DMPC vesicles is weak and reversible. DMPC vesicles and/or poly (I:C) (pIC) were incubated with RNAse free water. Later, Ribogreen dye was supplemented for the fluorescence detection of RNA and samples were analyzed by AF4. RNA was determined using a fluorescence detector (λₑₘ=495 nm, λₑₓ=520 nm) (left axis) and the light scattering intensity of the particles were determined using MALS (right axis). Indicated are (A) soluble pIC, (B) preformed empty DMPC vesicles, (C) preformed empty DMPC vesicles incubated with externally added RNA and (D) DMPC vesicles with encapsulated RNA. Melting temperatures of DMPC vesicles were obtained by differential scanning calorimetry measurements, showing a significant decrease of the melting temperature only when RNA is encapsulated in the DMPC vesicles, suggesting an interaction of RNA within the hydrophobic region of DMPC vesicles. n=3 analytical replicates.

FIG. 21: Anionic liposomes containing poly (I:C) (pIC-AL) have strong anti-tumoral effects when administered intravenously both as monotherapy and in combination with an a-PD-L1 antibody in a colon carcinoma model. Mice were engrafted with mice colon carcinoma syngeneic tumors after subcutaneous inoculation of 5×10⁵ MC38 cells in the right flank. After 7 days of inoculation, when tumor sizes were on average ~ 100 mm³, mice were randomized into different groups and treatment was started (day 0). Mice were treated intravenously with pIC-AL (10 μg) or PBS (control) at days 0,3,6,9 and 12 after the start of treatment and treated intraperitoneally with a-PD-L1 (100 μg) or rat IgG antibody (100 μg) (control) at days 1,4,7,10 and 13 after the start of treatment. (A) Average tumor growth curves from start of treatment (day 0) to day 7, when the first mice reached a tumor size>1500 mm³ (which is the humane endpoint (HEP). Indicated is the mean±SEM (n=8). (B) Survival curves of treated mice over time from day 0 to day 60 after start of treatment. (C) Cured mice (n=3) and naïve control mice (n=3) were rechallenge with a subcutaneous inoculation of 5 ×10⁵ MC38 cells in the left flank at day 60 after start of treatment. Average tumor growth curves from the rechallenge with MC38 cells until all control mice reached the tumor size HEP. Indicated is the mean±SEM. (D) Characterization of the immune cells in the colon carcinoma tumor microenvironment after the treatment with pIC-AL. Mice were engrafted with mice colon carcinoma syngeneic tumors after the inoculation of 5×10⁵ MC38-CEA in subcutaneous mammary fat pad. After 6 days of inoculation, when tumor sizes were on average ~ 100 mm³, mice were randomized into different groups and treatment was started. Mice were treated intravenously with pIC-AL (10 μg) or PBS (control) at days 0 and 3 after the start of treatment. After 5 days of start of treatment, tumors were collected, and a single cell suspension was used for flow cytometry staining and analysis of tumor infiltrating lymphocytes (TILs) and myeloid suppressing derived cells (MSDCs). ns: not significant, *p<0.05, ** p<0.01, *** p<0.005 and **** p<0.0001

FIG. 22: Anionic liposomes containing poly (I:C) (pIC-AL) has strong anti-tumoral effects as monotherapy in an orthotopic hepatic liver cancer model. Hepatocellular carcinoma tumor cells (Hepa 1-6:5×10⁶ cells/mouse) in 100 μl of PBS were injected via the mesenteric vein into the mouse liver. Tumor growth was monitored weekly by MRI imaging. Mice (n=5) that had detectable tumors were divided in two groups: 1) Control (n=2) and pIC-AL (10 μg) (n=3). Treatment (PBS or pIC-AL) was administered two times per week for 4 weeks or until reaching the tumor size HEP. Tumor growth was followed up until reaching the humane end point up to 8 weeks after start of treatment. Indicated are representative MRI images of PBS treated mice at one day before start of treatment (A) and day 13 after start of treatment (B). C-E indicate representative MRI images of treated mice with pIC-AL one day before start of treatment (C), day 13 (D) and day 27 (E) after start of treatment.

FIG. 23: Anionic liposomes containing poly (I:C) (pIC-AL) induce a strong anti-tumoral activity without the acute toxicity associated with pIC-LNP: Anti-tumoral activity, acute toxicity, and cytokine analysis after intravenous administration of pIC-AL or pIC-LNP administered both as monotherapy and in combination with an a-PD-L1 antibody. Mice were engrafted with mice colon carcinoma syngeneic tumors after subcutaneous inoculation of 5×10⁵ MC38 cells in the right flank. After 7 days of inoculation, when tumor sizes were on average ~ 100 mm³, mice were randomized into different groups and treatment was started. Mice were treated intravenously with pIC-AL (10 μg), pIC-LNP (10 μg) or PBS (control) at day 0 after the start of treatment and treated intraperitoneally with a-PD-L1 (100 μg) or PBS (control) at day 1 after the start of treatment. Indicated are (A) lethality (%) and (B) weight loss after 3 days of start of treatment, (C) average tumor growth curves from day 0 to day 3 after start of the treatment. Indicated is the mean±SEM (n=6) for all groups expect for pIC-AL (n=4)). Intravenous injection of pIC-LNP but not pIC-AL induces dramatically strong pro-inflammatory cytokine responses. Indicated are the plasma concentration (left Y axis) and fold change (or fold enhancement) versus the PBS group (right Y axis) for the following pro-inflammatory cytokines: IL-6 (D), INF-α (E), INF-β (F). INF-γ (G), TNF-α (H) and IL-1β (I)) after 3 hours of the administration of pIC-AL or pIC-LNP nanoparticles or PBS (n=6 for PBS group, n=10 for pIC-AL and n=12 for pIC-LNP). No a-PD-L1 was administered at the blood sampling time of 3 hours after start of treatment. ns: not significant, *p<0.05, ** p<0.01, *** p <0.005 and **** p<0.0001

FIG. 24: Histopathology and plasma biochemistry analysis show strong liver toxicity in mice in case of treatment with LNP containing poly (I:C) (pIC-LNP) but not in case of treatment with anionic liposomes containing poly (I:C) (pIC-AL). Mice with MC38 tumors engrafted (continuation of FIG. 23) were treated intravenously with pIC-AL (10 μg), pIC-LNP (10 μg) or PBS (control) at the start of treatment (day 0) and additionally treated intraperitoneally with a-PD-L1 (100 μg) or PBS (control) at day 1 after the start of treatment. 3 days after start of treatment, surviving mice were euthanized and vital organs and blood samples were collected. Representative haematoxylin and eosin (H&E) staining of liver slides 3 days after start of treatment are shown; magnification 20×. Indicated are (A) Control, (B) pIC-AL, (C) pIC-AL plus a-PD-L1 antibody, (D) pIC-LNP and (E) pIC-LNP plus a-PD-L1 antibody, showing strong liver damage in the pIC-LNP group which is further worsened when a-PD-L1 antibody is administered 1 day later. No signs of liver toxicity were observed in case of mice treated with pIC-AL monotherapy or pIC-AL plus a-PD-L1 antibody. (F) Mean enzymatic activity (UI/L)±SEM of the hepatic enzyme AST (F). Elevation of AST activity was only observed in mice treated with pIC-LNP (Only surviving mice were considered for acute liver toxicity analysis: n=6 for PBS group, n=5 for pIC-LNP, n=2 for pIC-LNP plus a-PD-L1 groups, n=4 for pIC-AL and n=6 for pIC-AL plus a-PD-L1 group). ns: not significant, *p<0.05, ** p<0.01, *** p<0.005 and **** p<0.0001

FIG. 25: Anionic liposomes containing non-immunogenic mRNA are not inflammatory and do not induce the activation of the immune system. Mice were receiving IV 10 μg of non-immunogenic mRNA encapsulated in anionic liposomes (mRNA-AL) or in LNP (mRNA-LNP). Blood samples were taken 3 hours after IV injection for the measurement of cytokines. After 24 hours, mice were sacrificed, the spleen was collected, and a single cell suspension of the spleen was used for flow cytometry staining and analysis. Indicated are representative geometric mean fluorescence intensity (GMFI)±SEM of the CD80 maturation marker expressed by cDC1 (A) and by cDC2 (B) and of the activation marker CD25 expressed by T cells, respectively (n=2). Indicated are the serum inflammatory cytokine concentration IL-6 (D), INF-α (E), INF-β (F). INF-γ (G), TNF-α (H) and IL-1β (I) as mean±SEM (left Y axis) and fold enhancement mean±SEM (right Y axis) after 3 hours of the IV injection (n=4). ns: not significant, *p<0.05, ** p<0.01, *** p<0.005 and **** p<0.0001

The following Examples illustrate the different embodiments of the invention.

### EXPERIMENTAL SECTION

### Materials and Methods

### Preparation of Liposomes

Liposomes containing poly (I:C) were prepared using a single or double microfluidics micro-mixer, which enables the nanoassembly of lipids through a hydrodynamically focused flow. A mixture of egg phosphatidylcholine (EPC), egg phosphatidylglycerol (EPG) (Lipoid GmbH, Ludwigshafen, Germany) and cholesterol (Sigma Aldrich, Darmstadt, Germany) were dissolved in ethanol. Lipid concentration and the molar ratio were studied during the design of experiments, resulting in a final 5 mM lipid concentration and 3:1:2 (EPC: EPG: Cholesterol) molar ratio for in vivo experiments. Where indicated, the ethanol phase was supplemented with GM3 (3 mol %) (Avanti Polar, Alabaster, AL, US) and/or 0.1 mol % of the lipophilic fluorescent tracer DiD (1′-dioctadecyl-3,3,3′,3′-tetramethyl indodicarbocyanine, Life Technologies, Frederick, MD, USA). Poly (I:C) (high molecular weight (HMW), InvivoGen, San Diego, US) was dissolved in RNase-free water (Qiagen, Hilden, Germany). Lipids and poly (I:C) were mixed using the Dolomite microfluidics system (Royston, UK). Two system variants were explored: In Design A, one large droplet junction chip for laminar flow (Part number 3200130) or a micromixer chip for chaotic mixing (Part number 3200401) was employed and held in an interface H (Part No. 3000155). In design B, two large droplet junction chips (Part number 3200130) were used and held and connected by a double H interface (Part system 3200088). In Design A, the two fluids (lipids dissolved in ethanol and RNA dissolved in water) were delivered into the chip inlets with two pressure pumps using FEP tubing of OD 1.6 mm and a linear connector 4-way (Part number 3000024). In Design A, the two fluids (lipids dissolved in ethanol and RNA dissolved in water) were delivered into the chip inlets with two pressure pumps using FEP tubing of OD 1.6 mm and a linear connector 4-way (Part number 3000024). In design B, the three fluids (lipids dissolved in ethanol, RNA dissolved in water and additional water flow in the latest chip) were delivered into the chip inlets with three pressure pumps using FEP tubing of OD 1.6 mm and a linear connector 4-way (Part number 3000024). Total flow rates were controlled using two (Design A) or three (Design B) Mitos Flow Rate sensors (Part numbers 32000097 and 3200096). After microfluidics mixing, the samples were recollected and further diluted with RNase-free water to reach an ethanol concentration lower than 10%. Subsequently, samples were concentrated using Vivaspin 20 centrifugal concentrators (300 kDa MWCO) (Sartorius Stedim Biotech GmbH, Göttingen, Germany) at a spin of 1000 g. Once concentrated (approximately 200 μL), the samples were diluted to 20 mL using RNase-free water and further concentrated using Vivaspin 20 centrifugal concentrators (300 kDa MWCO). Finally, samples were resuspended in sterile PBS (Sigma Aldrich, Darmstadt, Germany).

For SANS experiments, synthetic anionic liposomes were prepared using DPPC, POPC, DPPG, POPG (Lipoid) and Cholesterol (Sigma Aldrich) as there is a commercially available deuterated version of these lipids. DPPC and POPC are the major saturated and unsaturated lipid components of egg PC while DPPG and POPG are the major saturated and unsaturated lipid components of egg PG. In some samples, undeuterated lipids were replaced with their deuterated versions: DPPC-d62 (860355P), POPC-d31 (860399P), DPPG-d62 (860355P), POPG-d31 (860384P), Cholesterol-d7 (700041P) (Avanti Polar). Lipids were dissolved in ethanol in a 5 mM lipid concentration and a 3:3:1:1:4 molar ratio (DPPC: POPC: DPPG: POPG: Cholesterol) and mixed with a 500 μg/mL solution of Poly (I:C) and later D₂O in a flow ratio 1:3:9 using the Design B microfluidic set-up described before.

For SANS experiment involving anionic liposomes ((DPPC: POPC: DPPG: POPG: Cholesterol) with increasing RNA: lipid weight ratio (%), the preparation were conducted as follows: For 0% and 5% weight ratio, the formulation were prepared as previously described; for 0% weight ratio, a 5 mM lipid ethanol solution was mixed with a 500 μg/mL poly (I:C) solution, followed by the addition of D₂O in a flow ratio of 2:3:9; for 15% weight ratio, a 5 mM lipid ethanol solution was mixed with a 500 μg/mL poly (I:C) solution, and D₂O was subsequently added in a flow ratio of 1:1:3; for 30% weight ratio, a 5 mM lipid ethanol solution was mixed with a 1000 μg/mL poly (I:C) solution, and D₂O was added in a flow ratio of 1:1:3 using the Design B microfluidic set-up described before.

For DMPC DSC and AF4 experiments, DMPC was dissolved in ethanol in a 5 mM lipid concentration and mixed with a 500 μg/mL solution of Poly (I:C) and later water in a flow ratio 3:3:9 using the Design B microfluidic set-up described before.

For SANS experiments and in vivo experiments, lipid nanoparticles (LNP) containing poly (I:C) or Fluc mRNA (Ribopro, The Netherlands) were prepared D-lin-MCR3-DMA (Medchem), Cholesterol (Sigma), DSPC (Lipoid) and DMG-PEG2000 (Avanti Polar) with a molar ratio of 50:38.5:10:1.5. Lipids were dissolved in ethanol in a 5 mM lipid concentration and mixed with a 500 μg/mL solution of Poly (I:C) and later D₂O (SANS) or water in a flow ratio 1:3:9 using the Design B microfluidic set-up described before.

### Liposome Characterization

The phosphate content of the liposomal preparations was determined through an acidic digestion according to Rouser et al. (Lipids 5, 494-496 (1970)). Liposome sample dilutions and known amounts of a 0.5 mM KH₂PO₄ standard solution were transferred into clean glass tubes, and the solvent was completely evaporated using a heat block at 220° C. Subsequently, 0.3 mL of perchloric acid was added to each tube and placed in the heat block for 60 min or until the yellow color disappeared. When cooled down, 1 mL of water, 0.5 mL of molybdate solution and, subsequently, 0.5 mL of ascorbic acid solution were added and agitated on a vortex. The tubes were placed in a warm water bath for 5 min and then cooled down. Next, the absorbance of the samples and standard was measured at 797 nm, using a spectrophotometric microplate reader (BMG SPECTROstar Nano, De Meern, the Netherlands).

The hydrodynamic diameter and polydispersity index (PDI) were measured by dynamic light scattering (DLS) using a Malvern Zetasizer Nano S (Malvern Instruments, Malvern, UK) equipped with a He—Ne 633 nm laser configured with a scattering angle of 173°. The measurements were performed using Malvern's Zetasizer v7.13 software (Malvern Instruments, Malvern, UK). A minimum of 3 measurements were performed per sample, and the measurement duration was automatically set by the software depending on the samples' characteristics. A viscosity of 0.8872 centipoises (cP) and refractive index (RI) of 1.330 for the dispersant and an RI of 1.590 and absorption of 0.010 for the material in suspension were set on the software. The samples were diluted in PBS, and measured at 25° C. A PDI<0.3 was considered acceptable, indicating a homogenous size distribution of sufficient homogeneity.

The zeta potential was determined using Zetasizer Nano Z (Malvern Instruments, Malvern, UK) and Malvern's Zetasizer v7.13 software. A minimum of 3 measurements were performed per sample, and the measurement duration was automatically set by the software depending on the samples' characteristics. The samples were diluted into 0.1× PBS and measured at 25° C. A viscosity of 0.8872 cP, RI of 1.330 and dielectric constant of 78.5 for the dispersant and an RI of 1.590 and absorption of 0.010 for the material in suspension were set on the software.

The poly (I:C) content was determined using RiboGreen RNA quantification reagent (Jones et al. Anal Biochem 265, 368-374 (1998)) (Thermofisher). Quantification of poly (I:C) in the liposomal formulation was conducted using a standard curve generated from a dilution series of the corresponding poly (I:C) stock. Both samples and standards were diluted in Triton X-100 0.5% (Sigma) solution in RNase-free water (Qiagen). Samples were diluted to reach a theoretical concentration of 1 μg/mL. Samples were placed in a black polystyrene 96 wells plate and diluted 1:1 with RiboGreen reagent (previously diluted 1/200 in RNase-free water). Fluorescence was measured using a FP-8300 spectrofluorometer (Jasco) set at λ_{excitation}=485 nm, λₑₘᵢₛₛᵢₒₙ=520 nm. Sample concentration was obtained through a standard curve that was calculated by linear regression analysis of the fluorescence intensity plotted against the concentration of the standard poly (I:C). Encapsulation efficiency was calculated by actual poly (I:C) concentration divided by theoretical poly (I:C) concentration (total poly (I:C) added divided by final formulation volume) as follows:

$$Encapsulation efficiency ( \% ) = ( \frac{Actual RNA concentration}{\frac{Intial RNA mass}{F inal formulation volume}} ) \times 100 \%$$

TLR3 activation and liposome uptake in HEK-Blue™-hTLR3⁺ cells.

HEK-Blue™-hTLR3 reporter cell line (Invitrogen) is stability transfected with TLR3 gene and an inducible SEAP (secreted embryonic alkaline phosphatase) reporter gene coupled to the NF-κB/AP-1 promoter. The HEK-Blue™-hTLR3 reporter cell line was cultured in Dulbecco's Modified Eagle's Medium (DMEM) (Sigma) with high glucose (4.5 g/L), L-glutamine (2 mM), fetal bovine serum (FBS). To avoid unspecific TLR3 activation due to the presence of RNA in the medium supplement with FBS, cells were plated in DMEM without FBS in a 96-well plate (40.000 cells in 100 μL per well). After 24 hours, 22 ng of free or encapsulated poly (I:C) resuspended in 20 μL PBS was added to the well. PBS was used as negative control. Additionally, 100 μL of QUANTI-Blue™ Solution (Invitrogen, San Diego, US) was added to quantify SEAP. (Final poly (I:C) concentration=0.1 μg/mL). After 12 hours, SEAP activity (absorbance), representing activation of TLR3/NF-κB/AP-1TLR3 was measured at 650 nm on a Bio-Rad microplate reader (Hercules, California, US). TLR3 activation was compared to free poly (I:C) with the following equation:

$$TLR 3 activation ( \% ) = \frac{( Sample {abs}_{650 nm} - Negative control {abs}_{650 n m}}{Free poly ( I : C ) {abs}_{650 nm} - Negative control {abs}_{650 n m}}$$

For liposome uptake experiment, cells were plated in DMEM without FBS in a 96-well plate (40.000 cells in 100 μL per well). After 24 hours, 22 ng of free or encapsulated poly (I:C) resuspended in 20 μL PBS was added to the well. Empty anionic liposomes (EPC: EPG: cholesterol 3:1:2) and PBS were used as controls. After 4 hours, the cell media was removed, cells were cleaned with 1% FBS in PBS, incubated with Zombie NIR viability dye (Biolegend, US) and later fixated with 1% PFA. Afterwards, DiD geometric mean fluorescent intensity (GMFI) was measured by BD FACSCanto flow cytometer (BD, East Rutherford, NJ, US) and data analysed with Flowlogic software (Inivai, Mentone, Australia).

Molecular weight analysis of encapsulated RNA and plasma stability of anionic liposomes containing RNA. Separation of encapsulated RNA and adsorbed RNA on DMPC vesicles employing AF4.

The molecular weight of encapsulated RNA and the plasma stability of anionic liposomes containing RNA was analysed using an AF2000 Asymmetrical Flow Field-Flow Fractionation system (Postnova, Landsberg am Lech, Germany). This system consists of a solvent degasser (Postnova, PN7520), Focus pump and Tip pump (Postnova, PN1130), Cross-flow pump (Postnova, AF2000, AF2000-MF), autosampler (Postnova, PN5300), an asymmetric AF4 channel (Postnova, PN4020), multi-angle light scattering detector (MALS) (Postnova, PN3621), RI detector (Postnova, PN3150), dual wavelength absorbance detector (Waters, 2487), multi-wavelength fluorescence detector (Waters, 2475) and a Zetasizer Nano ZS detector (Malvern).

Encapsulated poly (I:C) was extracted from anionic liposomes by precipitation with isopropanol 100%. The clean pellet of RNA was resuspended in PBS and injected in a size chromatography (SEC) column (TSKgel® G-DNA-PW) running in PBS (flow rate=0.5 mL/min, 25° C.). Additionally, free poly (I:C) and standard DNA ruler (SM1331, Thermo scientific, US) samples were treated similarly and injected into the SEC column. A UV detector at 2=260 nm was used for RNA detection and a multi-angle light scattering (MALS) detector for molecular weight determination using NovaFFF Suite software (Postnova).

The stability of empty and RNA-containing anionic liposomes in human plasma was studied using asymmetrical flow field-flow fractionation (AF4). Liposomes (2 mM of lipids) were incubated with PBS or 20% human plasma (Sera Laboratories, UK) at 37° C. for 2 hours and later analysed in AF4. The radius of gyration (Rg) and light scattering intensity of the particles were determined using MALS and protein corona formation was determined using a fluorescence detector (λem=280 nm, λem=340 nm). In the AF4 channel, 350 μm spacer and regenerated cellulose membrane with 10 kDa molecular weight cut-off (Z-AF4-MEM-612-10 kDa, Postnova) was used. Samples were fractionated using a multi-step linear and power-field decay program. The detector flow was maintained at 0.5 mL/min. PBS was filtered using a 0.1 μm filter prior to use.

The strength of the interaction between RNA incubated with preformed empty DMPC vesicles and RNA encapsulated in DMPC vesicles was studied using AF2000 Asymmetrical Flow Field-Flow Fractionation system (Postnova, Landsberg am Lech, Germany) described before. DMPC vesicles and/or poly (I:C) were incubated in RNAse free water. Later, Ribogreen dye was supplemented for the detection of RNA and samples were analyzed by AF4. RNA was determined using a fluorescence detector (λem=495 nm, λex=520 nm) and the light scattering intensity of the particles were determined using MALS. In the AF4 channel, 350 μm spacer and regenerated cellulose membrane with 10 kDa molecular weight cut-off (Z-AF4-MEM-612-10 kDa, Postnova) was used. Samples were fractionated using a multi-step linear and power-field decay program. The detector flow was maintained at 0.5 mL/min. Purified water was filtered using a 0.1 μm filter prior to use. In this experiment, purified water was used instead of PBS for avoiding the aggregation of DMPC vesicles.

### RNase Protection Assay

The protection that anionic liposomes confer against nuclease degradation was evaluated using RNase III, which converts long double-stranded RNA into a heterogeneous mix of short (18-25 bp) RNAs. 10 μg of free poly (I:C) and encapsulated poly (I:C) were incubated with ShortCut® RNase III (M0245, New England Biolabs, US) according to the manufacturer's instructions for 2 h at 37° C. in reaction buffer or in reaction buffer supplemented with human plasma (20% v/v), then samples were separated using agarose gel (1%) electrophoresis and visualized with 5 μL Midori Green (Nippon Genetics, Germany) staining per 100 mL of agarose. Additionally, 5 μL of standard DNA ruler (SM1331, Thermo scientific, US) was added in the first well of the agarose gel.

### Transmission Electron Microscopy:

The size and shape of empty and RNA-containing anionic liposomes particles were analyzed by taking transmission electron microscopy (TEM) photographs using a Tecnai 12 TEM microscope (FEI Company, The Netherlands), operating at 80 kV. 10 μL of diluted particle suspension (2 mM phospholipids) were placed in a previous glow discharged Formvar/Carbon coated copper grid and contrasted with uranyl oxalate (pH 7), and then contrasted-embedded in a mixture of 2% methyl cellulose/4% uranyl acetate (pH 4).

### Small-Angle Neutron Scattering Measurements

Small-angle neutron scattering (SANS) measurements were performed at Larmor beamline of the ISIS pulsed neutron source at the Rutherford Appleton Laboratory (Didcot, UK). Two type of anionic liposomes were prepared: Concentrated suspension of anionic liposomes were prepared as described previously but replacing RNase free water with deuterium oxide (D₂O) (158122, Sigma Aldrich). Empty and RNA containing anionic liposomes (4 mg/mL concentration) were loaded in 1 mm path length, 1 cm width, quartz Hellma cells, which were placed in a temperature-controlled sample holder kept at 20° C. Concentrated suspension of anionic liposomes were prepared as described previously but replacing RNase free water with deuterium oxide (D2O) (158122, Sigma Aldrich). Before measurement, samples were resuspended into 100/0, 68/32, 50/50, and 27/63 D2O/H2O (%/%) water. Time of flight scattering measurements were collected for 30 min per sample. The wavelength and Q range for this experiment were 0.9-12.5 Å and 0.004-0.8 Å⁻¹ respectively. Data were normalised to sample transmission and corrected for detector efficiencies and the scattering from an empty cell. Data reduction was performed using Mantid and scattering simulations fitted using SasView v5.0.2 (www.sasview.org). All measurements were fitted using a sphere, core-shell model.

### DSC Measurements

DSC measurements were conducted on a TA Instruments Discovery DSC (TA Instruments, Etten-Leur, The Netherlands). For poly (I:C) melting temperature analysis, samples were placed in DSC hermetic aluminum pans and the samples were conditioned at 10° C. for 10 min, next, the samples were heated at 0.5° C./min to 100° C. and subsequently cooled. For DMPC vesicles melting temperature analysis, samples were placed in DSC hermetic aluminum pans. 3 heating cycles (1° C./min) were performed starting at a temperature of 10° C. and with the final temperatures of 50° C., 90° C. and 50° C., respectively, for each cycle. Data was analyzed using Trios v3.3.0.4055 software.

### Measurement of Membrane Fluidity by GP Laurdan Measurement

Empty anionic liposomes, anionic liposomes containing RNA or empty liposomes plus free RNA (50 μM lipids, 2 μg/mL RNA) were incubated with 2,5 UM laurdan in RNase-free water. Liposomes suspension was transferred to quartz cuvettes and measured in FP-8300 spectrofluorometer (Jasco) equipped with a temperature-controlled sample holder. A temperature ramp was set from 10° C. to 80° C. The temperature was set to 24° C. or 37° C. Samples were excited at 356 nm and the emission intensity was recorded at 440 nm and 490 nm. The general polarization (GP) of laurdan was calculated as,

$$G P = \frac{{I}_{440} - {I}_{490}}{{I}_{440} + {I}_{490}} \qquad ( Equation 4 )$$

### Animal Experiments:

C57BL/6 WT mice were bought from Charles River or bred in-house. Male and female mice between 8 and 24 weeks of age were used for maturation studies. Female mice between 8 and 12 weeks were used for T cell priming experiments. Female mice between 8 and 10 weeks were used for tumor animal experiment.

### Enzymatic Spleen Digestion

Spleens from C57BL/6 wild-type (WT) were mechanistically dissociated and subsequently digested using a mixture of 3 mg/mL lidocaine, 2 Wünsch units/mL Liberase TL (Roche, Mannheim, Germany) and 50 mg/mL DNase I (Roche) for 12 min at 37° C. with continuous stirring [38]. Subsequently, cold medium (RPMI-1640 (Gibco, Life Technologies) supplemented with 10% heat-inactivated FCS (Biowest), 10 mM ethylenediaminetetraacetic acid (EDTA), 20 mM HEPES and 50 μM 2-mercaptoethanol) was added, and digestion was continued for an additional 10 min at 4° C. with continuous stirring. After digestion, red blood cells were lysed using ammonium-chloride-potassium lysis buffer, and the splenocytes were filtered through a 70-100 μm filter.

### In Vitro Liposome Uptake and Dendritic Cell Maturation

Enzymatically digested splenocytes were plated and washed once. Free or encapsulated Poly (I:C) in liposomes were diluted in PBS, added to 3 million cells in the concentrations indicated in the figures and incubated for 45 min at 37° C. The DiD signal in various immune cell populations was assessed after flow cytometry staining, as described below.

### In Vivo Liposome Uptake, Dendritic Cell Maturation, Serum Cytokines and Chemokines and Hepatic Enzymes Quantification

Mice were IV injected with free or encapsulated poly (I:C) in anionic liposomes (as indicated in the figures). Blood samples were taken after 3 hours of IV injection for cytokine and chemokines measurements. After 24 hours, mice were sacrificed, and blood was collected, allowing it to clot. The clot was removed by centrifugation, and the serum was used to quantify serum anti-viral cytokines and chemokines, and hepatic aspartate transaminase (AST) and alanine transaminase (ALT), as described below. Additionally, spleens were collected, and a single cell suspension was used for flow cytometry staining and analysis, as described below.

### Anti-Viral Cytokine and Chemokine Quantification

Cytokine and chemokine secretion was analyzed using the LEGENDplex™ mouse anti-virus response panel (13-plex) multiplex assay (740621, BioLegend®) according to the manufacturer's protocol in combination with the flow cytometer FACSCanto™ ∥ (BD).

### Serum ALT and AST Activity Assay

ALT activity was determined by using the alanine aminotransferase kit (MAK052, Sigma Aldrich) according to the manufacturer's instructions in combination with a FP-8300 spectrofluorometer (Jasco).

AST activity was determined by using the aspartate aminotransferase kit (MAK055), Sigma Aldrich) according to the manufacturer's instructions and with a spectrophotometric microplate reader (BMG SPECTROstar Nano or Spectramax ID3 (Molecular Devices)).

### In Vivo T-Cell Priming

Mice were IV injected with free or encapsulated poly (I:C) in liposomes (as indicated in the figures). On Day 7, the mice were sacrificed and the spleens were isolated. Where indicated, blood was collected via a cardiac puncture. The spleens were mashed through a 70-100 μm filter to obtain a single-cell suspension. Red blood cells were lysed using ammonium-chloride-potassium lysis buffer. Subsequently, the cells were used for peptide restimulation and flow cytometry analysis.

### Flow Cytometry and Antibodies for T Cell Priming Experiments

Splenocytes were incubated with 10 μg/mL of anti-CD16/32 (clone 2.4G2, in-house produced) for 15 min at 4° C. to block unspecific Fc receptor binding and stained for 30 min using the following antibodies: anti-CD169 (clone SER-4, in-house produced), anti-B220 (clone RA3-6B2, BD Biosciences), anti-F4/80 (clone T45-2342, BD Biosciences), anti-CD8a (clone 53-6.7, BD Biosciences), anti-CD11c (clone HL3, BD Biosciences), anti-I-A/I-E (clone M5/114. 15.2, BD Biosciences) and the Fixable Viability Dye eFluor 780 (eBioscience, San Diego, CA, USA) diluted in PBS/0.5% bovine serum albumin (BSA).

Alternatively, splenocytes were stained with anti-XCR1 (clone ZET, BioLegend, San Diego, CA, USA), anti-CD11c (clone HL3, BD Biosciences), anti-I-A/I-E (clone M5/114.15.2, BioLegend), anti-Ly6G (clone 1A8, BioLegend), anti-BST2 (clone 129C1, BioLegend), anti-CD11b (clone M1/70, BioLegend), anti-CD169 (clone SER-4, in-house produced), anti-Siglec-H (clone 551, BioLegend), anti-F4/80 (clone T45-2342, BD Biosciences), anti-CD8a (clone 53-6.7, BD Biosciences), anti-Sirpa (clone P84, BioLegend); the lineage markers anti-CD3e (clone 145-2C11, BioLegend), anti-CD19 (clone 6D5, BioLegend) and anti-NK1.1 (clone PK136, BioLegend); and the Fixable Viability Dye eFluor 780 (eBioscience) diluted in PBS/0.5% BSA.

OVA-specific CD8+ T cells were identified by staining with anti-CD8a antibody (clone 53-5.6, BD Biosciences), anti-CD44 antibody (clone KM81, ImmunoTools, Friesoythe, Germany) and PE-labelled H-2K^{b}/SIINFEKL tetramer (LUMC, Leiden, The Netherlands) at 37° C. for 60 min. Splenocytes were restimulated for 5 h with OVA257-264 in the presence of Golgiplug (BD Bioscience) or 20 h with OVA262-276, followed by 5 h of incubation with Golgiplug. Intracellular IFNγ was detected by staining with an anti-CD11a antibody (clone M17/4, eBioscience) and anti-CD8 antibody (clone 53-5.6, BD Biosciences), or anti-CD4 antibody (clone GK1.5, eBioscience). After surface staining, the cells were fixed using 2% PFA (Electron Microscopy Sciences, Hatfield, PA, USA). For intracellular IFNγ detection, the cells were permeabilized using 0.5% saponin buffer and stained with anti-IFNγ antibody (clone XMG1.2, eBioscience) for 30 min at 4° C.

### Tumor-Bearing Animal Experiments

### B!6F 10 Model

The B16F10 melanoma cell line was kindly gifted by Dr Ingrid Molema (University Medical Center Groningen (UMCG), The Netherlands). B16F10 cells were grown in high-glucose Dulbecco's Modified Eagle's Medium (DMEM; Sigma Aldrich) supplemented with 10% fetal bovine serum (FBS; Sigma Aldrich) and cells were split when they reached 85% confluence.

5×10⁵ B16F10 tumor cells (in 100 μL DMEM without FBS) were first injected subcutaneously into the right flank of C57BL/6. Tumors were detected after 5-7 days after implementation. Tumor size was monitored daily using a digital caliper and the following formula: Volume (mm³)=(length×with²)/2. After 7 days of tumor implementation (day 0 of start of treatment), the tumors reached an average tumor volume of 100 mm³ and 57 mice were randomized into 6 treatment groups: PBS/IgG (n=8), PBS/α-PD-11 (n=10), free poly (I:C) (10 μg)/a-PD-L1 (n=9), free poly (I:C) (50 μg)/a-PD-L1 (n=10), poly (I:C) encapsulated in anionic liposomes (10 ug)/a-PD-L1 (n=10). PBS or free or encapsulated poly (I:C) were administered intravenously on days 0,3,6,9,12 after the start of treatment. 100 μg of Rat IgG2b isotype control (clone LTF-2, BioXcell, US) or 100 μg anti-mouse PD-L1 (clone 10F.9G2, BioXcell, US) were administered intraperitoneally (i.p.) on days 1,4,7,10 and 13 after the start of treatment. Mice were euthanized when pre-defined humane endpoint criteria were reached: Passiveness and other behavioral irregularities, a >15% weight loss within two days, a tumor size >1500 mm³ or tumor ulceration or infection. Mice that reached a tumor size >1500 mm³ served as the death endpoint for survival analysis. Tumor growth inhibition was calculated as,

$$Tumor growth inhibition ( TGI ) = \frac{Tumor {size}_{IgG control} - Tumor {size}_{t r e a tment group}}{Tumor {size}_{IgG control}} \times 100 \% \qquad ( Equation 5 )$$

### MC38 Model

The MC38 coloncarcinoma cell line (Catalogue number ENH204-FP) was purchased from Kerafast (US). MC38 cells were grown in high-glucose Dulbecco's Modified Eagle's Medium (DMEM; Sigma Aldrich) supplemented with 10% fetal bovine serum (FBS; Sigma Aldrich) and cells were split with EDTA/Trypsin when they reached 85% confluence.

MC38 model: Combination study of pIC-AL and ICI:

5×10⁵ MC38 tumor cells (in 100 μL DMEM without FBS) were first injected subcutaneously into the right flank of female C57BL/6 mice. Tumors were detected after 5-7 days after implementation. Tumor size was monitored daily using a digital caliper and the following formula: Volume (mm³)=(length×with²)/2. After 7 days of tumor implementation (day 0 of start of treatment), the tumors reached an average tumor volume of 100 mm³.

Mice were randomized into 4 treatment groups: PBS/IgG (n=8), PBS/a-PD-L1 (n=8), poly (I:C) encapsulated in anionic liposomes (10 μg) (pIC-AL) (n=8) and poly(I:C) encapsulated in anionic liposomes (10 μg) (pIC-AL)/a-PD-L1 (n=8). PBS or encapsulated) were poly (I:C administered intravenously on days 0,3,6,9, 12 after the start of treatment. 100 μg of Rat IgG2b isotype control (clone LTF-2, BioXcell, US) or 100 μg anti-mouse PD-L1 (clone 10F.9G2, BioXcell, US) were administered intraperitoneally (i.p.) on days 1,4,7,10 and 13 after the start of treatment. Mice were euthanized when pre-defined humane endpoint criteria were reached: Passiveness and other behavioral irregularities, a ≥15% weight loss within two days, a tumor size >1500 mm³ or tumor ulceration or infection. Mice that reached a tumor size >1500 mm³ served as the death endpoint for survival analysis.

MC38 Model: Comparison Between pIC-AL and pIC-LNP:

5×10⁵ MC38 tumor cells (in 100 μL DMEM without FBS) were first injected subcutaneously into the right flank of female C57BL/6 mice. Tumors were detected after 5-7 days after implementation. Tumor size was monitored daily using a digital caliper and the following formula: Volume (mm³)=(length×with²)/2. After 7 days of tumor implementation (day 0 of start of treatment), the tumors reached an average tumor volume of 100 mm³.

Mice were randomized into 5 treatment groups: PBS/IgG (n=5), poly (I:C) encapsulated in anionic liposomes (10 μg) (piC-AL)/IgG (n=4), poly (I:C) encapsulated in LNP (10 μg) (pIC-LNP)/IgG (n=6), poly (I:C) encapsulated in anionic liposomes (10 μg) (pIC-AL)/a-PD-L1 (n=6) and poly (I:C) encapsulated in LNP (10 μg) (RNA-LNP)/a-PD-L1 (n=6). PBS or encapsulated poly (I:C) were administered intravenously on days 0 after the start of treatment. Blood samples were taken after 3 hours of IV injection for cytokine and chemokines measurements. 100 μg of Rat IgG2b isotype control (clone LTF-2, BioXcell, US) or 100 μg anti-mouse PD-L1 (clone 10F.9G2, BioXcell, US) were administered intraperitoneally (i.p.) on days 1 after the start of treatment. All the mice were euthanized after 3 days of start of treatment as 1/6 mice of RNA-LNP and 4/6 mice of RNA-LNP/ICI groups were found dead in their cages due to high toxicity. All the mice of RNA-LNP and RNA-LNP/ICI group reached pre-defined humane endpoint (≥15% weight loss within two days). After mice were killed by cervical dislocation, blood was drawn by cardiac puncture and collected in EDTA tubes. Next, liver, spleen, kidneys, lungs and heart organs were collected, immediately frozen in liquid nitrogen, and stored at −80° C. Frozen organs were sectioned (5 μm) in using a cryostat (Leica CM1950) and mounted on adhesive poly-l-lysine coated extra-white gelatinized glass slides (StarFrost). The slides were fixed with formalin, stained with hematoxylin (Gills no. 2) and eosin Y solutions (H&E staining) and analyzed using an Olympus BX50 microscope

### Flow Cytometry and Antibodies for MC38-CEA Tumor Microenvironment Experiment

MC38-CEA cells were harvested in DMEM high Glutamax 1 with 10% FCS, 100 units penicillin/ml, and 100 μg of streptomycin/ml, and 1% Na-Pyruvate. Expansion: 70%-90% confluent cultures were split routinely using trypsin/EDTA. 1.0×10⁶ MC38-CEA tumor cells in 100 μl PBS were implanted into the left mammary fat pad of each mouse. Tumor growth was monitored by caliper measurement throughout the study. After randomization on Day 6, treatment was initiated the same day. Anionic liposomes containing poly (I:C) (pIC-AL) were administered at a dose of 10 μg intravenously once on Days 0 and 2 after start of treatment (n=4) and compared to PBS control also given intravenously once on Days 0 and 2 after start of treatment (n=5). On Day 5 after start of treatment, the study was terminated, all animals were euthanized, and a necropsy was performed. During necropsy, tumor tissues were harvested, and wet weight and tumor volume determined. Additionally, a part of each tumor tissue was processed for flow cytometry analysis. Primary tumor tissues were collected, and wet weight and tumor volume determined. Tumor tissue samples of 200-300 mg were selected and processed for flow cytometry: Primary tumor material (approx. 200 mg) was disrupted using gentleMACS™ C Tubes containing the enzyme mix of the Tumor Dissociation Kit according to the manufacturer instructions (Miltenyi Biotec, Germany). Following this, erythrocytes were removed with the Red Blood Cell Lysis Solution (Miltenyi Biotec, Germany). Obtained single cell suspensions from tumors were counted and dispensed in 96 well plates, if possible, at 3×10⁶ cells/well. Samples were incubated with 50 μl/well Fc block (anti-Mouse CD16/CD32, 1:50) for 15 min in FACS buffer. Then a 2× concentrated master antibody mix (fixable viability stain, CD3 (clone: 145-2C11, Biolegend), CD4 (clone: GK1.5, BD Biosciences), CD8a (clone: 53-6.7, BD Biosciences), CD45 (clone 30-F11, BD Biosciences), CD25 (clone: PC61, BD Biosciences), CD11b (clone: M1/70 Biolegend, Ly6C (AL-21 BD Biosciences), Ly6G (clone: 1A8, Biolegend, F4/80 (clone: T45-2342, BD Biosciences, CD11c (clone: HL3, BD Biosciences), MHC class II (clone: M5/114.15.2 Biolegend, CD206 (MR5D3), BD Biosciences, CD335 (clone: 29A1.4 Biolegend, CD49b (clone: HMa2, BD Biosciences, B220 (clone: RA3-6B2, BD Biosciences) was added to each well (50 μl) and incubated for 30 min in the dark. After washing, intracellular staining was primed by adding 100 μl fix/perm buffer (one-part fixation/permeabilization concentrate to three parts fixation/permeabilization diluent) for 30 min. After centrifugation at 840 g, the cell pellet was resuspended in 1× permeabilization buffer containing the anti-FoxP3 antibody and incubated for 30 min in the dark. After washing with 1× permeabilization buffer, cells were washed with FACS buffer. The cells were resuspended in FACS buffer containing counting beads and kept at 4° C. in the dark until analysis. The samples were analysed by flow cytometry using a LSR Fortessa (Becton Dickinson).

Tumor-infiltrating leukocytes (TILs) were quantified as the sum of B cells (CD45+CD11b-CD3-B220+), CD4+ T cells (CD45+, CD11b-, CD3+, CD4+, CD8−), and CD8 T cells (CD45+, CD11b-, CD3+, CD4—, CD8+). Myeloid-derived suppressor cells (MDSC) were identified as the sum of monocytic-MDSCs (M-MDSCs) (CD45+, CD11b+, F4/80-, Ly6C+, Ly6G-) and granulocytes (CD45+, CD11b+, F4/80-, Ly6G+).

### Orthotopic Hepa 1-6 Hepatocellular Carcinoma (HCC) Model

The Hepa 1-6 hepatocellular carcinoma (HCC) cell line was kindly gifted by Dr Piter Bosma (Tygat Institute for Liver and Intestinal Research, AMC, The Nethelands). Hepa 1-6 cells were grown in high-glucose Dulbecco's Modified Eagle's Medium (DMEM; Sigma Aldrich) supplemented with 10% fetal bovine serum (FBS; Sigma Aldrich) and cells were split with EDTA/Trypsin (Sigma Aldrich) when they reached 85% confluence.

Female C57BL/6 mice were used to develop the orthotopic HCC tumor model at NKI, The Netherlands. The operations were performed under isoflurane anaesthesia (3-4% induction, 2% maintenance). A middle incision of the abdomen was made, and the mesenteric vein was exposed. HCC tumor cells (Hepa 1-6:5×10⁶ cells/mouse) in 100 μl of PBS were injected via the mesenteric vein into the mouse liver. The injection site was covered by a cotton ball for 2-3 min to stop bleeding, before closing and sewing up the abdominal cavity. The mice normally recovered soon after operation. Tumor growth was monitored weekly by MRI imaging. Mice (n=5) that had detectable tumors 4 weeks were divided in two groups: 1) Control (n=2) and 2) pIC-AL (10 μg) (n=3). Treatment (PBS or pIC-AL was administered two times per week for 4 weeks. Tumor growths were followed up until reached humane end point up to 8 weeks after start of treatment.

MC38 Model: Comparison Between mRNA-AL and mRNA-LNP:

Female C57BL/6 mice were IV injected with PBS, anionic liposomes containing Fluc-mRNA (mRNA-AL) or lipid nanoparticles containing Fluc-mRNA (mRNA-LNP). Blood samples were taken in EDTA tubes after 3 hours of IV injection for cytokine and chemokines measurements. After 24 hours, mice were sacrificed, and spleens were collected. Spleens were placed on a 70-micron cell strainer, mashed using a syringe plunger and washed with 5 mL of ice-cold RPMI medium (Gibco, ThermoFisher Scientific). The resulting single-cell suspension was centrifuged at 300×g for 5 min at 4° C. and the supernatant was discarded. Erythrocytes were lysed by resuspending the pellet in 1 mL of ice-cold ammonium-chloride-potassium (ACK) lysis buffer and incubated for 2-3 min. After lysis, 9 mL of ice-cold medium was added to the cell-lysis buffer suspension and centrifuged at 300×g for 5 min at 4 degrees. The pellet was resuspended in 1 mL of medium, the cells were counted with trypan blue, and 600 000 cells was plated per well into 96-well V-bottom plate. Cells were washed once with PBS buffer before proceeding with flow cytometry staining. Plate was centrifuged at 300×g for 5 min at 4° C. and the supernatant was discarded. 25 μl of Fc-blocking antibodies was added per well and incubated for 5 min on ice. The staining was prepared in 75 μl of FACS buffer (1× DPBS (Gibco) supplemented with 2% FCS) per well containing the following fluorophore-conjugated antibodies and dilutions: Viakrome808 fixable viability dye (1:1000, Beckman Coulter), CD11b 450 (M1/70, Invitrogen), CD3e (145-2C11, BioLegend), B220 (RA3-6B2, BioLegend), Ly6G (1A8, BioLegend), XCR1 (ZET, BioLegend), CD25 (PC61, Biolegend), CD86 (GL-1, BD Biosciences), MHC II (M5/114.15.2, eBioscience), CD11c (N418, Miletnyi Biotec), CD80 (16-10A1, BioLegend), Ly6C (HK1.4, BioLegend). The cells were stained for 30 min at 4 degrees in the dark. The cells were washed three times with PBS before recording on flow cytometer Cytoflex (Beckamn Coulter).

### Statistical Analysis

Design expert® software (V13.0.12.0, Stat-Ease Inc. Minneapolis, USA) was used to design response surface and mixture experiments, data fitting and analysis. GraphPad Prism software (V.9.4.1, GraphPad Software LLC, California, USA) was used for a one-way analysis of variance (ANOVA) test with Tukey's post-test analysis, and a log-rank test as indicated in the figures. Error bars were expressed as mean±SEM, as indicated in the figure. P values were considered statistically significant when p<0.05.

### Results

Microfluidic Mixing Allows the Formation of Nucleic Acid Complexes with Anionic Lipids without Requiring Cationic Molecules

The present inventors developed a microfluidic process for encapsulating nucleic acid adjuvants in anionic liposomes. Microfluidics is a common technique employed to produce liposomes on a lab and industrial scale. In the microfluidics' nanoprecipitation method, there is a controlled mix regime of an organic-water miscible solvent (containing lipids) and an aqueous solvent. The diffusion of organic-water miscible solvent, preferably ethanol, into the aqueous phase reduces the lipid's solubility limit. Consequently, lipid starts to precipitate, growing from intermediate structures into liposomes. For the encapsulation of RNA into lipid nanoparticles, prior art formulations generally include a cationic lipid that facilitates the entrapment of the nucleic acids due to electrostatic interactions.

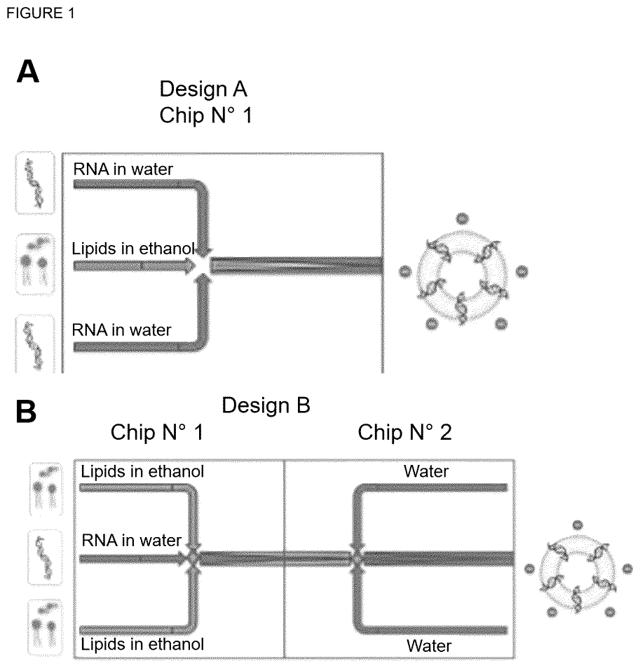

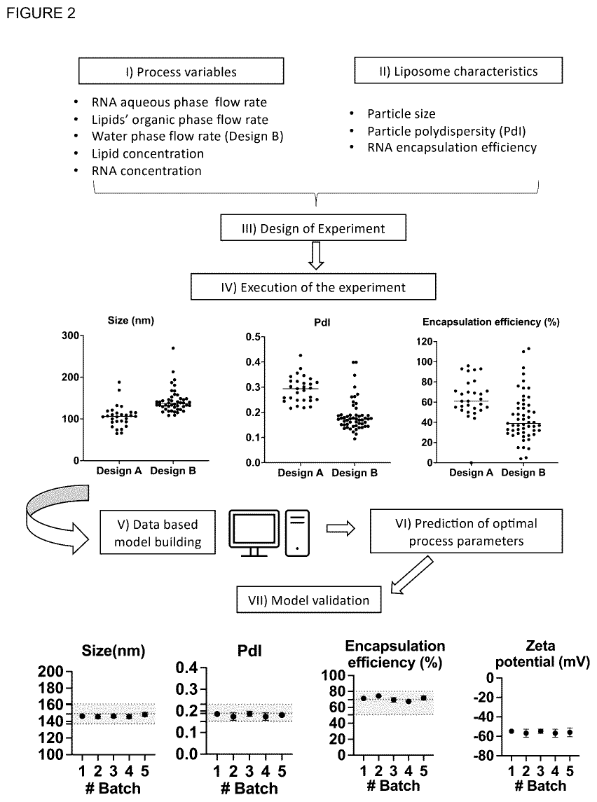

The present inventors started employing a fixed lipid composition of [EPC: EPG: Cholesterol (3:1:2 molar ratio)] yielding anionic liposomes. Surprisingly, during the proof of concept, the inventors observed that it is possible to encapsulate poly (I:C) (further referred to RNA), a double-stranded RNA model adjuvant, in anionic liposomes, e.g. by microfluidics. Notably, this was possible without using positively charged lipids, polymers, or cations. RNA encapsulation was feasible by employing diverse microfluidics chip setups. The inventors focused on two, named in this work as Design A and Design B (FIG. 1). To understand the mechanism behind this novel process and to obtain a formulation, the inventors decided to perform a design of experiment (FIG. 2). Following this approach the inventors identified the flow rates, lipid concentration and RNA concentration as variables that may relate to the characteristics of the liposomes: particle mean size, polydispersity (Pdl) and encapsulation efficiency. The inventors used a central composite design that allows building response surface models (RSM) for process optimization (FIG. 17 and FIG. 18). After preparing the samples for Design A (n=30) and Design B (n=53), the inventors built the response surface models (RSM) and analysed which process parameters were important for the formation of anionic liposomes containing RNA. The organic and aqueous flow rates can be used to vary the particle characteristics (size and PDI) while RNA encapsulation efficiency was affected by RNA concentration in Design A and B.

The present inventors identified important process parameters for anionic liposomes containing RNA. In both designs A and B, ethanol concentration during the mixing between RNA and lipids could be used to vary particle size and PDI. An increase in ethanol concentration led to bigger particle size and lower PDI. This is a possible indication that hydrophobic interactions may be determining the mechanism for the encapsulation of RNA in the anionic liposomes: At higher ethanol concentrations, RNA changes its conformation, exposing hydrophobic nucleotide bases that can interact with lipid tails, resulting in quite monodisperse nanoparticles. Furthermore, the inventors observed two advantages of design B over design A: The additional external aqueous phase allows selective particle size control without modifying the RNA/lipid ratio. Additionally, polydispersity is lower than 0.3 in most conditions tested, as required for pharmaceutical liposome products. Thus, the present inventors selected design B to produce anionic liposomes containing RNA.

### Microfluidics Process Optimisation and Model Validation

The RSMs employed not only allowed the inventors to understand the impact of each process variable on particle characteristics but also to optimise them for application. Within the space of design B, the inventors screened the process parameters for having suitable size, polydispersity, and encapsulation efficiency. The inventors set a target size of 150 nm for an optimised spleen targeting and manufacturability, as all intravenous injectable formulations which contain thermo-labile components are sterilized using a filter with a mean pore of 220 nm. Additionally, the target polydispersity was set as minimal as possible and the encapsulation efficiency as maximum as possible. The RSMs suggested a flow rate ratio of 1:3:4.4, 5 mM lipid concentration, and 500 μg/mL RNA concentration.

The inventors performed five independent batches in two days to verify optimal conditions and validate the RSMs (FIG. 2 VII). The experimental and predicted values were 149 nm and 150 nm for the particle size, 0.188 and 0.191 for the polydispersity index and the encapsulation efficiency were 69,6% and 65,8% for the experimental and predicted values, respectively. Therefore, particle size, polydispersity index and RNA encapsulation efficiency could be accurately predicted by the RSMs. Interestingly, anionic liposomes containing RNA had an average zeta potential of −56.7 mV, indicating a strong negative charge on the surface of the particles. Finally, the preparation process showed a remarkable reproducibility, required for clinical production.

### Impact of Mixing Flow Pattern on the Encapsulation of RNA in Anionic Liposomes.

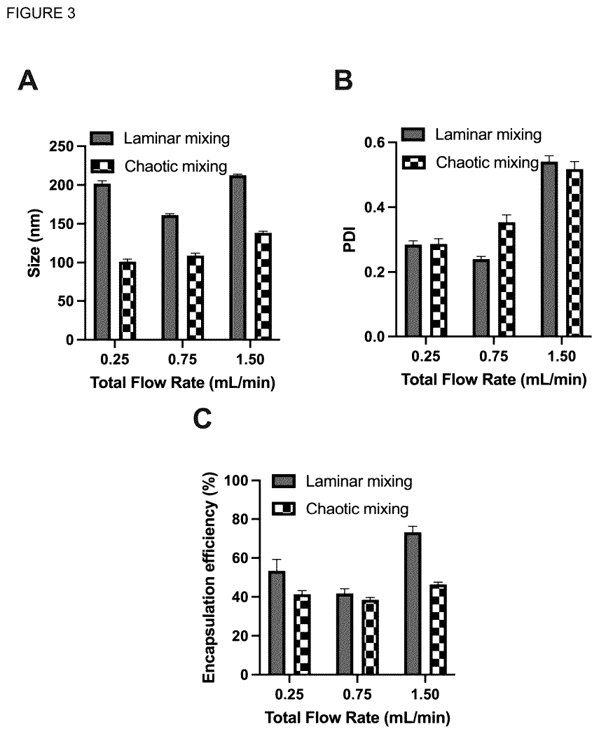

During the execution of the DoEs described above, the organic and aqueous flows containing lipids and RNA, respectively, were mixed using a laminar flow pattern. In this regime, characterized by a low Reynold number (Re<2000), the mixing relies on the passive diffusion of the molecules from a high concentration to a lower concentration domain. The mixing conditions by inter-diffusion depend strongly on the flow rate and the local geometry of the microfluidics mixer. Applying obstacles and baffles in the mixing chamber generates chaotic advection, and the fluids and molecules are rapidly mixed even if the Re number is smaller than 2000. In a staggered herringbone micromixer (chaotic advection micromixer), the mixing rate positively correlates with the number of obstacles and the total flow rates. The increased mixing rate has been reported beneficial for forming RNA-LNPs, leading to smaller and monodisperse particles. Here, the inventors studied the impact of the mixing regimen by including a chaotic micromixer and increasing the total flow rate on the pharmaceutical characteristics of anionic liposomes containing RNA (FIG. 3). The inclusion of chaotic micromixers leads to smaller nanoparticles. Surprisingly, the staggered herringbone micromixer does not improve the PDI, and higher total flow rates lead to higher PDI. These findings are the opposite of what has been observed for RNA-LNPs. Finally, the encapsulation efficiency tends to be higher in the laminar mixing. In summary, employing a chaotic mixer allows the production of smaller particles without an additional improvement in PDI and encapsulation efficiency of RNA in anionic liposomes.

### Formulation Optimisation Employing a Mixture Design

To understand the role of the lipid components (egg phosphatidylcholine EPC, egg phosphatidylglycerol EPG, cholesterol) on the characteristics and biological activity of anionic liposomes containing RNA, the inventors performed an I-optimal mixture design experiment (n=17). In this experiment, the inventors modified the relative proportion of the lipids ingredients while conserving the optimized process parameters (flow rates, lipid, and RNA concentration) described in under “Microfluidics process optimisation and model validation” (FIG. 16F). Statistically significant models were obtained for size (p=0.0005, R2=0.88), PDI (p<0.0001, R2=0.87), zeta potential (p=0.01, R2=0.47), encapsulation efficiency (p=0.005, R2=0.69) and TLR3 activation in HEK 293T TLR3+ cells (p=0.003, R2=0.82).

### Impact of the Lipid Components on Particle Characteristics

Particle size distribution was between 50 and 200 nm, (except for pure cholesterol-RNA complexes with a mean size of 800 nm (FIG. 16A)). These results show that neutral (EPC) and/or anionic (EPG) phospholipids were important to assemble RNA nanoparticles. The proportion of anionic lipid EPG has a role in particle size, PDI, and encapsulation efficiency: An overall trend of smaller particles and higher PDI was observed at a higher proportion of anionic EPG (FIGS. 16B and 16C). Additionally, the encapsulation efficiency was negatively correlated with EPG proportion, showing a potential incompatibility of anionic lipids and RNA (FIG. 16D). Interestingly, a statistically significant interaction was found between cholesterol and EPG. Anionic liposomes enriched with cholesterol tend to have lower PDI and higher encapsulation efficiency. This interaction shows that cholesterol is important in stabilising anionic liposomes containing RNA. Cholesterol induces changes in the lipid packaging in the bilayers, promoting the formation of lipid domains. Including cholesterol in the bilayer may stabilize lipid segregation, avoiding the electrostatic interactions between RNA and anionic lipids in the bilayer. Surprisingly, the binary combination of EPG and cholesterol resulted in low PDI and high encapsulation efficiency of RNA.

Regarding zeta potential, all the nanoparticles had a negatively charged surface. The increase in EPG proportion leads to a more anionic surface. The inclusion of EPG was important for colloid stability. Formulations without EPG tended to aggregate after a few days of storage.

### Impact of the Lipid Components on the Biological Activity of Anionic Liposomes Containing Poly (I:C)

The biological functionality of the formulations prepared in the mixture design experiment was evaluated in vitro using the HEK-Blue™-hTLR3 reporter cell line. Poly (I:C) is an agonist of the TLR3 receptor. This cell line is stably transfected with the human TLR3 gene and an inducible SEAP (secreted embryonic alkaline phosphatase) reporter gene. TLR3 is a receptor located in the endosomes of immune cells and is responsible for recognising extracellular dsRNA such as poly (I:C) that has been endocytosed. Its activation triggers an immune response. The inventors wanted to know if the lipids components of the liposomes may impact poly (I:C) recognition by TLR3.

The induction of the reporter gene was optimal when EPC, EPG and Cholesterol proportion was close to the standard formulation (EPC: EPG: Cholesterol=3:1:2 molar ratio). Additionally, lower activation of TLR3 was observed in the nanoparticles that had a higher proportion of EPG (FIG. 16E). The effect of EPG on encapsulated poly (I:C) biological activity may be explained by differential nanoparticle uptake or by a potential interference in the interaction between poly (I:C) and TLR3. The inventors assessed particle uptake by HEK-Blue™ h-TLR3, measuring by flow cytometry the fluorescence intensity of DiD, a fluorescent probe incorporated in the anionic liposome bilayer. Particle uptake was enhanced at higher EPG concentrations and was not positively correlated with TLR3 activation. Thus, an increased proportion of EPG may lower TLR3 activation by poly I:C. Indeed, dsRNA is recognized by histidine-rich regions of TLR3. In the late endosomes, pH is acidic and the histidine residues are protonated, promoting attractive electrostatic interaction between TLR3 and RNA phosphate groups. Above certain levels of anionic lipid molar ratio (%), EPG may influence this electrostatic interaction.

Finally, the present inventors assessed the anionic liposome uptake and activation by spleen conventional type 1 dendritic cells (cDC1s) ex vivo. This is a cell population that strongly expresses TLR3 and in the presence of poly (I:C), upregulates maturation markers. In this experiment, the inventors wanted to understand the impact of EPG concentration on particle uptake and maturation by cDC1. The inventors selected five formulations containing an increasing concentration of EPG (0, 4, 17, 33, 67% molar ratio), decreasing concentration of EPC and constant concentration of cholesterol (33% molar ratio). As seen with the HEK293 TLR3⁺ cell line, an increased molar ratio of EPG enhances nanoparticle uptake by cDC1 cells (FIG. 12A). Liposomes that contained 0, 4 and 17 mol % of EPG lead to a significant upregulation of CD80⁺ costimulatory markers (FIG. 12B) while the increase of EPG proportion to 33 and 66 mol % reduces poly (I:C) immunostimulant properties.

Molecular Weight Characterisation of Encapsulated dsRNA and Particle Size Distribution after Plasma Incubation.

Poly I:C is a non-coding synthetic dsRNA polymer. The high molecular weight poly (I:C) employed in this study has a declared average size of 1 kb to 8 kb. Encapsulation of the dsRNAs employing microfluidics may differentially enrich specific molecular size fractions. The inventors used size-exclusion chromatography (SEC) coupled with multiangle light scattering and ultraviolet detection (SEC/MALS/UV) to characterise the molecular weight of the dsRNA before and after encapsulation in anionic liposomes.

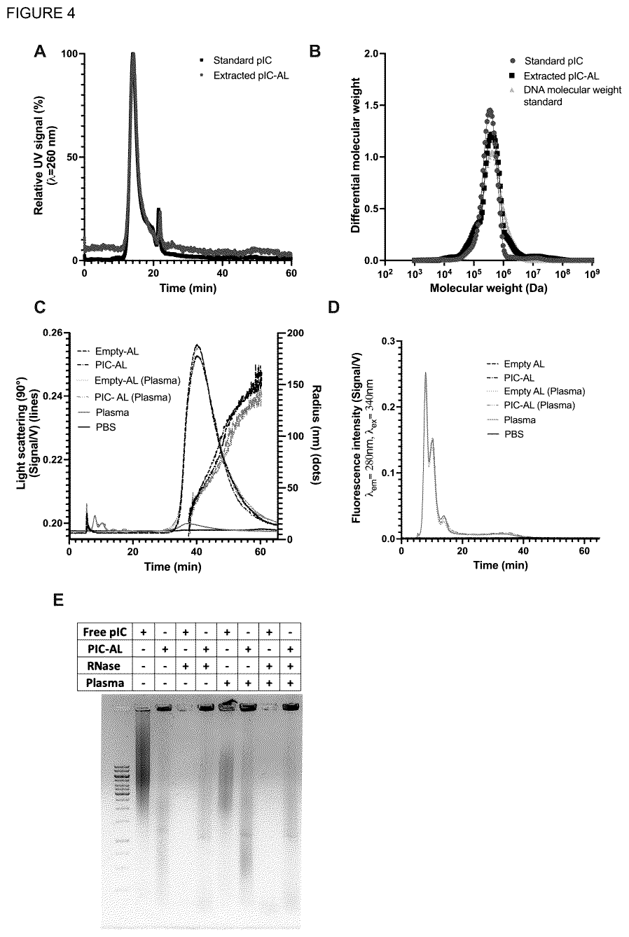

The SEC-MALS-UV analysis shows that the initial free poly (I:C) and poly(I:C) encapsulated in anionic liposomes had a similar molecular weight ranging from 104 to 107 Da (approximately 10 to 104 base pairs) (FIG. 4B). A slight enrichment of high molecular weight fractions was observed for the encapsulated poly (I:C). Comparable retention time and UV absorbance profile were observed for soluble and encapsulated poly (I:C) (FIG. 4A). These results show that the nucleic acids' molecular weight is not a limiting factor for their encapsulation in anionic liposomes.

Liposome drug products need to show physical stability in a relevant physiological medium. Formulations were incubated with PBS or 20% human plasma at 37° C. for two hours and subjected the samples to an asymmetrical flow field-flow fractionation (AF4) coupled with multi-angle light scattering (MALS), UV and fluorescence detectors. Empty and poly(I:C) containing anionic liposomes showed similar elution and light scattering profiles and particle size distribution (FIG. 4C). Samples incubated with human plasma showed overlapping light scattering curve and similar particle size distribution to the respectively liposomes incubated in PBS. Furthermore, free poly (I:C) and encapsulated poly (I:C) in anionic liposomes were treated with RNase Ill in reaction buffer or reaction buffer supplemented with 20% human plasma at 37° C. for two hours and later analyzed in a 1% agarose gel electrophoresis (FIG. 4E). Free poly (I:C) was completely degraded by RNase III. Encapsulated poly (I:C) presented similar electrophoretic patterns in the presence or absence of RNase III and/or human plasma, indicating that anionic liposomes retain and protect nucleic acids.

These results show that anionic liposomes containing RNA remain physically stable and protect nucleic acids from RNase degradation when exposed to human plasma at physiological temperature.

Encapsulation of RNA in Anionic Liposomes is Possible while Maintaining the Lipid Bilayer Structure

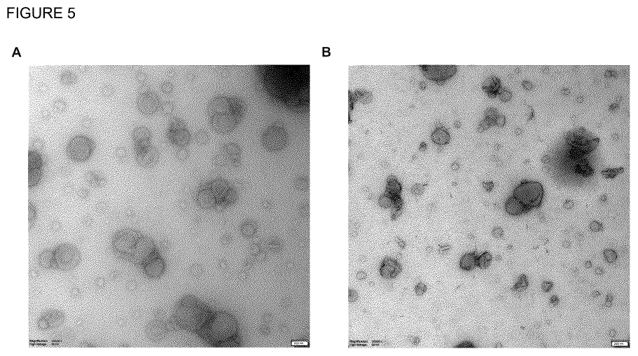

Firstly, the inventors assessed the structure of empty and RNA-containing anionic liposomes using negative staining transmission electronic microscopy (TEM). The inventors employed uranyl salts which bind to lipids and nucleic acids' phosphates, to create electron contrast in the samples. Empty nanoparticles had the typical liposome vesicle structure, with one or more lipid bilayers and an empty core (FIG. 5A). On the other side, the incorporation of RNA in the anionic nanoparticles changed their contrasting density, especially in the bilayers, due to the additional phosphate groups (FIG. 5B). One or more lipid bilayers were observable in the nanoparticles containing RNA.

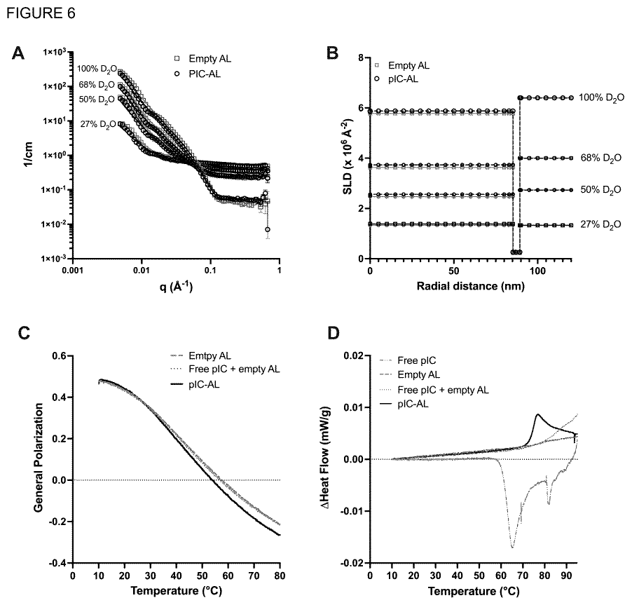

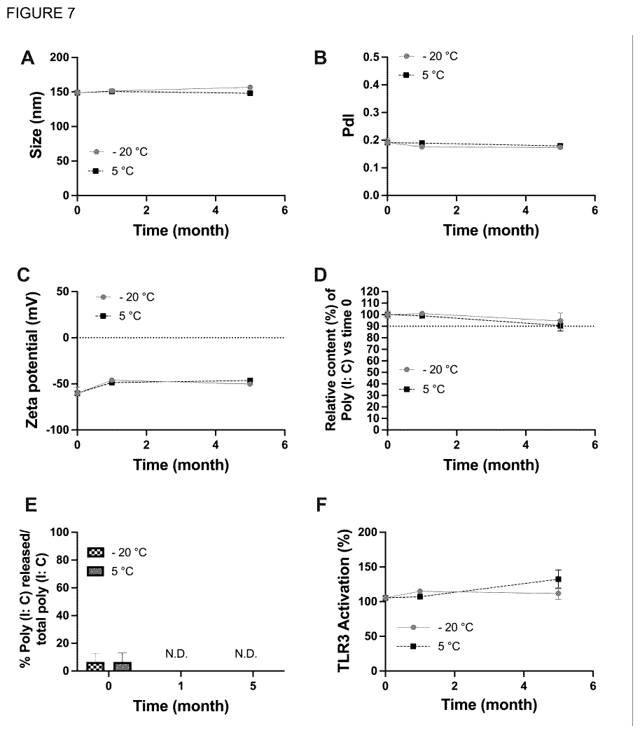

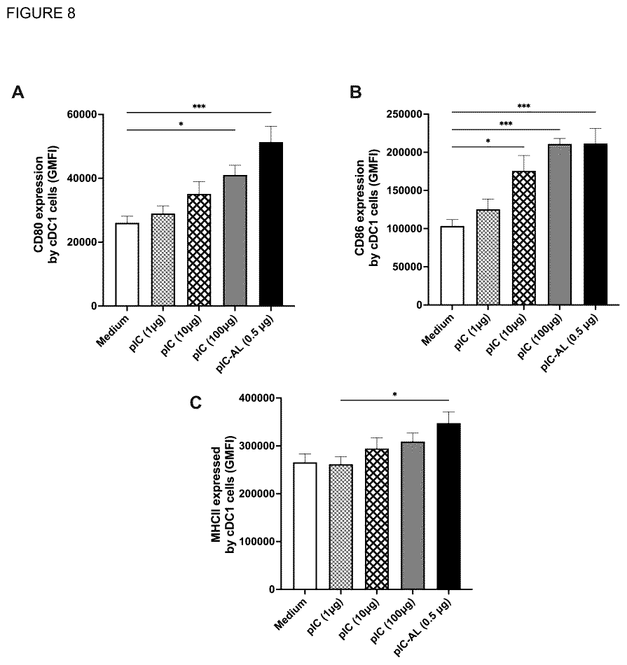

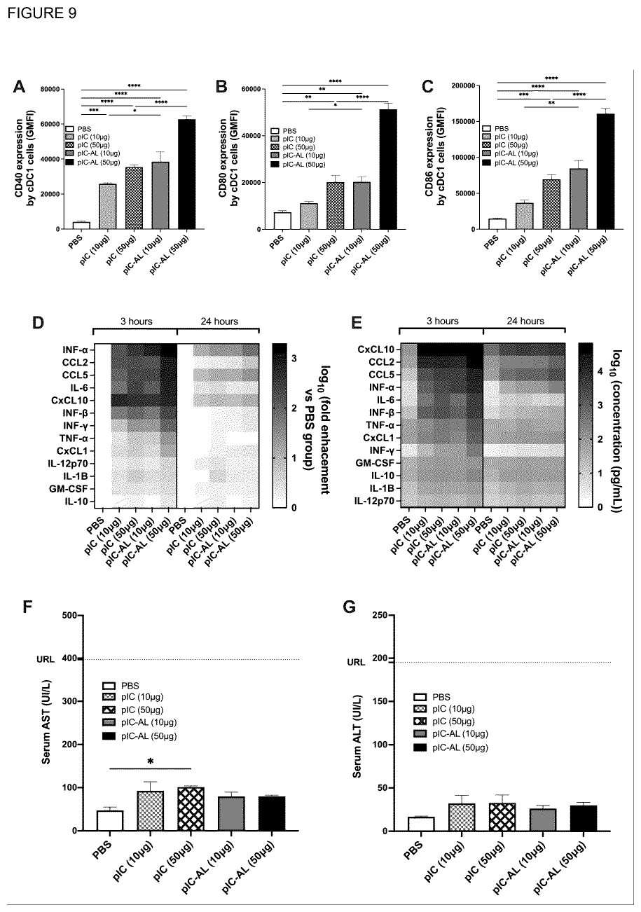

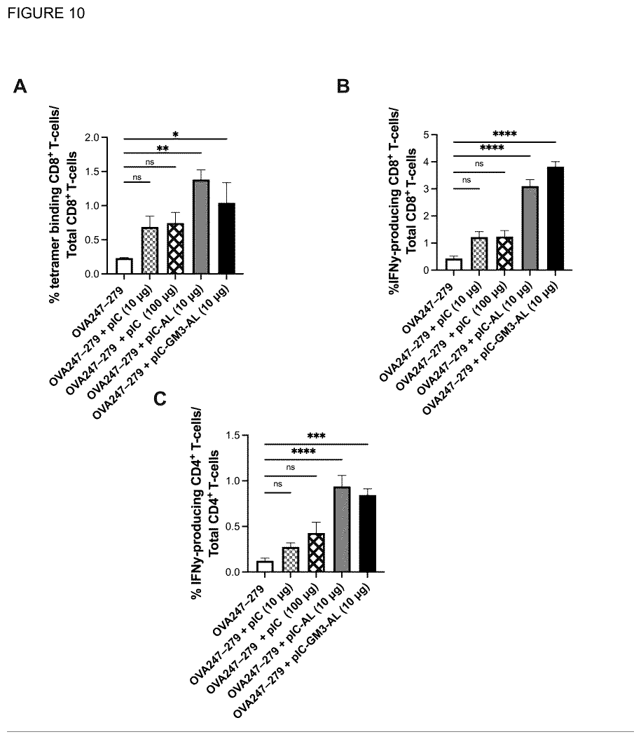

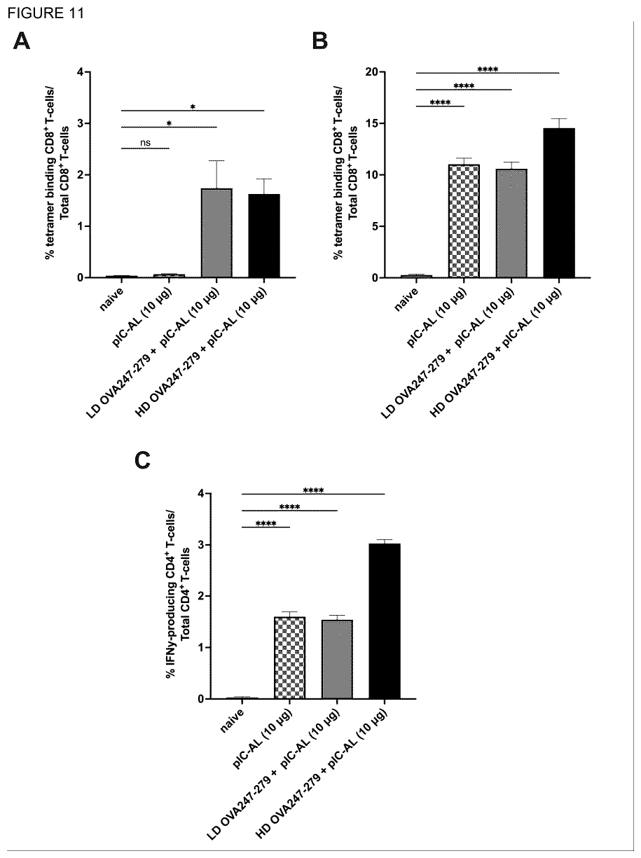

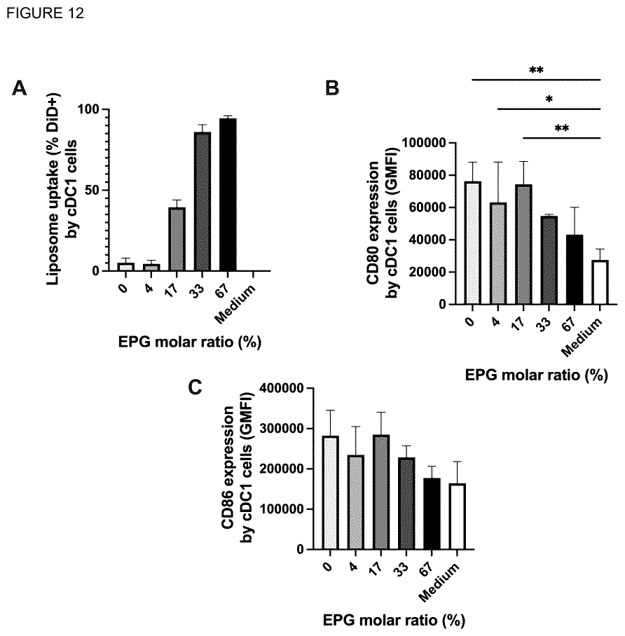

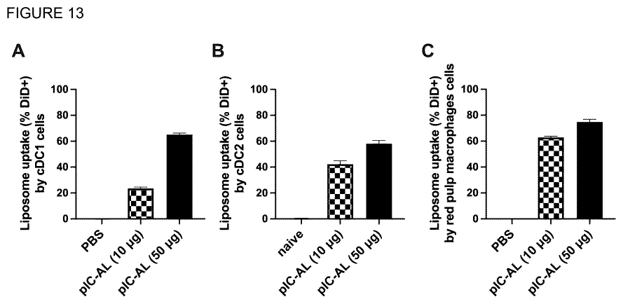

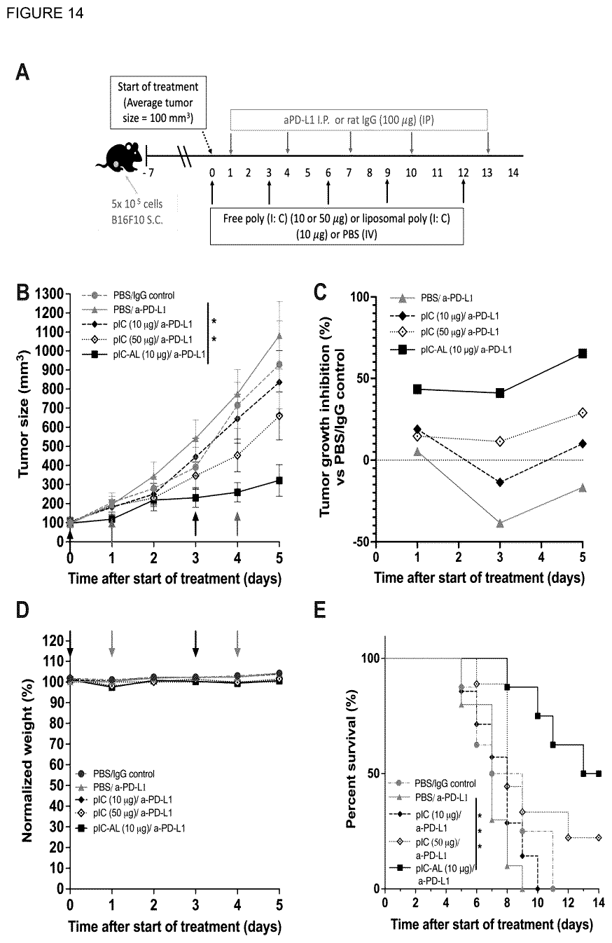

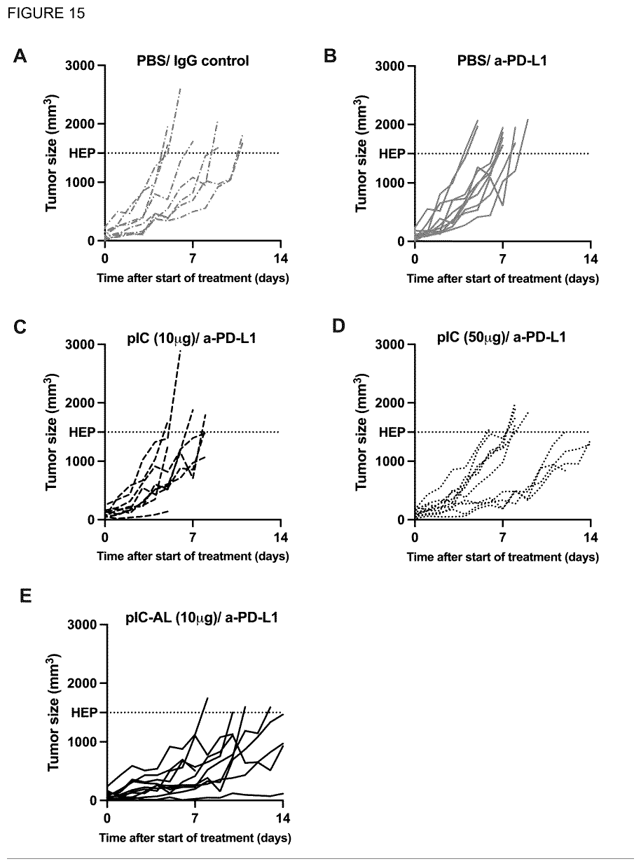

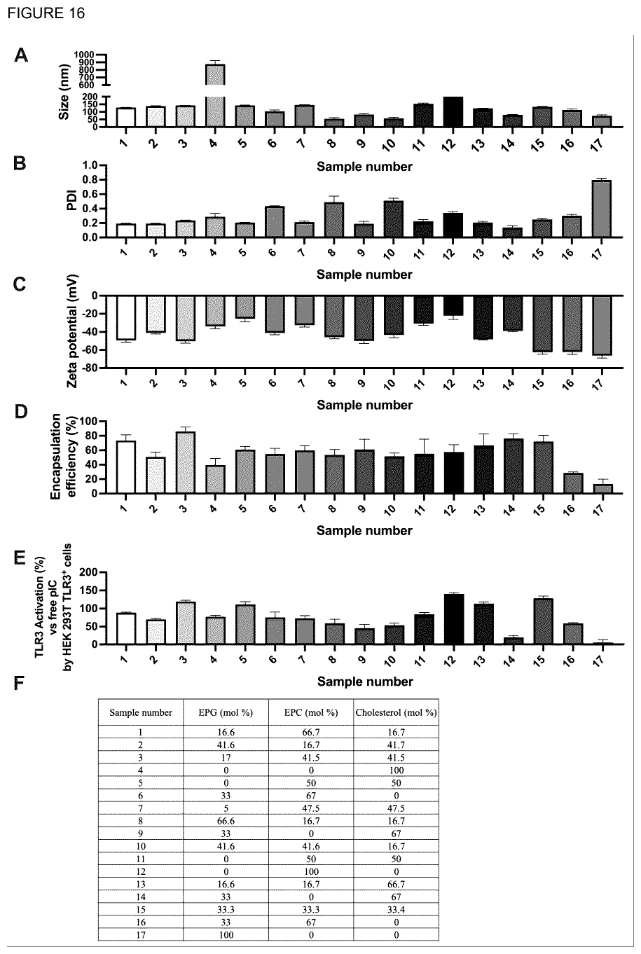

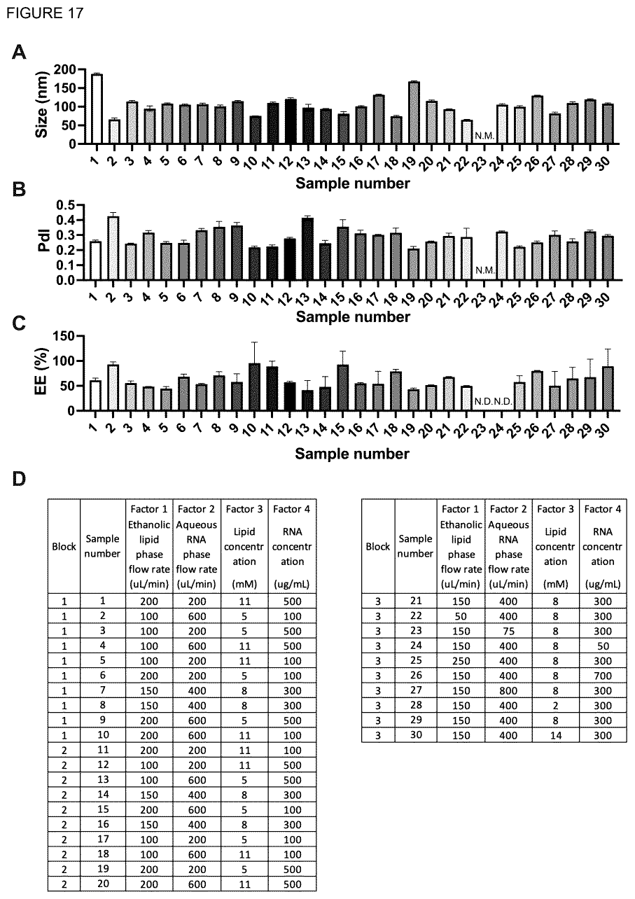

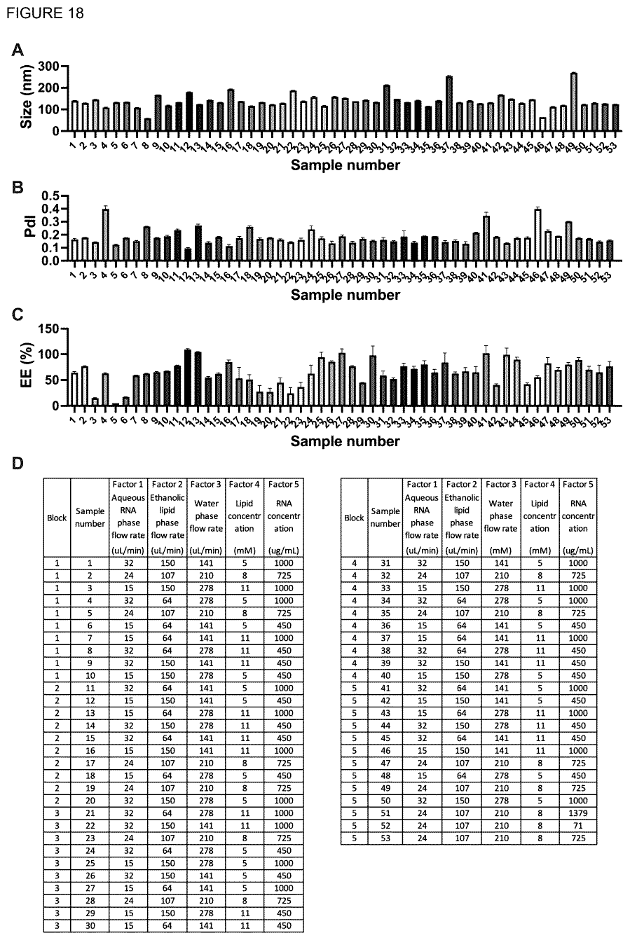

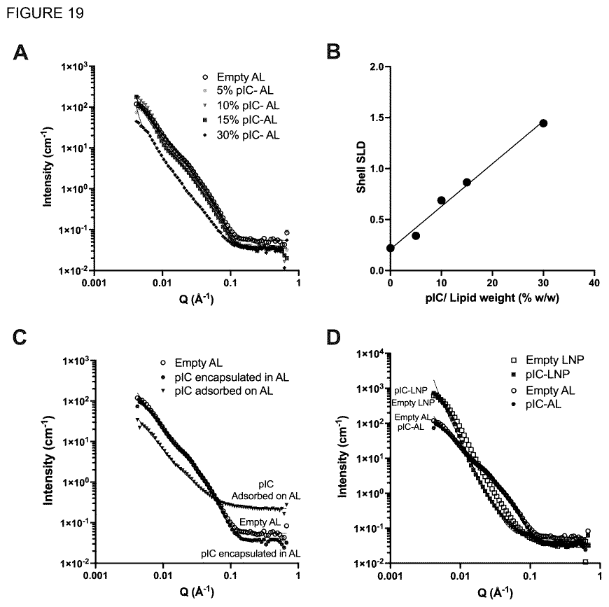

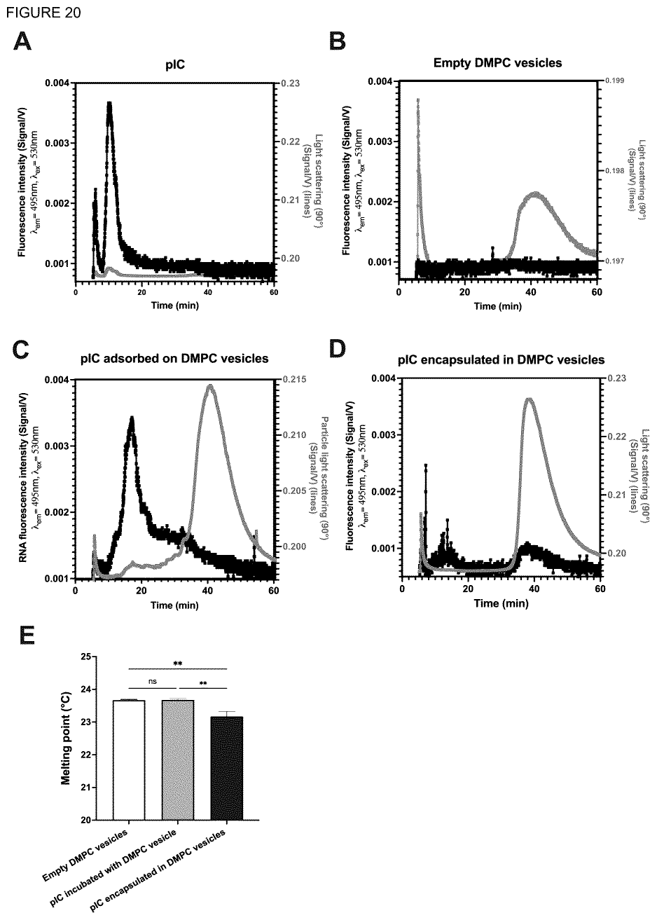

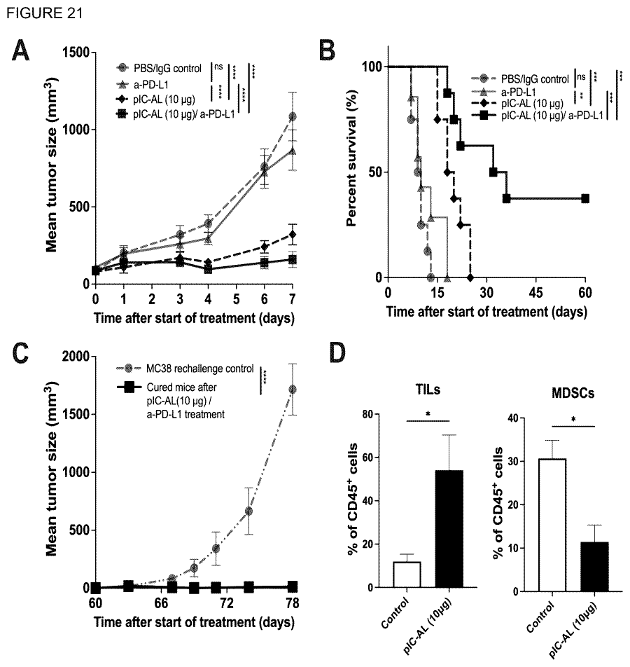

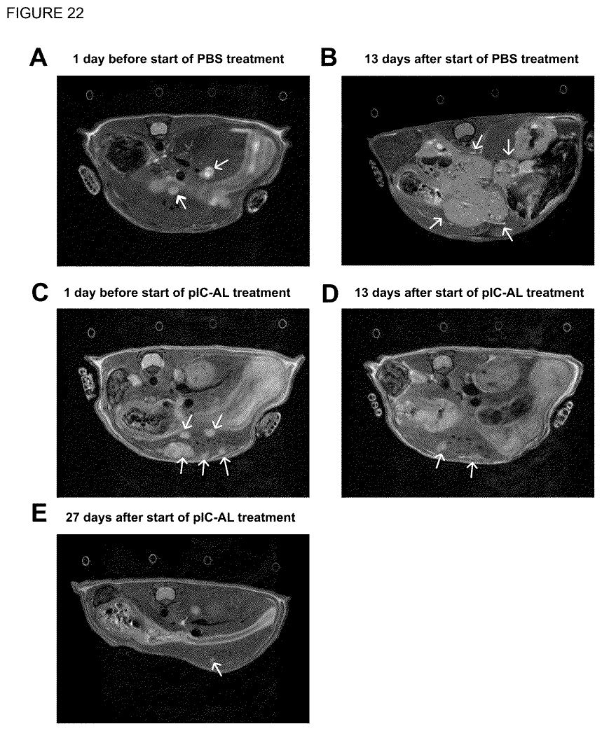

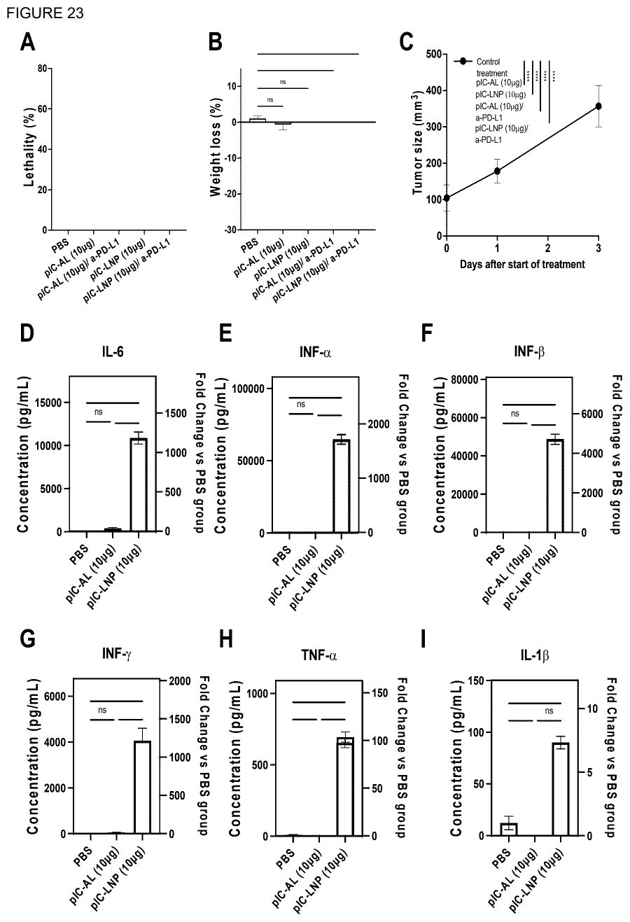

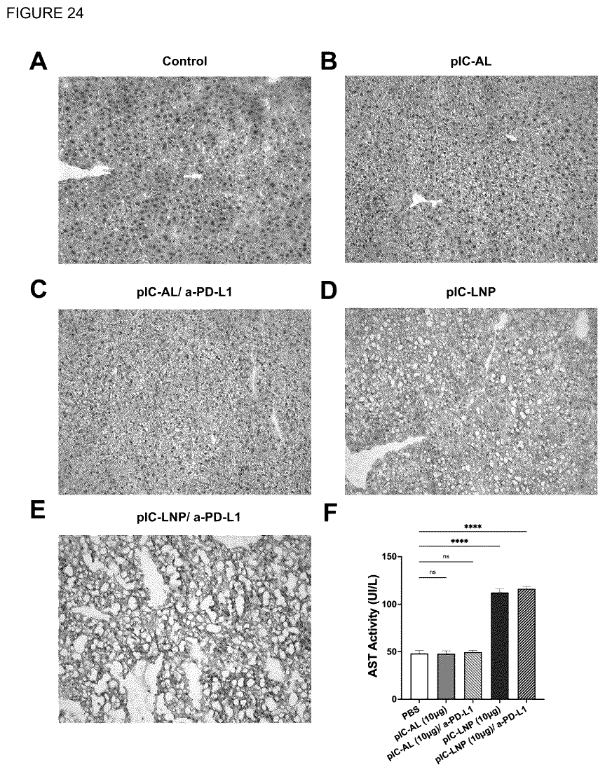

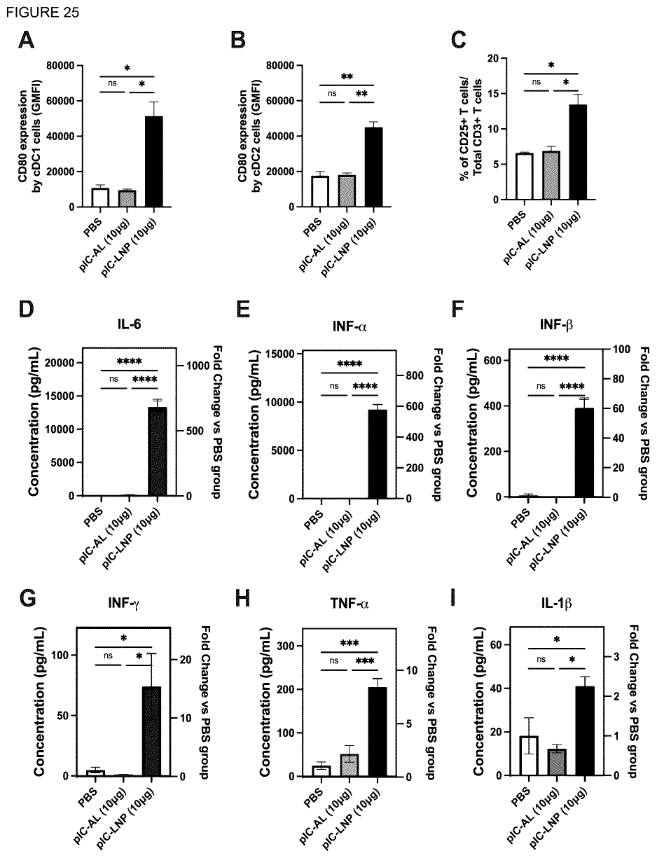

Secondly, the inventors used SANS with isotropic contrast variation and deuterated lipids to understand the mass distribution of empty and pIC-containing anionic liposomes (pIC-AL). The contrast variation is a technique for elucidation of the nanoparticle's external and internal structure. In this experiment, the samples were analyzed in 100/0%, 68/32%, 50/50% and 27/73% D₂O/H2O (FIG. 6A). In 68% D₂O/H₂O solvent, the contrast between solvent-accessible RNA and the solvent vanishes and only the scattering length density (SLD) of the lipids are detected.

A core-shell model was used to fit the measured neutron scattering curves, and a similar shell thickness about 4.1 nm were obtained for empty and pIC-AL (FIG. 6B). This shell tickness is compatible with that of a phospholipid bilayer. The scattering length density (SLD) of the shell was higher in pIC-AL (0.26) than in empty liposomes (0.22), suggesting that RNA is present in the shell. Additionally, the SLD of the core of empty-AL and pIC-AL was close to the solvent in the contrast series. This indicates that 1) the solvent can freely diffuse through the shell into the core, 2) the core is mainly composed of aqueous solvent and 3) some residual lipids are forming internal vesicles. In the case of pIC-AL, some RNA is localized within the core, contributing to a slightly higher core SLD compared to empty-AL.

Thirdly, the inventors performed contrast series with samples containing one or more deuterated lipids (DPPCd62, POPCd31, DPPGd62, POPGd31, cholesterol d7) to understand the lipid distribution within the nanoparticles. All the samples containing deuterated lipids had an increase in the SLD of the shell and a slight increase in the core versus undeuterated anionic liposomes, confirming the main distribution of the neutral and anionic lipids as well as cholesterol in the lipid bilayers.

It is possible to calculate the content of solvent inside the core by knowing the scattering light density of the components and their volume fraction (Equation 1):

$$S L {D}_{c o r e} = S L {D}_{l i pids} * {V}_{l i p i d s} + S L {D}_{RNA} * {V}_{R N A} + S L {D}_{solvent} * {V}_{solvent} \qquad ( Equation 1 )$$

Calculations show that the core of empty-AL and pIC-AL is mainly water (90 and 91% v/v), in agreement with the structure of liposomes. RNA is a hydrophilic molecule, which can be dissolved in the aqueous core of the liposomes. In that case of being dissolved in the aqueous core, the pIC-AL resuspended in 68% D₂O would have a different scattering profile than in 100%, 50%, or 27% D₂O. Surprisingly, during the contrast series, no differences in the scattering profiles was observed for RNA-containing liposomes. This shows that the RNA may not be accessible for the aqueous solvent. Therefore, SANS data suggest that RNA may be associated with the lipids in the hydrophobic bilayers of the shell and internal vesicles rather than in a water soluble form in the aqueous core of the liposomes.

Thirdly, to confirm the hypothesis that RNA increases the SLD of the shell, the inventors repeated the SANS experiment, testing anionic liposomes with increasing concentration of encapsulated RNA: 0/100%, 5/95%, 10/90%, 15/85% and 30/70% (encapsulated RNA/lipid wight ratio (w/w %)) at 100% D₂O. Employing a core-shell model for fitting the experimental data, it was observed a direct correlation (R2=0.988, p=0.005) between the encapsulated RNA/nanoparticle lipid weight ratio and the SLD of the shell (FIGS. 19A and 19B). This indicates that the RNA is at least, partially localized within the lipid shell.

Fourthly, to understand whether the RNA is localized at the surface of the nanoparticle, as an adsorbed layer on the nanoparticles or is localized within the hydrophobic region of the lipid bilayer, the inventors compared the scattering profile of preformed empty nanoparticles incubated with externally added free RNA and nanoparticles with encapsulated RNA in 100% D₂O. As observed in FIG. 19C, the scattering pattern of encapsulated RNA and co-incubated RNA is different.
A. In case of pre formed nanoparticles incubated with externally added RNA, there is an increase of scattering at low Q (0.1 to 1 Å⁻¹) which indicates an increase in the background. The increase in background can be attributed to more hydrogen in the D₂O solvent following the addition of RNA, suggesting that the RNA is localized in the solvent rather than in the nanoparticles.
B. No appreciable increase in the background is observed in case of nanoparticles with encapsulated RNA, suggesting that the RNA is localized in the nanoparticles rather than in the solvent.

Fithly, the inventors compare the scattering profile of anionic liposomes and the current state-of-art for RNA delivery, lipid nanoparticles (LNP), by analyzing empty nanoparticles and 5% w/w containing RNA nanoparticles. As described before, the process of encapsulation of RNA in anionic liposomes does not change the structure of the nanoparticles, observing a comparable scattering profile (FIG. 19D). However, the encapsulation of RNA in LNP modifies the scattering profile, indicating a rearrangement of the internal structure, as has been reported previously by Arteta et al. Proceedings of the National Academy of Sciences 115, E3351-E3360 (2018)). The rearregement observed in LNP may be explained by the electrostatic interactions between the ionizable cationic lipid and the anionic RNA, which does not occurs in the anionic liposomes containing RNA. Therefore, this evidence suggest that anionic liposomes have a distinctive structure and the interaction with RNA is through a novel mechanism independent of the internal structure rearrangement observed in the LNP.

If RNA is truly embedded in the lipid bilayers by hydrophobic interactions, as SANS experiments suggest, the physical properties of the RNA and the lipids in the anionic liposomes containing RNA should change.

The inventors studied the changes in the lipid packaging in the bilayers of the anionic liposomes in the presence of free and encapsulated RNA over a range of temperatures (10° C.-80° C.) (FIG. 6C). Laurdan, a membrane-embedded fluorescent molecule, was employed to measure the relative levels of membrane lipids packaging and reported as generalised polarization (GP) parameter. Encapsulated RNA but not soluble RNA affects the lipid packaging order of the liposomes bilayer, resulting in a lower GP value during the heating ramp (FIG. 6C). The effect was specially observable anionic in liposomes with liquid order and disordered domains (DPPC: POPC: DPPG: POPG: Cholesterol (3:3:1:1:4)). These results confirm that encapsulated RNA interacts with the hydrophobic bilayer.

Poly (I:C) is a double-strand RNA that melts (the strands separate) at high temperatures. Using differential scanning calorimetry (DSC), the inventors characterized the thermally induced transitions of the structure of free and encapsulated poly (I:C) (FIG. 6D). Free poly (I:C) has a melting (endothermic) event at 65° C. In contrast, there is no melting event when poly (I:C) is encapsulated in liposomes or mixed with empty liposomes. The binding of poly (I:C) in the bilayers in the former case or to the surface of the liposomes in the latter case may inhibit the separation of the strands. Surprisingly, encapsulated poly (I:C) has an exothermic event at 77° C., which may be explained by the crystallization of the RNA in the hydrophobic environment of a liposome bilayer. Indeed, hydrophobic organic solvents are generally used for growing nucleic acid crystals.

Michanek et al. have previously reported that externally added nucleic acids to previously formed phospholipid lipid bilayers associate with the surface of the bilayers (Michanek et al (Biochimica et Biophysica Acta 1798 (2010) 829-838). By using quartz crystal microbalance with dissipation (QCM-D), Michanek et al. reported that tRNA adsorbs onto DOPC bilayers (14 mg/m²) and DPPC bilayers (6 mg/m²). However, the degree of adsorption is very low, as the measured difference of mass for the adsorption of tRNA to the bilayer corresponds to one tRNA molecule per 1700 DOPC lipids, and one tRNA per 4300 DPPC lipids. Additionally, the interaction of RNA on the bilayer surfaces is weak and reversible, as the adsorbed layer of tRNA was immediately removed by the rinsing with a10 mM NaBr solution. Moreover, Michanek et al. performed DSC studies with RNA adsorbed onto the surface of DMPC vesicles that allowed to describe how RNA interacts with the surface of DMPC bilayers. The melting point of DMPC vesicles was not affected when they were incubated with different types of nucleic acids (tRNA, ssDNA and dsDNA), indicating that there is no significant penetration of nucleic acids in the hydrophobic region of the bilayers. As described by Michael et al, the highly charged phosphate groups of nucleic acids are very unlikely to penetrate the apolar region of the lipid bilayers.

To further confirm the unexpected novelty of the present invention, the inventors employed the DSC technique for studying the type of association of RNA with DMPC bilayers in case of either adsorption on the surface or in case of encapsulation by the method described in the present invention. The association of RNA with the outer surface of empty DMPC vesicles does not induce a decrease of the melting point as described in the prior art, but the encapsulation of RNA according to the current invention does induce a significant melting point reduction (FIG. 20E). This finding together with the findings described above, strongly suggest that our RNA encapsulation methodology promotes an unforeseen association of the RNA into the apolar region of the lipid bilayers. Additionally, the inventors studied if the encapsulation of RNA in liposomes can overcome the current limitations of previous art regarding the weak and reversible interaction of RNA with outer bilayer surfaces. The inventors observed that the interaction of adsorbed RNA with bilayer surfaces is weak and reversible and a simple dilution with water is already enough to remove the adsorbed RNA from the surface of preformed DMPC vesicles. Surprisingly, when the RNA is encapsulated in DMPC liposomes by the method of the invention, there is no strong fluorescence signal between 5 and 20 minutes after start of the run, when RNA is expected to elute from AF4 channel. This indicate that the interaction between RNA and the liposomes is strong and not reversible, and dilution does not have a removal effect. Moreover, the fluorescence signal of RNA is weak when particles are eluted from AF4 channel (from 30 to 60 minutes of run), suggesting that the fluorescence is quenched because the RNA is inside the nanoparticle and not on the external surface. (FIG. 20A-D)

In summary, these results suggest that anionic liposomes containing RNA have an unexpected structure: Anionic liposomes have a core-shell structure with RNA embedded in the lipid bilayers and an almost empty aqueous core. The structure of pIC-AL is distinctive to the one reported for conventional RNA-LNPs. Arteta et al, have recently used SANS and SAXS techniques to characterise RNA-LNPs and showed that they have a different core-shell structure (Arteta et al. Proceedings of the National Academy of Sciences 115, E3351-E3360 (2018)). The shell is composed of DSPC and lipidated-PEG but without RNA while the core is composed of RNA and lipids that do not self-assemble into bilayers (ionizable cationic lipid and cholesterol), but rather a disordered hexagonal internal structure. The water content is remarkably smaller in RNA-LNP than in RNA-anionic liposomes (24% vs 91%) showing that the core of RNA-LNP is much more solid.

### Stability of Anionic Liposomes Containing Poly (I:C)

The mismanagement of the cold chain is one of the main reasons for the waste of vaccines. During the storage at 2-8° C., certain refrigerators like ice-lined refrigerators risk overcooling and freezing liquid formulations that lead to waste of the vials. Although most vaccines are freeze-sensitive, therefore, having a freeze-insensitive formulation is desirable from a supply point of view.

During the formulation development, the inventors performed stability studies of anionic liposomes containing poly (I:C). The lipid composition EPC: EPG: Cholesterol (3:1:2) was selected for the stability studies. The inventors studied the incorporation of sucrose, a sugar that is widely used as a cryoprotectant for preventing the aggregation of liposomes after freezing and freeze-drying them. The anionic liposomes containing poly (I:C) were resuspended in RNase-free 10% sucrose 20 mM citrate buffer (pH 6.5) and stored at −20° C. and 5° C. for 5 months. Particle size, polydispersity, zeta potential, total poly (I:C) content and % of poly (I:C) released and poly(I:C) activity in HEK TLR3⁺ was studied (FIG. 7A-F). Anionic liposomes containing poly (I:C) were physically stable without signs of particle aggregation for 5 months when they were stored at −20° C. and 5° C. (FIG. 7A-C). The total concentration of poly (I:C) slightly decrease after 5 months 5% at −20° C. and 9% at 5° C. (FIG. 7D). Although some released RNA was detected at time 0 (less than 10%), no additional release of RNA was detected after 1 month (FIG. 7E), indicating a strong interaction of encapsulated RNA with anionic liposomes. Furthermore, the biological activity in HEK TLR3+ cells did not decrease after 5 months of storage at −20° C. and 5° C. (FIG. 7F). These results show the stability of poly (I:C) encapsulated in anionic liposomes.

### Effects of the Incorporation of Poly (I:C) in Anionic Liposomes on the Maturation of Spleen Conventional Type 1 Dendritic Cells, Anti-Viral Cytokines Levels and Hepatic Toxicity.

The spleen is the main secondary lymphoid organ, containing multiple subsets of myeloid and dendritic cells (DCs). DCs are the initiators of the adaptive cellular immune response through the antigen capture, process, and presentation to naïve T cells. In the presence of adjuvants and antigens, DCs maturate and induce strong T cell responses. DCs are a heterogenous population consisting of multiple cell type subsets. Among them, conventional type 1 (cDC1) is characterised by the high expression of TLR 3 in the endosomes, enabling them to recognize double-strand RNA molecules like poly (I:C) adjuvant and the initiation of the adaptive immune response. cDC1s are of special interest in tumour immunology. It is shown that the number and activation of cDC1s is strongly positively correlated with improved survival in multiple human cancer types.

The inventors studied the immunostimulant properties of anionic liposomes towards splenic cDC1s. Incorporating poly (I:C) in anionic liposomes augments the maturation of spleen cDC1s ex vivo, reflected by an enhanced expression of CD80, CD86 and MHC-II surface markers (FIGS. 8A, B and C).

The inventors evaluated to what extent poly (I:C) containing anionic liposomes were able to mature cDC1 in vivo. Anionic liposomes containing poly (I:C) were found to be highly effective in inducing the maturation of cDC1s in a dose-dependent way after IV administration (FIG. 9A, B, C). Encapsulated poly (I:C) was superior to free poly (I:C) in inducing cDC1 maturation at 10 and 50 μg doses. Interestingly, 10 μg of encapsulated poly (I:C) induced a similar expression level of surface maturation markers (CD40, CD80 and CD86) than 50 μg of free poly (I:C). At 50 μg dose of poly (I:C) encapsulated, most spleen cDC1s, cDC2s and red pulp macrophages captured anionic liposomes containing poly (I:C) (FIGS. 13A, B and C). Additionally, the inventors studied the levels of serum cytokines at 3 and 24 hours after I.V. administration of soluble and encapsulated poly (I:C) (FIG. 9D-E). Cytokines are a family of small proteins that are crucial for the coordination of the immune response and are of interest for cancer immunotherapy. Indeed, cytokines such as interferon-alpha and IL-2 have been approved for cancer treatment. Anionic liposomes containing poly (I:C) were found to enhance the cytokine serum levels greatly, especially for interferons (α, β, and γ), chemokines (CCL2, CCL5, CxCL10) and interleukin-6 in comparison to free poly (I:C), in a dose-dependent fashion (FIG. 9D). These cytokines have anti-tumour proliferative activity, promote antigen priming enhance T-cell cytolytic activity (interferon-α), enhance endothelial permeability (IL-6) and promote immune cell infiltration into the tumour microenvironment (CCL2, CCL5, CxCL10). Additionally, they have anti-viral replication activity. The inventors also measured IL-10, an immunosuppresive cytokine that promotes tumour cell proliferation, and did not observe a significant effect on its level by liposomal poly (I:C). Finally, the inventors studied if the stronger immune response observed with encapsulated poly (I:C) could be correlated with enhanced toxicity. Poly (I:C) treatment (20 μg/g) had been used to simulate viral hepatitis in mice. The inflammation of the liver induces liver tissue damage and the release of hepatic enzymes such as ALT and AST. Remarkably, the encapsulated poly (I:C) in anionic liposomes induces a strong immune response without observable hepatic toxicity (FIGS. 9F and G).

Immunization with Anionic Liposomes Containing Poly (I:C) Induces a Stronger T Cell Response Than Free Poly (I:C).

The next aim was to determine if liposomal poly (I:C) induces a stronger T cell response than free poly (I:C). In a previous study (Nijen Twilhaar, Pharmaceutics, 2020, vol. 12, no 12, p. 1138), the present inventors observed that incorporating GM3 ganglioside into antigen-containing anionic liposomes enhances T cell responses after IV immunization. These liposomes contained a long synthetic peptide derived from the model antigen ovalbumin (OVA₂₄₇₋₂₇₉), including CD4- and CD8-T cell epitope. Here, the inventors co-administered to mice the former antigen-containing GM3 liposomes with PBS (control), or free poly (I:C) (10 or 100 μg) or poly (I:C)-containing anionic liposomes (10 μg) with or without GM3 by IV injection. After 7 days, the inventors analyzed the T cell responses in the spleen compartment. Even using a ten times higher dose for free poly (I:C), the co-administration of encapsulated poly (I:C) in anionic liposomes but not free poly (I:C) induced a significantly more robust CD8+-T cell response (FIG. 10A, B) and CD4+-T cell response (FIG. 10C) than only antigen-containing liposomes. The incorporation of GM3 does not significantly affect the activity of the anionic liposomes containing poly (I:C). Thus, these results show that incorporating poly (I:C) in anionic liposomes augments the vaccine effectiveness to yield T-cell responses against model ovalbumin peptide.

After re-stimulation with OVA₂₅₇₋₂₆₄ peptide, the amount of total CD8⁺ T-cells producing interferon-gamma was considerably higher than specific CD8+ T-cells in case of treatment with anionic liposomes containing poly (I:C) (FIGS. 10A and 10B). Effector CD8⁺ T-cells, which express TLR3 receptor, have been shown to produce interferon-gamma upon stimulation with poly (I:C) in an antigen-independent way. To confirm if the administration of poly (I:C) containing anionic liposomes can stimulate CD8⁺ T-cells in an antigen-independent way, the inventors repeated the immunization with 10 μg of poly (I:C) encapsulated in anionic liposomes alone or in combination with a low or a high dose of liposomal antigen (FIG. 11).

The administration of anionic liposomes containing poly (I:C) was sufficient for activating CD4+- and CD8+ T cells. The amount of CD8+ T cells producing interferon-y was significantly high and independent of the administration of the antigen (FIGS. 11A and B). These results are relevant for therapies that rely on CD8+ T cell functionality: The administration of free poly (I:C) has been reported to improve CAR-T therapy activity, immune-checkpoint inhibitors antibodies treatment, chemotherapy, and radiotherapy in mice cancer models. Combining anionic liposomes containing poly (I:C) may further improve the efficacy of such fellow therapies.

### Intravenous Administration of Anionic Liposomes Containing Poly (I:C) has a Strong Anti-Tumor Effect and Overcomes the Resistance to Immune Checkpoint Therapy in Multiple Syngeneic Tumor Models.

Immune checkpoint inhibitors (ICI) are a novel class of immunotherapy drugs that have transformed the treatment of a broad spectrum of advanced untreatable cancers such as metastatic melanoma, triple-negative breast cancer, colorectal cancer and non-small and small lung cancer among others. They block immune checkpoint proteins that stop the immune system from attacking the cancer cells, inducing a long-term durable antitumor response in certain patients. However, the clinical reality is that most patients do not benefit from this revolutionary therapy approach. The response rate is low among different cancer types. ICIs require the presence of sufficient anti-tumoral immune cells in the tumor microenvironment to be effective. ICIs are hardly effective in patients that have a low number of anti-tumoral immune cells in the tumor (‘cold’ tumor). Additionally, the accumulation of immunosuppresive cells (known as myeloid-derived suppressor cells (MDSCs)) in the tumor microenvironment contributes to cancer progression and immunotherapy resistance. Nucleic acid adjuvants are immunostimulants that can increase the infiltration of tumor infiltrating lymphocytes (TILs), including CD8+ and CD4+ T cells and B cells, turning ‘cold’ tumors into ‘hot’ tumors. Currently, there is clinical cancer research on the intratumoral administration of nucleic acids adjuvants encapsulated in cationic nanoparticles in combination with ICIs. However, using this local administration route has a main limitation: most tumors tissues are not accessible for intratumoral injections, and the adjuvant effect only remains local. Intravenous administration is avoided because these cationic delivery systems can be physically unstable and aggregate in the bloodstream (risk for lung embolisation) and cationic molecules can induce strong inflammatory responses.

The present inventors tested the antitumor efficacy of the combination of intravenous anionic liposomes containing poly (I:C) and an immune checkpoint inhibitor, an anti-PD-L1 antibody, in an aggressive metastatic melanoma tumor model (B16F 10) in mice (FIG. 14A). B16F10 melanoma, which is syngeneic for C57BL/6 mice, is a poorly immunogenic tumor with an immunosuppressive microenvironment. It displays a low expression of MHC class I molecules, resulting in low T CD8⁺ infiltration. This tumor model is highly aggressive and one of the most resistant to ICI. The mice treated with the anti-PD-L1 antibody as monotherapy could not delay the tumor growth nor improve the overall survival (FIG. 14A). Remarkably, the combination of the anti-PD-L1 antibody with poly (I:C) (10 μg) encapsulated in anionic liposomes yielded already in 5 days after the first intravenous dosing a strong and significant inhibition of tumor growth (65%) in contrast to the anti-PD-L1 monotherapy group (−17%) and a 100% increase in mean overall survival without apparent toxicity (FIGS. 14 B, C, D and E). Importantly, the therapeutic effects of liposomal poly (I:C) were independent of the initial tumor size (7-238 mm³) (FIG. 5). In the case of the free poly (I:C) (i.e. not encapsulated in liposomes), even a 5 times higher dose (50 μg) did not significantly inhibit tumor growth with no improvement of overall survival when given in combination with the anti-PD-L1.

Based on the improvement of the therapeutic effects observed when poly (I:C) was encapsulated in anionic liposomes in comparison to free poly (I:C), the inventors studied if liposomal poly (I:C) have anti-tumor effects as a monotherapy in two additional models: MC38 coloncarcinoma and Hepa 1-6 Hepatocellular carcinoma. The MC38 coloncarcinoma model is highly aggressive and widely used for testing immunotherapeutics. The mice treated with the anti-PD-L1 antibody as monotherapy could not delay the tumor growth nor improve the overall survival (FIGS. 21A and 21B) while the mice treated with liposomal poly (I:C) monotherapy yielded a strong anti-tumor effect and a 100% increase in the mean overall survival. Impressively, the combination of liposomal poly (I:C) and a-PD-L1 yielded an even stronger and complete response (at least 50% tumor size reduction) in 63% of the mice and long-term cure (60 days) in 38% of the mice. After 60 days of the start of treatment, the cured mice and control naïve mice were re-challenged with fresh MC38 cancer cells. While the new MC38 cancer cells grown aggressively in the naïve mice, the cured mice from the combination group (liposomal poly (I:C) and a-PD-L1) were able to block the growth of the tumor without needing additional treatment (FIG. 21C). This finding points to the development of an immunological memory, which is required for preventing relapses in cancer patients. Additionally, the inventors studied the effects of liposomal poly (I:C) on the immune cell composition of the tumor microenvironment of established colon carcinoma tumors. Flow cytometry analysis of the tumor after 5 days of the start of treatment revealed that the treatment with liposomal poly (I:C) drastically changed the immune cell (CD45+) composition in the tumor microenvironment, with a robust increase of anti-tumor TILs (from 12% to 50%) and a strong decrease of pro-tumor MDSCs (from 30% to 10%) (FIG. 21D). These immune cell composition changes likely explain the strong anti-tumor activity of liposomal poly (I:C) and the synergic effects of combination with ICIs.

Moreover, the inventors studied the effects of liposomal poly (I:C) in an orthotopic hepatocellular carcinoma Hepa 1-6 model. In mice with multiple established tumors, it was observed that liposomal poly (I:C) monotherapy was able to reduce the size and number of tumor nodules, reflecting a strong anti-tumor activity (FIG. 22).

Finally, the inventors studied the advantage of the use of nanoparticles that do not contain cationic molecules for the delivery of nucleic acid adjuvants. In the MC38 coloncarcinoma model, the inventors compared poly (I:C) encapsulated in anionic liposomes with poly (I:C) encapsulated in LNP, the latter being the only nanoparticle formulation approved by FDA for the intravenous delivery of nucleic acids. Poly (I:C) was successfully encapsulated in anionic liposomes (pIC-AL) and LNP (pIC-LNP) employing the same manufacturing process and conditions. The particle size distribution and the RNA encapsulation efficiency were similar for both types of nanoparticles (Table N 1).

TABLE N 1
Characteristics of pIC-AL and pIC-LNP

| | Particle | | | | Zeta potential | | Encapsulation | |
|---|---|---|---|---|---|---|---|---|
| | Size (nm) | | Pdl | | (mV) | | efficiency (%) | |
| | Mean | SD | Mean | SD | Mean | SD | Mean | SD |
| pIC- | 128 | 1 | 0.17 | 0.01 | −42 | 3 | 79 | 4 |
| AL | | | | | | | | |
| pIC- | 130 | 1 | 0.22 | 0.01 | −17 | 3 | 88 | 9 |
| LNP | | | | | | | | |

The inventors also studied the effects of the two formulations in combination with an a-PD-L1 antibody. The administration of 10 μg of poly (I:C) in LNP (piC-LNP) induces severe acute toxicity, which was strongly potentiated when combined with the a-PD-L1 antibody: The animals treated with pIC-LNP showed the following severe adverse events: 1) 16% lethality (monotherapy) and 66% (combination therapy) after 72 hours of start of treatment (FIG. 23 A), 2) all animals had a reduction of at least 15% of body weight (FIG. 23 B) and 3) all animals presented an elevation of the AST hepatic enzyme activity in the bloodstream, suggestive of liver damage (FIG. 24F). No severe adverse events were encountered when using liposomal poly (I:C), while anti-tumor efficacy was similarly strong as in the poly (I:C) LNP groups (FIG. 23C). Due to the severe toxicity observed in the pIC-LNP group, the inventors terminated the experiment, isolated liver, kidneys, heart and spleen and performed histopathological analysis. PIC-LNP treatment induced severe tissue damage in the liver (FIG. 24D) and the spleen, which was potentiated when combined with ICI treatment (FIG. 24E). No tissue damage was observed when poly (I:C) was encapsulated in anionic liposomes as monotherapy (FIG. 24B) and in combination with ICI (FIG. 24C). The analysis of pro-inflammatory cytokine levels in the bloodstream at 3 hours after the injection of poly (I:C)-nanoparticles (FIG. 23D-I) showed a dramatic and significant increase in case of pIC-LNP but not by pIC-AL when compared to the reference levels of PBS group: 1185 fold change increase (pIC-LNP) vs 43-fold change increase (pIC-AL) for IL-6, 1577 fold increase (pIC-LNP) vs 170 fold increase (pIC-AL) for INF-α, 4703 fold change increase (pIC-LNP) vs 107 fold change increase (pIC-AL) for INF-β, 1216 fold change increase (pIC-LNP) vs 16 fold change increase (pIC-AL) for INF-γ, 654 fold change increase for (pIC-LNP) vs 197 fold change increase (pIC-AL) for TNF-α, 7 fold increase (pIC-LNP) vs 6 fold increase (pIC-AL) for IL-1β.

The extreme elevation of pro-inflammatory cytokine plasma levels observed when using pIC-LNP suggests a “cytokine storm” and is likely the underlying cause of lethality and liver damage.

Intravenous Administration of Anionic Liposomes Containing mRNA do not Induce an Immune Inflammatory Response while LNP Containing the Same mRNA do

Previous experiments illustrate that the current state-of-art nanoparticles for nucleic acid delivery (LNP) worsen the immunotoxicity of the nucleic acid adjuvant poly I:C, while the encapsulation in anionic liposomes enhance its efficacy without increasing its immunotoxicity. To validate the non-inflammatory properties of the novel nucleic acid lipid nanoparticles, the inventors studied whether the administration of non immunostimulant mRNA encapsulated in anionic liposomes [EPC: EPG: Cholesterol (3:1:2)] lacks the induction of the immune responses. This is particularly of interest in medical applications where an inflammatory immune response must be avoided, such as for the treatment of an autoimmune disease, diseases where chronic inflammation is involved or a genetic disease. Non immunogenic mRNA was successfully encapsulated in anionic liposomes and LNP employing the same manufacturing process and conditions. The particle size distribution and the RNA encapsulation efficiency were similar for both types of nanoparticles (Table N 2).

TABLE N 2
Characteristics of mRNA-AL and mRNA-LNP

| | Particle | | | | Zeta potential | | Encapsulation | |
|---|---|---|---|---|---|---|---|---|
| | Size (nm) | | Pdl | | (mV) | | efficiency (%) | |
| | Mean | SD | Mean | SD | Mean | SD | Mean | SD |
| mRNA- | 120 | 3 | 0.20 | 0.01 | −31 | 2 | 80 | 9 |
| AL | | | | | | | | |
| mRNA- | 137 | 4 | 0.14 | 0.01 | −14 | 1 | 99 | 7 |
| LNP | | | | | | | | |

A single dose of 10 μg of mRNA encapsulated in anionic liposomes or LNP were administered intravenously in healthy mice. After 24 hours, the immune response in the spleen compartment was analyzed. The administration of mRNA-LNP induced the activation of splenic dendritic cells type 1 and 2 (increase of the expression of the CD80 marker) and the activation of splenic T cells (increase of the expression of the CD25 marker), while the administration of mRNA-AL did not induce the activation of splenic dendritic cells type 1 and 2 and T cells (FIG. 25A-C). Additionally, the analysis of pro-inflammatory cytokine levels in the bloodstream at 3 hours after the injection of mRNA nanoparticles (FIG. 23 D-I) showed a dramatic and significant increase in case of mRNA-LNP but not by mRNA-AL when compared to the reference levels of PBS group: 669 fold increase (mRNA-LNP) vs 4 fold change increase (mRNA-AL) for IL-6, 579 fold change increase (mRNA-LNP) vs no increase (mRNA-AL) for INF-α, 60 fold change increase (mRNA-LNP) vs no increase (mRNA-AL) for INF-β, 15 fold change increase (mRNA-LNP) vs no increase (mRNA-AL) for INF-γ, 8 fold change increase (mRNA-LNP) vs 2 fold change increase (mRNA-AL) for TNF-α, 2 fold change increase vs no increase (mRNA-AL) for IL-1β. These findings validate the non inflammatory nature of anionic liposomes containing nucleic acids.

### CONCLUSION

In conclusion, it is possible to encapsulate multiple nucleic acids such as dsRNA (poly (I:C)) and ssRNA (mRNA) in anionic liposomes using solvent mixture techniques or microfluidics technology. The extensive physicochemical characterization studies indicate that the RNA is to a certain extent localized within the hydrophobic region of the lipid bilayers. This may explain the enhanced stability of RNA in anionic liposomes. The encapsulation of poly (I:C) in anionic liposomes potentiates its immunostimulatory effect without noticeable toxicity when compared to the current state-of-art delivery system, RNA-LNP. The encapsulation of poly (I:C) in anionic liposomes renders in a strong anti-neoplastic agent by increasing the localization of tumor infiltrating lymphocytes into the tumor tissue, making it well-suited for the treatment of malignancies alone as monotherapy and in a combination therapy with ICIs. Additionally, the invention facilitates the use as an adjuvant system in combination with an antigen to improve the effectiveness of vaccines. Moreover, the encapsulation of a non-immunogenic mRNA in anionic liposomes does not trigger the activation of the immune system, while mRNA in LNP does. This unique feature enables the use of anionic liposomes containing RNA for the treatment of diseases with an underlying inflammatory signature.

1. Composition comprising anionic nucleic acid-lipid particles, wherein the anionic nucleic acid-lipid particles comprise:
one or more nucleic acid;
one or more lipid bilayer comprising at least one anionic lipid and/or at least one neutral lipid,
wherein at least part of the one or more nucleic acid is encapsulated by the nucleic acid-lipid particles and less than 10 wt. % of the one or more nucleic acid is exposed on the outside of the nucleic acid-lipid particles.

2. Composition according to claim 1, wherein
at least 20 wt. % of the one or more nucleic acid is encapsulated by the nucleic acid-lipid particles;
at least 20 wt. % of the one or more nucleic acid is comprised in the hydrophobic region of the one or more lipid bilayer, preferably in the form of nucleic acid-lipid complexes; and/or
less than 5 wt. % of the nucleic acid is exposed on the outside of the nucleic acid-lipid particles.

3. Composition according to any one of the previous claims, wherein the particles comprise at most 20 mol. % of one or more of cationic lipid, cationic polymer, and multivalent cation, relative to total mol lipid of the particles.

4. Composition according to any one of the previous claims, wherein the particles comprise at least 5 mol. % of at least one sterol, relative to total mol lipid of the particles.

5. Composition according to any one of the previous claims, wherein the particles comprise
at least 0.5 mol. %, preferably at least 5 mol. %, more preferably at least 15 mol. % of at least one anionic lipid, relative to total mol lipid of the particles; and/or
at least 1 mol. %, preferably at least 5 mol. %, more preferably at least 15 mol. % of at least one neutral lipid, relative to total mol lipid of the particles.

6. Composition according to any one of the previous claims, wherein the nucleic acid-lipid particles
have core shell structure and/or a core encapsulated by the one or more lipid bilayer, wherein the core comprises at least 50 vol. % aqueous solvent, relative to total core volume;
do not contain cationic lipid and/or comprise at most 1 mol. % cationic lipid relative to total mol lipid of the particles;
do not contain cationic polymer and/or comprise at most 1 mol. % cationic polymer relative to total mol lipid of the particles; and/or
do not contain multivalent cation and/or comprise at most 1 mol. % multivalent cation relative to total mol lipid of the particles.

7. Composition according to any one of the previous claims, wherein the nucleic acid-lipid particles
have an average hydrodynamic diameter of 50-300 nm as determined by dynamic light scattering (DLS);
have a polydispersity index (PDI) of 0.01-0.5 as determined by dynamic light scattering (DLS); and/or
have a zeta potential of between −100 and −10 mV as determined by electrophoretic light scattering (ELS).

8. Composition according to any one of the previous claims, wherein the one or more nucleic acid is a DNA, RNA or an DNA or RNA analogue, and/or wherein the one or more nucleic acid is an immunologic adjuvant and/or a toll-like-receptor agonist (TLR), preferably a TLR3, TLR7/8 or TLR9 agonist, more preferably a TLR3 agonist, most preferably poly (I:C), poly (C:G) or poly (A:U),
and wherein the one or more lipids are a neutral lipid, an anionic lipid, and a sterol, more preferably one or more lipids are a phosphatidylcholine, phosphatidylglycerol and a sterol, most preferably EPC, EPG and cholesterol.

9. Composition according to any one of the previous claims, wherein the particles comprise 0.5-40 mol. % of at least one anionic lipid, relative to total mol lipid of the particles and/or wherein the composition is an immunostimulatory composition.

10. Composition according to any one of the previous claims, wherein the particles comprise 0.5-80 mol. % of at least one anionic lipid, relative to total mol lipid of the particles and/or wherein the composition is an immunosilent composition and/or wherein the particles are non-immunogenic in that they do not result in activation of dendritic cells, activation of T cells and/or secretion of IFN-alpha.

11. Composition according to any one of the previous claims, wherein the composition is in combination with one or more selected from the group consisting of an antigen, an immunologic adjuvant and/or a vaccine.

12. Composition according to any one of the previous claims, wherein the composition is in combination with an anti-neoplastic agent.

13. Composition according to claim 12, wherein the anti-neoplastic agent is
a cell-based anti-neoplastic agent;
a lymphocyte-based anti-neoplastic agent, preferably chosen from B cells, αβT cells, γδT cells, NK cells, NKT cells, autologous tumor-infiltrating lymphocytes (TILs), autologous NK cells, CAR-T cells, CAR-B cells, CAR-NK cells, CAR-NKT cells;
myeloid-based anti-neoplastic agent, preferably chosen from dendritic cell-based anti-neoplastic agent, macrophage-based anti-neoplastic agent or a neutrophil based anti-neoplastic agent,
an antibody, or an immune checkpoint inhibitor, wherein the immune checkpoint inhibitor is preferably an antibody, more preferably α-CTLA4 antibody, anti-PD1 antibody and/or anti-PD-L1 antibody;
a small molecule drug preferably chosen from alkylating agent, antibiotic, anti-metabolite, hormonal antagonist, photosensitizer, protein kinase inhibitor, poly (ADP-ribose) polymerase inhibitor, taxane and/or topoisomerase inhibitor,
radiation therapy;
a cytokine preferably chosen from IL-2,,IL-12, IL-15 and IL-21;
growth factor preferably chosen from CSF family, and FIt3L; and/or
a steroidal or a non-steroidal anti-inflammatory drug.

14. Composition according to any one of the previous claims, for use in preventing and/or treating a disease.

15. Composition for use according to claim 14, wherein the disease is one or more of cancer, infection, chronic inflammation, genetic disease and autoimmune disease.

16. Method for preparing a composition comprising nucleic acid-lipid particles according to any one of claims 1-15, the method comprising
a) providing one or more organic solution comprising at least one anionic lipid and/or at least one neutral lipid;
b) providing one or more aqueous solution comprising one or more nucleic acid;
c) combining the one or more organic solution and the one or more aqueous solution, thereby producing the composition comprising nucleic acid-lipid particles according to any one of claims 1-15.

17. Method according to claim 16, wherein the combining in step c) involves laminar flow mixing of the one or more organic solution and the one or more aqueous solution.

18. Method according to any one of claims 16-17, wherein:
the flow rate ratio between the one or more aqueous solutions and the one or more organic solutions is between 24:1 and 1:10;
the at least one anionic lipid and/or the at least one neutral lipid is provided in the organic solution in a concentration of 0.1-50 mM;
the one or more organic solution is an ethanol solution comprising at least 80% (v/v), preferably at least 90% (v/v), more preferably at least 97.5% (v/v), most preferably at least 99% (v/v) ethanol; and/or
the one or more nucleic acid is provided in the aqueous solution in a concentration of 10-5000 μg/ml.

19. Method according to any one of claims 16-18, wherein the one or more organic solution in step a) comprises:
at least 20 mol. % of at least one anionic and/or at least one neutral lipid, relative to total mol lipid in the one or more organic solution;
at most 10 mol. % of at least one cationic lipid, relative to total mol lipid in the organic solution; and/or
at least 5 mol. % of at least one sterol, relative to the total mol lipid in the organic solution.

20. Method according to any one of claims 16-19, wherein:
the organic solution in step a) is free of cationic lipids and/or comprises at most 1 mol. % of at least one cationic lipid relative to total mol lipid in the organic solution; and/or
the method does not involve the use of a cationic lipid and/or a cationic polymer.
